SEQUENCE LISTING (99 sequences)

SEQ ID NO: 1 - DNA, 963 bp, Artificial Sequence
  Rhizopus oryzae sequence codon optimized for Saccharomyces cerevisiae
atggtcctgcatagtaaggtggcaatagtcggagccggtgccgtaggcgcttcaacagcctacgcgctaatgtttaagaatatatgcacagaaattatagtggtcgatgttaatcctgatattgtgcaagcgcaggtactagacttagccgacgctgcttcaatatctcacactccaatcagggcaggatctgttgaagaagctggtcaagcagacatcgtggtaataaccgcgggtgcaaagcaaagagaaggcgaaccgagaacaaaacttattgaaaggaactacagggttctacaatcaattatcggtggtatgcaacctatccgtcccgacgctgtgattttagttgttgcaaacccagttgacatactaacacacattgctaaaactctatcaggcttgcctcccaatcaagtgattggctcaggaacgtacctggatacaacaaggttaagggtacatttgggcgatgtattcgacgtgaatccccagagcatccacgcctttgtgctgggtgagcacggcgactcacagatgatagcctgggaggccgcgtctatcggagggcagccccttactagctttcccgagttcgcgaagttagacaaaaccgcaattagcaaggccatttcaggaaaggcaatggagataattcgtctgaagggagcgacattttatgggatcggcgcttgtgccgcggacttggttcacactattatgttgaataggaagtccgttcacccggtctcagtttacgtggaaaaatatggcgcgacattttctatgccggccaaacttggatggagaggagttgaacaaatttacgaggtacctcttactgaagaagaagaagcgctacttgtgaagtctgtcgaggcgctgaagtctgttgagtatagcagtacgaaggttcctgagaaaaaggtccatgcaacatcatttagtaaaagtaactgctga

SEQ ID NO: 2 - protein, 300 aa, Rhizopus oryzae
MVLHSKVAIVGAGAVGASTAYALMFKNICTEIIVVDVNPDIVQAQVLDLADAASISHTPIRAGSVEEAGQADIVVITAGAKQREGEPRTKLIERNYRVLQSIIGGMQPIRPDAVILVVANPVDILTHIAKTLSGLPPNQVIGSGTYLDTTRLRVHLGDVFDVNPQSIHAFVLGEHGDSQMIAWEAASIGGQPLTSFPEFAKLDKTAISKAISGKAMEIIRLKGATFYGIGACAADLVHTIMLNRKSVHPVSVYVEKYGATFSMPAKLGWRGVEQIYEVPLTEEEEALLVKSVEALKSVEY

SEQ ID NO: 3 - protein, 320 aa, Lachancea fermentati
MEENQEATVAGPVKIVVIGVGSVGSATAYTLLLSGIVNEIVLIDINKDKAEGESMDLNHAAPSITKSRAGDYADCAGAAIVIVTCGINQKHGQTRMDLAAKNAKIMQDIIPNVAKYAPDTILLIATNPVDVLTYISYKASGFPLNRVIGSGTVLDTARFKYILGDHFKISSDSIDACVIGEHGDSGVPVWSLTNIDGMKLRDYCEKSKQDFDEEAFQKIFEQTRDAAYDIIKRKGYTSYGIAAGLLRIVKAILEDTDSVLTVSTVGDYFGVEQIAISVPTRLNKSGAHQAAELTLDENEIELMKKSAGEIKSVLKNLNIA

SEQ ID NO: 4 - protein, 326 aa, Lachancea fermentati
MSKYSQKSILKTDLRPATIAIVGVGSVGATTAYTLQLSGMIADIVLIDINKDKAEGEYMDLNHAAPMTTETQVRVGEYSDCVDAAIVIIAGGANQKPGQTRMELAAKNAKIIEEIIPNVVKYAPDTILLIATNPVDVLTYASYKLSGFPTSRVIGSGTLLDSTRLRCNIGRYYNISSDSVDACIIGEHGDSELAVWSRATIAGMNLAEFSAATKQEYDKESLHKIFEHTRDAAYNIIKRKGYTAYGIAAALTRIVEAVLRDEGSLLTVSIVGDYYGIKNVALSVPTKVGRDGAHHIMDLTLNEKENEEVKNSAKKIRAACDSLGLK

SEQ ID NO: 5 - protein, 326 aa, Lachancea fermentati
MSKSTQSPALKTDLRPATIAIVGVGSVGATTAYTLQLSGMVADIVLIDINKDKAEGEFMDLNHAAPMTTETQVRVGDYPDCADAAIVIITGGANQKPGQTRMELAAKNAKIMEEIIPNVVKYAPDTILLIATNPVDVLTYASYKLSGFPASRIIGSGTLLDSTRLRCNIGRYYGISSDSVEASIIGEHGDSELAVWSRATIAGMSLAEFSAATQQKYNKESLHKIFEHTRDAAYNIIKRKGYTAYGIAAALTRIVEAILRDEGSLLTVSIVGNYYGVKDVALSVPTKVGRDGAHHIMDLILNQDETDRMKSSAKKIRSACESLGLD

SEQ ID NO: 6 - protein, 324 aa, Lachancea fermentati
MYQDKSMRATVEHAKLIKIVIVGVGSVGSATAYTLILSGIVNDLVLIDVNREKAEGESMDLNHAAPSWASARAGNYSDCKDADIVIITCGVSQTNGQTRMDLAAKNTDIMRDVVSNVAQNAPDTILLIATNPVDVLTYVSYEASGFPLHRVIGSGTVLDTARFKHILGNHFKVPSNTVEACVIGEHGDSSVAAWSLTNIKGMKLREYCEKSNQKFDEEAFQKVFEQTRDAAYDIIKRKGCTSYGIAAGLLRIVRAILANEDHVLPVSTVGAHFGIDHVALSVPTKLNRGGAYPAGALTINEREMELARKSAEKIKSVLDKVFIK

SEQ ID NO: 7 - protein, 326 aa, Lachancea thermotolerans
MAQNKESSTMKAGLNPVKVVVVGTGSVGSTTAYTLLLSGMVAEIVLIDINKDKADGEGMDLNHAAPMTTDCRVYVGDYPDCANAAIVIITGGANQKPGQTRMDLAAKNAKIMQEIIPNIVKNAPDTILLLATNPVDVLTSISYKLSGFPASRVIGSGTLLDSARLRYNLSKYYNISSESIGAFIIGEHGDSELPVWSLASIAGMRLRDYCEKSNQKFDQDALEKIFEKTRSAAYDIIKRKGYTAYGIAAGLVRIVETILKNEGSLLTVSTVGDYFGVKQVALSVPTKVDRTGAHHIVGLSLDDNEIEEVKKSGQNIKSVCSRLGFD

SEQ ID NO: 8 - protein, 327 aa, Lachancea thermotolerans
MPHSIYPPASTIKKVNPVKVAVIGVGSVGSTTAYTLLFSEMISEVVLIDINTHKAEGESMDLNHAAPSTTGSVVYVGDYSDCADAAIVIITGGANQKPGQTRMDLAATNARILQGIIPKIVEYAPKTILLIATNPVDVLTYVSYKVSGFPLNRVIGSGTLLDSARLKYHLSQHFKTSSKNVDAFIVGEHGDSSLPVWSHVKISGIRLRDYCEQSQQAYDHELFHQMFEKTRNAAYDIIERKGYTAYGIAAGILRIVETILKDGGSPLTVSTVGNYFGIEQVALSVPTKLNRNGAHSPVDLSFDIKEIELIKKSALQIKSVCSTLELL

SEQ ID NO: 9 - protein, 324 aa, Lachancea thermotolerans
MFQDTKSQAVRTDAKTVKVVVVGVGSVGSATAYTLLLSGIVSEIVLIDVNKDKAEGESMDLNHAAPSNTRSRAGEYPDCAGAAIVIVTCGINQKNGQTRMDLAAKNANIMLEIIPNVAKYAPDTILLIATNPVDVLTYISYKASGFPLSRVIGSGTVLDTARFKYILGEHFKISSDSIEACVIGEHGDSGVPVWSLTNIDGMKLRDYCEKANHIFDQNAFHRIFEQTRDAAYDIIKRKGYTSYGIAVGLLRIVKAILEDTGSTLTVSTVGDYFGVEQIAISVPTKLNRSGAHQVAELSLDEKEIELMEKSASQIKSVIEHLEIN

SEQ ID NO: 10 - protein, 225 aa, Lachancea fermentati
MELAAKNAKIMEEIIPNVVKYAPDTILLIATNPVDVLTYASYKLSGFLASRIIGSGTLLDSTRLRCNIGRYYGISSDSVEASIIGEHGDSELAVWSRATIAGMSLAEFSAATQQKYDKESLHKIFEHTRDAAYNIIKRKGYTAYGIAAALTRIVEAILRDEGSLLTVSIVGNYYGVKDVALSVPTKVGRDGAHHIMDLILNQDETDRMKSSAKKIRSACESLGLD

SEQ ID NO: 11 - protein, 333 aa, Wickerhamomyces anomalus
MKVTVCGAAGGIGQPLSLLLKLSPYVTELSLYDVVNSPGVGADLSHIDTNTKIQSFLPDNDGLSKALQNTDIVIIPAGVPRKPGMTRDDLFAINAGIVRDLAQGVASFAPDAFVLVISNPVNSTVPIFAEVLKKNGVFNARKLFGVTTLDLVRANTFISQLSNDSLNPENLKIPVIGGHSGDTIVPLFSLGSPEFYKSLSIEQRDALIHRVQFGGDEVVQAKKGMGSATLSMAYSGFKLAEELMKAIKGDSSIVDSSFVYLSDDIKGAKEVRQLLPDLDYISLPVKLSAKGVESIEIDVLNKINDHEKELLNVAIDQLKTNISKGINFIKNQA

SEQ ID NO: 12 - protein, 343 aa, Arabidopsis thaliana
METDLKSTFLNVYSVLKSDLLHDPSFEFTNESRLWVDRMLDYNVRGGKLNRGLSVVDSFKLLKQGNDLTEQEVFLSCALGWCIEWLQAYFLVLDDIMDNSVTRRGQPCWFRVPQVGMVAINDGILLRNHIHRILKKHFRDKPYYVDLVDLFNEVELQTACGQMIDLITTFEGEKDLSKYSLSIHRRIVQHKTAYYSFYLPVACALLMAGENLENHIDVKNVLVDMGIYFQVQDDYLDCFADPETLGKIGTDIEDFKCSWLVVKALERCSEEQTKILYENYGKTDPSNVAKVKDLYKELDLEGVFMEYESKSYEKLTGAIEGHQSKAIQAVLKSFLAKIYKRQK

SEQ ID NO: 13 - protein, 342 aa, Glycyrrhiza uralensis
MADLKSTFLNVYSVLKSELLHDPAFEWSDDSRQWADRMLDYNVPGGKLNRGLSVIDSYRLLKEGQALNDDELFLASALGWCIEWLQAYFLVLDDIMDNSHTRRGQPCWFRVPKVGMIAANDGVLLRNHIPRILRKHFRGKPYYVDLLDLFNEVEFQTASGQMIDLITTLEGEKDLSKYTLSLHRRIVQYKTAYYSFYLPVACALLMMGENLDNHIDVKNILVDMGTYFQVQDDYLDCFGDPQTIGKIGTDIEDFKCSWLVVKALELCNEEQKKVLYEHYGKPDPTNVAKVKALYNELNLQGVFAEYESASYEKLVTSIEAHPSKAVQSVLKSFLAKIYKRQK

SEQ ID NO: 14 - protein, 342 aa, Capsella rubella
MADLKSTFLDVYSILKSELLQDPSFEFTHESRQWVERMLDYNVRGGKLNRGLSVVDSYKLLKQGQDLTDKETFLACALGWCIEWLQAHFLVLDDIMDNSVTRRGQPCWFRQPKVGMIAINDGVLLRNHIHRILRKHFREMPYYVDLVDLFNEVEFQTASGQMIDLITTFDGEKDLSKYSLQIHRRIVQYKTSYYSFYLPVACALFMAGENLETHTDVKNVLVDMGIYFQVQDDYLDCYADPETLGKIGTDIEDFKCSWLVVKALERCSEEQTKILYENYGKSEPSNIAKVKALYKELDLEAAFMEYEKESYEKLTKLIEAHQSKAIQAVLKSFLAKIYKRQK

SEQ ID NO: 15 - protein, 342 aa, Arabidopsis thaliana
MADLKSTFLNVYSVLKAELLQDPAFEYTDVSRQWIERMLDYNVPGGKLNRGLSVIDSYRLLKEGKELTEDEIFLASVLGWCIEWLQAYFLVLDDIMDNSVTRRGQPCWFRVPKVGMIAVNDGVLLRNHIPRMLKNHFKGKPYYVNLLDLFNEVEFQTASGQMIDLITTIEGEKDLSKYALSLHRRIVQYKTAYYSFYLPVACALVMSGEDLENHTVVKDTLVQMGIYFQVQDDYLDCFGDPETIGKVGTDIEDFKCSWLVVKALELCNEEQKKLLHEAYGKPDPENVAKVKALYKELNIEGVFAEYESQSYAELNATIEAHPSKSIQAVLKSFLGKIYKRQK

SEQ ID NO: 16 - protein, 342 aa, Lupinus angustifolius
MADLKSSFLKVYSILKSELLHDPAFEFSNDSRQWVERMVDYNVPGGKLNRGMSVIESYRLLKDGQELHDDEIFLASALGWCIEWLQAHFLVSDDIMDNSHTRRGQPCWFRVPKVGLIAANDGLLLRNHIHRIFKNHFRGKPYYVDLLHLFNEVEFQTVSGQMIDLITTLEGEKDLSKYTLSLHRSIVQYKTTYYSFYLPVACALLMLGENLDNHINVKNILVEMGTFFQVQDDYLDCFGAPETIGKIGTDIEDFKCSWLVVKALELSNEEQKKVLNENYGKPDLANVSKVKALYNELNLQGVFAEYESNSYEKLVASIEAHPSKAVQAALKSFLAKIYKRQK

SEQ ID NO: 17 - protein, 548 aa, Citrus sinensis
MSSGETFRPTADFHPSLWRNHFLKGASDFKTVDHTATQERHEALKEEVRRMITDAEDKPVQKLRLIDEVQRLGVAYHFEKEIGDAIQKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLCNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 18 - protein, 555 aa, Citrus junos
MSAQVLATVSSSTEKTVRPIAGFHPNLWGDYFLTLASDCKTDDTTHQEEYEALKQEVRSMITATADTPAQKLQLVDAVQRLGVAYHFEQEIEDAMEKIYHDDFDNNDDVDLYTVSLRFRLLRQQGFKVPCDVFAKFKDDEGKFKASLVRDVHGILSLYEAGHLAIRGEGILDEAIAFTRTHLQSMVSQDVCPNNLAEQINHTLDCPLRRALPRVETRFFLSVYPRDDKHDKTLLKFSKLDFNLVQRIHQKELSAITRWWKDLDFTTKLPYARDRIVELYFWIVGTYFEPKYTLARKIMTKTIYTASIIDDTFDAYGFFEELKLFAEAVQRWDIGAMDILPEYMKVLYKALLDTFNEIEQDLAKEGRSSCLPYGKEKMQELVQMYFVQAKWFSEGYVPTWDEYYPVGLVSCGYFMLATNSFLGMCDVANKESFEWISRTLRFQQRHQFICRLRNDIVSHQFEQKRGHIASGVECYIKQYGVSAEEVVTVFTEEVENAWKDMNEEFLKPTAFPVALIERPFNIARVIEFLNKKGDWYTHSHAIKDQIAAVLRDPVTI

SEQ ID NO: 19 - protein, 555 aa, Vitis vinifera
MSIQVSTCPLVQIPKPEHRPMAEFHPSIWGDQFIAYTPEDEDTRACKEKQVEDLKAEVRRELMAAAGNPSQLLNFIDAVQRLGVAYHFEREIEESLQHIYDRFHDADDTEDDLYNIALQFRLLRQQGYNISCGIFNKFKDEKGSFKEDLISNIQGMLGLYEAAHLRVHGEDILEEALAFTTTHLKATVESLGYPLAEQVAHALKHPIRKGLERLEARWYISLYQDEASHDKTLLKLAKLDFNLVQSLHKEELSNLARWWKELDFATKLPFARDRFVEGYFWTLGVYFEPQYSRARRILTKLFSMASIIDDIYDAYGTLEELQPFTEAIERWDIKSIDHLPEYMKLFYVTLLDLYKEIDQELEKYGNQYRVYYAKEVLKSQVRAYFAEAKWSHEGYIPTIEEYMLVALVTSGSCILATWSFIGMGEIMTKEAFDWVISDPKIITASTVIFRLMDDITTHKFEQKRGHVASGIECYMKQYGVSEEQVYSEFHKQVENAWLGINQECLKPTAVPMPLLTRVVNLSRVMDVIYKEGDGYTHVGKVMKDNIGSVLIDPIV

SEQ ID NO: 20 - protein, 589 aa, Callitropsis nootkatensis
MAEMFNGNSSNDGSSCMPVKDALRRTGNHHPNLWTDDFIQSLNSPYSDSSYHKHREILIDEIRDMFSNGEGDEFGVLENIWFVDVVQRLGIDRHFQEEIKTALDYIYKFWNHDSIFGDLNMVALGFRILRLNRYVASSDVFKKFKGEEGQFSGFESSDQDAKLEMMLNLYKASELDFPDEDILKEARAFASMYLKHVIKEYGDIQESKNPLLMEIEYTFKYPWRCRLPRLEAWNFIHIMRQQDCNISLANNLYKIPKIYMKKILELAILDFNILQSQHQHEMKLISTWWKNSSAIQLDFFRHRHIESYFWWASPLFEPEFSTCRINCTKLSTKMFLLDDIYDTYGTVEELKPFTTTLTRWDVSTVDNHPDYMKIAFNFSYEIYKEIASEAERKHGPFVYKYLQSCWKSYIEAYMQEAEWIASNHIPGFDEYLMNGVKSSGMRILMIHALILMDTPLSDEILEQLDIPSSKSQALLSLITRLVDDVKDFEDEQAHGEMASSIECYMKDNHGSTREDALNYLKIRIESCVQELNKELLEPSNMHGSFRNLYLNVGMRVIFFMLNDGDLFTHSNRKEIQDAITKFFVEPIIP

SEQ ID NO: 21 - protein, 553 aa, Populus trichocarpa
MSTQVSQEVVPKAHHNEIIRRTANYHPSIWGDQFISHLPKDKVHEAIELQEIEKLREQFKRELLAAASNSSQQLDLIDAIQRLGVAYHFETEIEEALQHIYNNRIDMEDDDLYNTALGFRLLRQHGYNVSCDIFNKFKDDKGYFKQSNDVRGILGLYEAAHLAVHGEDILDEALAFTTIHLKSMASSPNCPLTAKVSHALKQPIQRGVPRLESRRYISIYQDEPSCNKTLLRLAKLNFNLVQELHKEELAEITRWWKGLDFARRLPFARDRVVECFFWIVGVYFEPQYSLARKILTKVIAMTSIIDDIYDVYGTLEELELFTEAIDRWDTKSMDQLPDYMKICYEALLNVFSEIEEKVAKEGWSYRVHYGKDAMKVLVHAYFNEAKWFHENHIPTMEEYMQVALVTSGYSMLTTVSFIGMGDMVTKQAFDWVFNHPKIIRASETIGRLLDDVKSHKFEQERGHAASGVECYIRQYGLSEQEVYKEFHMQVVNAWKDINEECLKPTAVPTPLLERILNLSRVIDVIYKEEDGYTHVGKVMKNNVASLLINSVPI

SEQ ID NO: 22 - protein, 396 aa, Bacillus subtilis
MNVLNRRQALQRALLNGKNKQDAYHPFPWYESMRKDAPVSFDEENQVWSVFLYDDVKKVIGDKELFSSYMPQQSSAIGNSIINMDPPRHTQIRSVVNKAFTPRVMKQWEPRIQEITDELIQKFQGRSEFDLVHDFSYPLPVIVISELLGVPSEHMDQFKTWSDLLVSTPKDKSEEAEEAFLEERNKCEEELAAFFANIIEEKRNKPAQDVISILVEAEETGEKLSGEELVPFCTLLLVAGNETTTNLISNAMYSILETPDVYNVLRSHPELTPQAVEEALRFRAPAPVLRRIAKRDTEIGGHLIKEGDMVLAFVASANRDETKFDRAHLFDIHRHPNPHIAFGHGIHFCLGAPLARLEAKIALTSLISAFPHMECVSITPIENSVIYGLKSFRVKM

SEQ ID NO: 23 - protein, 406 aa, Bacillus amyloliquefaciens
MSNLRSPRQAIQRTLMKGKDGLDVYNPFPWYEKMRRESPIQFDEETKVWSVFLYDDAKKVISDKETFSSLMSDMKSSIAKSMLNMDPPKHTQIRSAVNRAFTPRVLKEWEPRIEEITDNLLKQAKNKGRIDIVKDLSYPLPVIVISELLGVPSEHMDQFKKWSDILVSMPKDASPEEAEKNQQERDQCETELAAFFAEIIDSKRKQPGQDIISILIKEEEEGEKLSAEDLIPFCNLLLVAGNETTTNLISNAVYSILETPGLYEELRQDPSLIAQTVEETLRFRAPAPFVRRTVRHDTELRGRRLKSGDIVLCYIASANRDENKFEQADVFDIHRQSNPHLSFGFGVHFCLGAPLARLEAEVALRGIVKAFSHLEPVRVEPIQNSVMYGLDSLEAEINENRGEEKR

SEQ ID NO: 24 - protein, 396 aa, Bacillus halotolerans
MNVVNRRQALQRALMNGKNKQDAYHPFPWYESMRKEAPVTYDEDNQVWSVFLYDDVKKVIGDKELFSSYMPEQTSSIGNSIINMDPPRHTQIRSVVNRAFTPRVMKQWEPRIQDIADDLIHRFHGRSEFDLVQDFSYPLPVIVISELLGVPSEHMDAFKTWSDILVSTPRDGSEEAEKAFLKERNKCEEELAAFFAGIIEEKRKKPEQDIISILIKAEETGEKLSGEELIPFCTLLLVAGNETTTNLISNAMYSILDTPGAYDELQRHPELVPQAVEEALRFRAPAPVLRRIAKRDTEIGGHQIKEGDMVLAFVASANRDEAKFDRAHKFDIHRHPNPHIAFGHGIHFCLGAPLARLEAAIALTALTQTFSHMEPVSITPIENSVIYGLKSFRVNI

SEQ ID NO: 25 - protein, 398 aa, Bacillus nakamurai
MSKFKNPRQAVQKTLMKGKNGLDAYDPFPWYEKMRRENPVHFDEENKVWSVFLYDDVKKVIGDKEAFSSHRLQMTSSIGNSILNMDPPKHTRIRSVVNRAFTPRVLKEWEPRIQQITDDLLKQAQGRGSIDIVKDLSYPLPVIVISELLGVPSEHMEQFKEWSDILVSTPKDESPEAAKVNEQEQDQCERELASFFARIIEEKRNQPGQDIISILIKAEEEGEKLSGEDLIPFCTLLLMAGNETTTNLISNAVYSIAETPGLYEELRQDPSLIPQVIEETLRFRAPAPFVRRTVKHDIELSGRLLKSGEMVLGYIASANRDEKKFEHADVFDIRRQPNPHLSFGFGIHFCLGAPLARLEAEVALKGLLKTYHKLKPVRVEPIQNSVMYGLTAFEAETN

SEQ ID NO: 26 - protein, 406 aa, Bacillus velezensis
MSKLRSTRQAIQRTLMKGKNGLDVYNPFPWYEKMRRESPIYFDEETKVWSVFLYDDVKKVISDKETFSSRMTDVKSSIAKSMLNMDPPKHTQIRSAVNRAFTPRVLKEWEPRIKDITAHLLKQAKNKGRIDIVKDLSYPLPVMVISELLGVPSERMDQFKKWSDILVSMPKDASPEAAEKNQQERDQCEAELAAFFAGIIESKRKQPGQDIISILIKEEEEGEKLTPEDLIPFCNLLLVAGNETTTNLISNAVYSILETPGLYEELRQDPSLIAQTVEETLRFRAPAPFVRRTVRHDTELRGRRLKSGEIVLCYVASANRDENKFEKAGVFDIHRQSNPHLSFGFGVHFCLGAPLARLEAEAALKGIVKTFSHLEPVRIEPIRNSVMYGLESLEAEINENEGEEKR

SEQ ID NO: 27 - protein, 502 aa, Hyoscyamus muticus
MQFFSLVSIFLFLSFLFLLRKWKNSNSQSKKLPPGPWKLPLLGSMLHMVGGLPHHVLRDLAKKYGPLMHLQLGEVSAVVVTSPDMAKEVLKTHDIAFASRPKLLAPEIVCYNRSDIAFCPYGDYWRQMRKICVLEVLSAKNVRSFSSIRRDEVLRLVNFVRSSTSEPVNFTERLFLFTSSMTCRSAFGKVFKEQETFIQLIKEVIGLAGGFDVADIFPSLKFLHVLTGMEGKIMKAHHKVDAIVEDVINEHKKNLAMGKTNGALGGEDLIDVLLRLMNDGGLQFPITNDNIKAIIFDMFAAGTETSSSTLVWAMVQMMRNPTILAKAQAEVREAFKGKETFDENDVEELKYLKLVIKETLRLHPPVPLLVPRECREETEINGYTIPVKTKVMVNVWALGRDPKYWDDADNFKPERFEQCSVDFIGNNFEYLPFGGGRRICPGISFGLANVYLPLAQLLYHFDWKLPTGMEPKDLDLTELVGITIARKSDLMLVATPYQPSRE

SEQ ID NO: 28 - protein, 514 aa, Nicotiana attenuate
MQLSFEEYQLTKMQLFSLVSIFLFLSFLFLLRKWKNSNSQSKKLPPGPWKLPILGSMLHMVGGLPHHVLRDLAKKYGPLMHLQLGEVSAVVVTSPDMAKEVLKTHDIAFASRPSLLAPEIVCYNRSDLAFCPYGDYWRQMRKICVLEVLSAKNVRTFSSIRRDEVLRLINFIRSSSGEPVNVTERIFLFTSSMTCRSAFGQVFKDQDKFIQLIKEVILLAGGFDVADIFPSLKFLHVLSGMKGKIMNAHHKVDAIVENVINEHKKNLATGKTNGALGGEDLIDVLLRLMNDGGLQFPITNDNIKAIIFDMFAAGTETSSSTIVWAMVEMVKNPTVFTKAQAEVREAFRGKETFDENDVEELSYLKLVIKETLRLHPPVPLLLPRECREETNINGYTIPVKTKVMVNVWALGRDPKYWDDAEIFKPERFEQCSKDFVGNNFEYLPFGGGRRICPGISFGLANAYLPLAQLLYHFDWKLPAGIKPSDLDLTELVGVTAARKSDLYLVATPYQPPQK

SEQ ID NO: 29 - protein, 500 aa, Solanum tuberosum
MQLVSIFLFISFLFLLRKWKKYLNNSQTKKLPPGPWKLPFIGSMHHLAGGLPHRVLRDLAEKYGPLMHLQLGEVSAVVVTSPEMAKQVLKTHDIAFASRPKLLAMDIICYNRRDIAFSPYGEYWRQMRKICIMEVLSAKNVRSFSSIRHDEVVRLIDSIQPCFTSGELVNFTERIIWFTSSMTCRSAFGQVLKEQEVFIKLIREVISLAEGFDVADIFPSYKFLHGFGGAKKKLLNAHRKVDSIVEDVIKEHKKNIATRKSDDAIGGEDLVDVLLRLMNDKSLQFPINNDNIKAVIIDLFAAGTETSSTTTVWAMAEMLKNPSVFAKAQAEVREAFRDKVTFDGNDVEELKYLKLVIKETMRLHAPVPLLVPRECREETEINGYTIPVKTKVMVNVWALGRDPKYWDDAESFKPERFEQCSIDFIGNNFEYLPFGGGRRICPGISFGLANVYFPLAQLLYHFDWKLPTGMEPKDLDLTESAGITVARKGDLYLIATPHQP

SEQ ID NO: 30 - protein, 501 aa, Capsicum annuum
MQFFSLASIFLFLYFLFLLWKWNKSNSQTKKLPPGPWKLPFIGSMHHMAGGLPHYVLRDLAKKYGPFMHLQLGEVSAVIVTSSVVAKEVLKTHDLAFADRPKLLGMDIMCYDSRNIAFSPYGEYWRQMHKICIMELLSAKNIQSFSSIRHDEVVRLIDSIRSSSSSGELINYTKRIIWFTSSITCRSAFGKLLKDQEKFIKVVGEVTSLAQGFDVADIFPSYKFLHTFSGVKKKLLDAHRKVDSIVEDVINAHKRNIAAHKSDDTLGSEDLIDVLLRLAKDKSLKFPITNDNIKAVIVDMFAAGTETTSITTIWAMVEMMKNPSAFAKVQAEVREAFRDKATLDEMNVEELKYLKLVIKETMRIHPPLPLLIARECREETNINGYTIPENTKVIINAWALGRDPKYWDDAESFKPKRFEQCFMDFFGNNFEYLPFGSGRRICPGILFGLANVYLPLAQLLYHFDWKLPTGMEPRDLDMTESVGGTAPRKSDLYLVATPYQP

SEQ ID NO: 31 - protein, 521 aa, Solanum pennellii
MLDPSLSLSYLMEFQFTFNFIFLFLFLSFLYLLVVQRKKSRNLNKKLPPGPWKLPFIGSVHHLALEGGLPHHALTNLGKKYGPFMHLQLGEISTVVVSSMDMAREILKTHDLAFASRPKFLALDIICYKSTDIAFSPYGDYWRQMRKVCVLELLTTKNVRSFSSIRQDEASRLVQFIRSSTCGKPINVTERILWYQSSITCKAAFGELLKDQEKFIELVKELVELASGFSVADIFPSIKILHVLSGLRSRILKVHKNVDAIVEDTINEHKKNLASGKRGNGAFGGEDLVDVLLRLMESGELKIPITNDNIKAIMIDLFAAGSETSATTTIWAMTEMMRNPNVLAKAQAEVREAFKGKETFDEDVIEVLKYLKQIVKETLRLHPPVPLLVPRECREETNINGYTIPLKTRVMVNVYAMGRDPKYWEDAESFIPERFEHSSVDFMGNNFEYLPFGAGRRMCPGMSFGLINVYLPLAKLLYHFDWKLPHGLKPKDVDMTELSAITAARKSELYLIATPYYPSHE

SEQ ID NO: 32 - protein, 692 aa, Arabidopsis thaliana [sequence omitted]

SEQ ID NO: 33 - protein, 730 aa, Brassica napus
MSPPTVFLFYLSRVFRPLEREKISNPSQSQTKKKKMSSALYASGVFNEVKSILGAFLGDSVSDDVVLVIATTSLALVVGFVVLVWRKTTSDQSKELKPLVIPKSLMAKDEDEDVDLGSGKERVSIFFGTQTGTAEGFAKALAEEIKARYEKAAVKVIDLDDYAADDDQYEEKLKKETLSFFCVATYGDGEPTDNAARFYKWFTEGTEGDIRLQKLAYGVFALGNRQYEHFNKIGIVLDEELSKKGAKRLIEVGLGDDDQSIEDDFNAWKESLWPELDKLLRDEDDTSVATPYTAAIPEYRVMIHDPSFISEKSVDSSVANGNAAIDIHHPRRGNVAVQRELHTPESDRSCIHLEFDISCTGITYETGDHVGVYAENHAELVEEAAKLLGHTLDLVFSIHADKEDGSPLGSSLPPPFPGPCTLGTALAKYADLLTPPRKSALVALAAYATEPSEAKKLKHLTSPDGKDEYSQWIVASQRSLLEVMAAFPSAKPPLGVFFAAIAPRLQPRYYSISSSPRLAPNRVHVTCALVYGPTPTGRIHKGLCSTWMKNAVPAEKSRECSGAPIFIRASNFKLPSNPSTPIVMVGPGTGLAPFRGFLQERMALKEDGVELGPSLLFFGCRNRRMDFIYEDELNNYADQGVISELIVAFSREGAQKEYVQHKILEKATQVWNLIKEEGYLYVCGDAKGMARDVHRTLHTIVQEQEGVSSSEAEAIVKKLQTEGRYLRDVW

SEQ ID NO: 34 - protein, 694 aa, Tarenaya hassleriana
MSSSFGASGLVRYLESALGISLGDSVSDDAALVIATTSLAVIVGFAVLLWRKTAADRSREMKPVTVPQSLMAKDEDDDLDMGSGKTRVTIFFGTQTGTAEGFAKALTEEIKARYEKAAVKVVDMDDYAADDDQYAEKLKKETLAFFIIATYGDGEPTDNAARFYKWFTEGNERGAWLQQLTYGVFGLGNRQYEHFNKIGKVLDEQLSEQGAKRLVQVGLGDDDQSIEDDFNAWKEALWPELDKLLRDEDDTSVVSTYTAAIPEYRVFFYDPAVSPEEDSSSSMQNGSSVADIHHPFRVNVAVQKELHKPESDRSCIHLEFDISGTGITYETGDHVGVYAENHVETVGEAGKLLGQSLDLLFSVHADKEDGSPMGSSLPPPFPGPCTLHTALARYADLLNPPRKAALIALAAYATEPSEAEKLKHLTSPDGKDEYAQWIVASQRSLLEVMAAFPSAKPPLGVFFAAVAPRLQPRYYSISSSPRVAPNRVHVTCALVHGPTPTGRIHKGVCSTWMKNATPREKSDDCSAAPIFIRSSNFKLPSNPLTPIVMVGPGTGLAPFRGFLQERMALKENGVQLGLSLLFFGCRNRRMDFIYEDELYNFVDQGTLSEFVVAFSREGPQKEYVQHKMTEKAAEVWKLIKEGYLYVCGDAKGMARDVHRTLHTIVQEQEGVSSSEAEAIVKKLQNEGRYLRDVW

SEQ ID NO: 35 - protein, 694 aa, Quercus suber
MGSNSELVRTIESVLGISLGESVSDSVVLIVTTSFAVIIGLLVFVWKRSSDRSSSSLVKPVEVPKFSSSNKDEDDEVEVDSGKTKVSVFFGTQTGTAEGFAKALADEIKARYEKAVVKVIDLDDYAADDDQYEEKLKKETLAFFMVATYGDGEPTDNAARFYKWFTEGNDRGAWLQQLRYGVFGLGNRQYEHFNKIGNVVDEQLSEQGAKRLIPVGLGDDDQCIEDDFSAWREQLWPELDQLLLDGDDANTVSTPYTAVIPEYRVVIHDPIVTSYEDNYSNMANGTASFDIQHPCRVNVAVEKELHTPESDRSCIHLEFDISGTGITYETGDHVGVYAENCDETVEEAGKLLGQPLDLLFSIHTDNDDGTPLGSSLPPPFPGPCTLRAALARYADLLNPPRKAALIALAAHAVEPNEAERLKFLSSPQGKDDYSKWVVGSQRSLLEIMAEFPSAKPPLGVFFAAVAPRLPPRYYSISSSPRFAPNRIHVTCALVYGPTPTGRIHKGVCSTWMKNAVPLEKSRNCSWAPIFIRQSNFKLPADHSTPIIMVGPGTGFAPFRGFLQERMALKEEGVQLGPALLFFGCRNRRMDFIYEDELNNFVEQGVLSELIVAFSREGPQKEYVQHKMMDQAAHFWSLVSQGGYLYVCGDAKGMARDVHRTLHTIVQEQENVEPSKAEAIVKKLQMDGRYLRDVW

SEQ ID NO: 36 - protein, 692 aa, Prunus persica
MSSNSDLVSMLESALGVSFGGSVTDSVVVIATTSVALILGVLVLVWRRSSDRSREVKQLAVPKPLVVKDEEDEFEVASGKTRVTIFYGTQTGTAEGFAKALAEEIKARYEKAAVKVLDLDDYAEDDDKYEEKLKKETLAFFMLATYGDGEPTDNAARFYKWFTEGKDRGGWLQHLQYGVFALGNRQYEHFNKIGKVVDDQLSEEGAKRLIPVGLGDDDQCIEDDFSAWRESLWPELDQILRDEDDTNSVATPYTAVIPEYRVVIHDSTVVTSYEDNLLNMANGNASFDIHHPCRVNVAVQRELHKPESDRSCIHLEFDVSGTGITYETGDHVGVYADNCVETIEEAAKYLGQPLDLLFTLHTDSDDGTPLGSSLPPPFPGPCTLRTALAHYADLLNPPRKAALLALAAHAVEPSEAERLKFLSSSQGKDEYSKWIVGSQRSLVEVMAEFPSAKPPLGVFFAAVAPRLQPRYYSISSSPRFAPHRVHVTCALVYGPTPTGRVHKGVCSFWMKNAVSLEKSHNSSWAPIFIRQSNFKLPADPSVPVIMVGPGTGFAPFRGFLQERMALKKEGAQLGPALLFFGCRNRRMDFIYEDELNNFVDEGVLSELIVAFSREGPTKEYVQHKMMDRAAHMWDLISQGGYFYVCGDAKGMARDVHRTLHTIVQEQEKVDSTKAEAIVKQLQMDGRYLRDVW

SEQ ID NO: 37 - protein, 692 aa, Callitropsis nootkatensis [sequence omitted]

SEQ ID NO: 38 - protein, 589 aa, Pseudomonas fluorescens
MRSLKPLAALPDRLLDRLVHWARVRPEQTFIAAREAGGDWRLVSYGQMLDSVRAIAQGLLRYGLSADKPLALLSGNDIEHLQLALGAMYAGIPYCPVSPAYSLLSQDFAKLRHVCDLLQPGLVFVSDASAYQRAIDAVLPPETPLISVRGQVPGRRQASFASLLAEPGGAEADAAFAATGPDSIAKFLFTSGSTKLPKAVITTQRMLCANQQMLLQTFPVFGEEPPVLVDWLPWNHTFGGSHNVGIVLYNGGTFYLDEGKPTAQGFAETLRNLKEISPTAYLTVPKGWEELVNALEQDAELRECFFKRMSLFFFAAAGLSQSVWDRLDRVAEQHCGERIRMMAGLGMTEAAPSCTFTTGPLSMAGYIGLPAPGCEVRLVPVDGKLEGRFRGPHIMPGYWRAAQQTAEVFDAQGFYCSGDALKLADAADPQLGLMFDGRIAEDFKLSSGVFVSVGPLRNRAVLEGAPYVQDLVVAAPDRECLGALVFPRLYECRRLAGLGAEASEAQVLASAPVRQWFGDWLQCLNREASGNASRLEWIALQVEPASIDRGEITDKGSINQRAVLQWRAEQVENLYRGREPSILRAEPKP

SEQ ID NO: 39 - protein, 492 aa, Streptomyces sp. V-1
MVRNQGLGSWPVRRARMSPHATAVRHGGTALTYAELSRRVARLANGLRAAGVRPGDRVAYLGPNHPAYLETLFACGQAGAVFVPLNFRLGVPELDHALADSGASVLIHTPEHAETVAALAAGRLLRVPAGELDAADDEPPDLPVGLDDVCLLMYTSGSTGRPKGAMLTHGNLTWNCVNVLVETDLASDERALVAAPLFHAAALGMVCLPTLLKGGTVILHSAFDPGAVLSAVEQERVTLVFGVPTMYQAIAAHPRWRSADLSSLRTLLCGGAPVPADLASRYLDRGLAFVQGYGMTEAAPGVLVLDRAHVAEKIGSAGVPSFFTDVRLAGPSGEPVPPGEKGEIVVSGPNVMKGYWGRPEATAEVLRDGWFHSGDVATVDGDGYFHVVDRLKDMIISGGENIYPAEVENELYGYPGVEACAVIGVPDPRWGEVGKAVVVPADGSRIDGDELLAWLRTRLAGYKVPKSVEFTDRLPTTGSGKILKGEVRRRFG

SEQ ID NO: 40 - protein, 709 aa, Sphingomonas paucimobilis
MTVEAGVRPQAGARDINRLLRPRSIAIVGASETPGSLGASVLANLVRNEFPGDIHLVNPKRETISGRPAVPSVDALPEGVDCAILAIPRVAVLDTMRQLAARKAGAAIIFAAGFAEGGEQGMADQQEIGRIAHEAGIVVEGPNCLGSVNYLDRIPLTFIDTDIKAPSPGGVGIVSQSGAMAAVLAVMLESRTLDLTYSVSTGNEAGSGVEDYVEFMIADEKTRIIAMIVEQFRDPARFLALMDKANAAGKLVVLLHPGKSSAARESAATHTGAMAGDYKLMRAKVERAGVVVAETLEELGDITEIAARCPALPSGGTAVLGESGALKALTLDLAEELELALPTLDDGNAPALRAALPEFVPVSNPLDLTAQGLVDPDMYYRTLAALFEDDRVGTIFAGIIQTNPATIGIKLPPFLKAVRELKATKPVIFGGVDEGADVPADWIEQLRAEGIPYFPTTERALRAIRRLSAAGARDASRTDAAPASVPALASEKGVVPEYKAKALLAPLGISFPKGQFAATVEDAIAAAEAIGGPVVMKAQAAALSHKSDAGGVALNLVGAEAIRAAWDKMFADVKRYDASIILDGVLIEAMGARGLELIVGAKNDPQWGPVILAGFGGVTAEILQDVRLLSPDMTKEAIVAELGKLKQAALLHGYRGSPALDVGAVAELIGQVGRLLRGEPRIQELDLNPVVVYPEGQGAIALDALMLVD

SEQ ID NO: 41 - protein, 627 aa, Pseudomonas syringae
MSSEFRPQPRSGVAQTRYRHVSIGHSAVEVSERNGNLHMRSLEPLAPLPMRLLDRLVHWAQVRPLQTFIAAREQGGDWRRVSYAQMLDSVRAIAQSLLGYGLSAERPLAILSGNDIEHLQIALGAMYAGIPYCPVSPAYSLLSQDFAKLRHVCDLLQPGLIFVSDAAAYQRAIDAVLPTETPLVAVRGHVPGRSQVNFASLLEHPGGAEADVAFAATGPDSIAKFLFTSGSTKLPKAVITTQRMLCANQQMLLQTFPVFGEEPPVLVDWLPWNHTFGGSHNVGIVLYNGGTFYLDDGKPTAQGFAETLRNLKEVSPTAYLTVPKGWEELVSALENDGELRERFFAHMKLFFFAAAGLSQSVWDRLDQVAEQHCGERIRMMAGLGMTEASPSCTFTTGPLSMAGYIGLPAPGCEVRLVPVDGKLEGRFRGPHIMPGYWRAPQQTAEVFDDEGFYCSGDAIKLADPSDPQLGLMFDGRLAEDFKLSSGVFVSVGPLRNRAVLEGSPYVLDLVVAAPDRECLGALVFPRLYECRRLSGLTKAASDAEVLASAPVRAWFADWLQRLNREASGNASRLEWIAVLDEAASIDRGEITDKGSINQRAVLQWRAAKVEALYRGEDPSILRAGPRS

SEQ ID NO: 42 - protein, 499 aa, Nocardia amikacinitolerans
MRNRGLGSWPARRARMTPTAVAVTADDRQITYAQLRERVDRAAQVLRALGVGAGDRVAYLGPNHPVYLEILFAAGRLGAVFVPLNNRSAVAEIEYALTDSGARVLVHHCEHDAAVAALPGAAALGIVRVGGAGGYDDLLAAASAEPIEVEVGLDDDCLIMYTSGSTGRPKGAVLTHGNLTWNALNVLVESDVTGAEVSLVAAPLFHAAALGMICLPTLLKGGRVVLLAKFDPARVLELIERERVTWMFGVPTMFDALAAQPGWADADLSSLRILLCGGAPVPHATIARYVERGLGFVQGYGMTEAAPGVLVLDRAHAEAKLGSAGVPSFFTDVRIVDAEGAQVAPGERGEVLVSGPNVMRGYWNRPDATAEALRDGWFHSGDVATVDADGYAYIVDRVKDVIISGGENIYPAEVENEIAHHPQVAACAVIGVPDEKWGEVGRAVVVAREGTQPDADELLAFLRERLAGFKIPRSVVFVDELPTTGSGKIRKTEVKKQYG

SEQ ID NO: 43 - protein, 276 aa, Pseudomonas fluorescens
MSNYEGRWTTVKVEIEDGIAWVILNRPEKRNAMSPTLNREMIDVLETLEQDPAAGVLVLTGAGEAWTAGMDLKEYFREVDAGPEILQEKIRREASQWQWKLLRMYAKPTIAMVNGWCFGGGFSPLVACDLAICADEATFGLSEINWGIPPGNLVSKAMADTVGHRQSLYYIMTGKTFGGQKAAEMGLVNDSVPLARLREVTIELARNLLEKNPVVLRAAKHGFKRCRELTWEQNEDYLYAKLDQSRLLDTEGGREQGMKQFLDDKSIKPGLQAYKR

SEQ ID NO: 44 - protein, 287 aa, Streptomyces sp. V-1
MSTAVGNGRVRTEPWGETVLVEFDEGIAWVMLNRPDKRNAMNPTLNDEMVRVLDHLEGDDRCRVLVLTGAGESFSAGMDLKEYFREVDATGSTAVQIKVRRASAEWQWKRLANWSKPTIAMVNGWCFGGAFTPLVACDLAFADEDARFGLSEVNWGIPPGGVVSRALAATVPQRDALYYIMTGEPFDGRRAAEMRLVNEALPADRLRERTREVALKLASMNQVVLHAAKTGYKIAQEMPWEQAEDYLYAKLDQSQFADKAGARAKGLTQFLDQKSYRPGLSAFDPEK

SEQ ID NO: 45 - protein, 284 aa, Sphingomonas paucimobilis
MSEENKPVEREEDTVRYEIVNNVAWVYYNRPTKRNAQSPKLNRQMLKVLTELEFRDDVGVLVLGGEGPAWCAGMDLKEYFRETEAEGLAGTRKAQREAYTWWERLRWYQKPTIAMVHGWCFGGAYGPLFACDLAFAADEAQFGLSEVNWGILPGGGATKVAVDLMPMRVAMYHAMMGENLSGQDAARYNLVNESMPADQLKARVKQVAETLIQKNWATVKYTKDAVRRVKEMTYDNAEDYLIRLQEGLNWFDKSDGRHVAMKQFLDDKTFKPGLGHYDKTKTEV

SEQ ID NO: 46 - protein, 276 aa, Pseudomonas syringae
MSNYEGRWKTVKVEIEEGIAWVILNRPEKRNAMSPTLNREMIEVLEVLEQDPDAGVLVLTGAGEAWTAGMDLKEYFREVDAGPEILQEKIRREASQWQWKLLRMYAKPTIAMVNGWCFGGGFSPLVACDLAICADEATFGLSEINWGIPPGNLVSKAMADTVGHRQSLYYIMTGKTFGGQKAAELGLVNESVPLAQLRDVTVDLARNLLEKNPVVLRAAKQGFKRCRELTWEQNEDYLYAKLDQSRLLDTEGGREQGMKQFLDDKSIKPGLQTYKR

SEQ ID NO: 47 - protein, 278 aa, Saccharopolyspora flava
MSTSEKPWGNTVLVDFEDGIAWVTLNRPEKRNAMNPALNDEMVATLDALEADPRCRVLVLTGAGEAWSAGMDLKEYFREVDEADDPSVQIRVRRASAEWQWKRLANWSKPTIAMVNGWCFGGAFTPLVACDLAIADENARFGLSEINWGIPPGGVVTRALAATVPQRDALYYIMTGELFDGRQASEMRLVNEAVPAERLRERTRELAAKLAATNPVVLRAAKVGYKVARDMPWEQAEDYLYAKFEQSQFLDHTNGRQKGMSQFLDDKTYKPGLSAYRD

SEQ ID NO: 48 - protein, 356 aa, Vanilla planifolia
MAAKLLFFLLFLVSALSVALAGFEEDNPIRSVTQRPDSIEPAILGVLGSCRHAFHFARFARRYGKSYGSEEEIKKRFGIFVENLAFIRSTNRKDLSYTLGINQFADLTWEEFRTNRLGAAQNCSATAHGNHRFVDGVLPVTRDWREQGIVSPVKDQGSCGSCWTFSTTGALEAAYTQLTGKSTSLSEQQLVDCASAFNNFGCNGGLPSQAFEYVKYNGGIDTEQTYPYLGVNGICNFKQENVGVKVIDSINITLGAEDELKHAVGLVRPVSVAFEVVKGFNLYKKGVYSSDTCGRDPMDVNHAVLAVGYGVEDGIPYWLIKNSWGTNWGDNGYFKMELGKNMCGVATCASYPIVAV

SEQ ID NO: 49 - protein, 358 aa, Glechoma hederacea
MARLLLLLVGVLIACAAGARAGSEFLAEDNPIRQVVDGMHELESSILKAVGNSRRAFSFARFAHRYGKSYESSEEIQKRFQVYSENLRMIRSHNKKGLSYSMGVNEFSDLTWDEFKKHRLGAAQNCSATRRGNHKLTSAILPDSKDWRESGIVSPVKSQGSCGSCWTFSSTGALEAAYAQAFGKGISLSEQQLVDCAGAFNNFGCNGGLPSQAFEYIKYNGGLMTEEAYPYTGHDGECKYSSENAAVQVLDSVNITLGAEDELKHAVALVRPVSVAFEVVDGFRSYNGGVYTSTTCGSDPMDVNHAVLAVGYGVEGGVPYWLIKNSWGADWGDQGYFKMEMGKNMCGVATCASYPVVA

SEQ ID NO: 50 - protein, 525 aa, Saccharomyces pastorianus
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 51 - protein, 545 aa, Saccharomyces cerevisiae
METEESQFSSITKIINPKTLMNTYSEKTSLVQDECLVKMIQNGHSRRMGSVEDLYAALNRQKLYRNFSTYSELNDYCTKDQLALALRNICLKNPTLLHIVLPARWPDHKKYYLSSEYYSQPRPKHDYISVLPELKLDGVILNEQPEHNALMKQILEEFANSNGSYTAKIFKLTTALTIPYTGPTSPTWRLICLPEEDDTNKWKKFIFVSNHCMCDGRSSIHFFQDLRDELNNIKTLPKKLDYIFEYEKDYQLLRKLPEPIENMIDFRPPYLFIPKSLLSGFIYSHLRFSSKGVCTRMDEIEKSDEIVTEIINISPSEFQKIRTKIKLNIPGKCTITPFLEVCWFVTLHKWGKFFKPLKFEWLTDVFIPADCRSLLPEDEEVRAMYRYGANVGFVDFTPWISKFNMNDSKENFWPLIAHYHEVISGAIKDKKHLNGLGFNIQSLVQKYVNIDKVMRDRALGKSRGGTLLSNVGMFHQSEETEHKYRIRDLAFGQFQGSWHQAFSLGVSSTNVKGMNILISSTKNVVGSQELLEELCAMYKALLLNP

SEQ ID NO: 52 - protein, 524 aa, Saccharomyces kudriavzevii
MTKISEEHFPIQHECLERMIQNGHARRMGSVEDLYVALNRQKLYRNFSAYAELSDYCSKDQLTLALRNICLKNPTLLHIVLPTRWPDHENYYLSSEYYSHPHPEHDYISVLPELKLDGVIINEQPENGKIVRQILEEFRNSNGTYNAKIFKLTTALTIPYFGPTSPNWRLICLPEEHTDKWKKFIFVSNHCMSDGRSSIHFFHDLKAELNDIKTPPKKLDYLFKYENDYQLLRKLPEPIEKVIDFRPPYLFIPKSLLSGFIYNHLRFASRGICTRMDDMEKSDDVVAEIITISPSELQEIRTKIKSNIQGKCTLTPFLQVCWFVSLHQWGKFFKPLNFEWLTDIFIPADCRPQLPDDEEVRQMYRYGANVGFVDFTPWICESNMNDNKENFWPLIEHYHQVISGALRDNKHLHGLGLNIQGFVQKYVNIDKAMCDRAIGKARGGTLLSNVGMFKQLDSSNCNYSIKDLAFGQFQGSWHQAFSLGVCSTDVKGMNIIVASTKNVVGSQESLEELCRVYKAMLLGP

SEQ ID NO: 53 - protein, 535 aa, Saccharomyces cerevisiae
MEDIEGYEPHITQELIDRGHARRMGHLENYFAVLSRQKMYSNFTVYAELNKGVNKRQLMLVLKVLLQKYSTLAHTIIPKHYPHHEAYYSSEEYLSKPFPQHDFIKVISHLEFDDLIMNNQPEYREVMEKISEQFKKDDFKVTNRLIELISPVIIPLGNPKRPNWRLICLPGKDTDGFETWKNFVYVTNHCGSDGVSGSNFFKDLALLFCKIEEKGFDYDEEFIEDQVIIDYDRDYTEISKLPKPITDRIDYKPALTSLPKFFLTTFIYEHCNFKTSSESTLTARYSPSSNANASYNYLLHFSTKQVEQIRAQIKKNVHDGCTLTPFIQACFLVALYRLDKLFTKSLLEYGFDVAIPSNARRFLPNDEELRDSYKYGSNVGGSHYAYLISSFDIPEGDNDKFWSLVEYYYDRFLESYDNGDHLIGLGVLQLDFIVENKNIDSLLANSYLHQQRGGAIISNTGLVSQDTTKPYYVRDLIFSQSAGALRFAFGLNVCSTNVNGMNMDMSVVQGTLRDRGEWESFCKLFYQTIGEFASL

SEQ ID NO: 54 - protein, 545 aa, Saccharomyces eubayanus
MDSLEEYEPHVTQELIDRGHARRMGHLENYFAVLHRQNMYSNFAVYGELNKAVSKRQLRLALRLLLLKYPILSHTIVPKKYPNHEAYYLSQEYLNKPFPQHDFIKVIPHLELEDLIMNSQSEYREVISKVSEQYKNDNFKVTSSLIELVTPIIVPACDPERPNWRLICLPSRDTSAGEAWTNFVYVSNHCGSDGISGTNFFQDLTALLSTIEENGFDYEEEIVDNNVIIDYNEDHKEISKLPISITDRIDYKPRLTSLPKFFLKNIVSEYCEFRTSNESTVTSRYSPSSNANAGFNHLLHFSAEDTQQIRSEIKKKVHAGCTITPFIQACFFVALYRHDKVFTKSFLEYGFDVALPSSTRRFLPNDEELRDSYKYGANVGGSHYTYLISSFNVPEGDANKFWRLVEYYHDCFLKSYDNGDHLIGLGTLQLDFIVQNKNVDQIISTTYLDHQRGGAIISNTGFVRQDTTKPYYARDLIFSQSAGALRFAFGLNVCSTNVGGMNMDMSVVQGTLRNRGEWESFCDVFYHAIGEFASLGCDTAVAGLV

SEQ ID NO: 55 - protein, 571 aa, Artificial Sequence
  Arabidopsis thaliana 4-coumarate--coenzyme A ligase with an amino acid linker
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGLVDEAAAKSGR

SEQ ID NO: 56 - protein, 554 aa, Artificial Sequence
  Petroselinum crispum 4-coumarate--coenzyme A ligase with amino acid linker
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPKVDEAAAKSGR

SEQ ID NO: 57 - protein, 543 aa, Paulownia fortune
METTITKQEDIIFRSKLPDIYIPKHLPLHSYCFENISKFSTRPCLINGATNEVYTYEEVELIARKVATGLSKLGIQQGDTILLLLPNSPEYVFAFLGASYIGAISTMANPFFTPAEVIKQAKASNAKLIITQACYVEKVRDYALEKGVKVMCIDAPSADCLQFSELTSADERDMPAVKIHPDDVVALPYSSGTTGLPKGVMLTHKGLVTSVAQQVDGENPNLYIHSEDVMLCVLPLFHIYSLNSVLLCGLRVGAAILIMQKFDIVPFLELIQKYKVTIGPFVPPIVLAIVKSPVVDKYDLSSVRTVMSGAAPLGKELEDAVRIKFPNAKLGQGYGMTEAGPVLAMCLAFAKEPFEIKSGACGTVVRNAEMKIVDIETGASLGRNQPGEICIRGDQIMKGYLNDLESTEGTIDKDGWLHTGDIGFIDTDDELFIVDRLKEIIKYKGFQVAPAELEALLLNHPYISDAAVVPMKDEQAGEVPVAFVVRSNGSTITEDEIKQFISKQVVFYKRINRVFFIDAIPKSPSGKILRKDLRARLAAGVPN

SEQ ID NO: 58 - protein, 551 aa, Brassica napus
MAPQEDAMQKQSSNNSDVIFRSKLPDIYIPNHLPLHDYIFQNISEFASKPCLINGPTGHVYTYSDVHVASRRIAAGFQKLGVNKNDVVMILLSNCPEFVLSFLAASFRGATATAANPFFTPAEIAKQAKASNSKLIVTESRYVDKIKDLQNDGVIIVCTDEEPSPIPEGCLRFTELTQSTEIETVEISSDDVVALPYSSGTTGLPKGVMLTHRGLVTSVAQQVDGDNPNLYFHSDDVILCVLPLFHIYALNSIMLCGLRVGASILIMPKFEINLLLELIQRCKVTVAPMVPPIVLAMAKSPETEKYDLSSIRVVKSGAAPLGKELEDAVSAKFPNAKLGQGYGMTEAGPVLAMSLGFAKEPFPVKSGACGTVVRNAEMKIIDPDTGDSLSKNKPGEICIRGHQIMKGYLNNPAATAETIDKDGWLHTGDIGLIDDDDELFIVDRLKELIKYKGFQVAPAELEALLIGHQDITDVAVVAMKEEAAGEVPVAFVVKSKDSELSEDDVKQFVAKQVVFYKRINKVFFVESIPKAPSGKILRKDLRAKLVNGLVN

SEQ ID NO: 59 - protein, 545 aa, Capsicum baccatum 4
MPMENEAKQGDIIFRSKLPDIYIPNHLSLHSYCFENISEFSSRPCLINGANNQIYTYADVELNSRKVAAGLHKQFGIQQKDTIMILLPNSPEFVFAFLGASYLGAISTMANPLFTPAEVVKQVKASNAEIIVTQACHVNKVKDYALENDVKIVCIDSAPEGCVHFSELIQADEHDIPEVQIKPDDVVALPYSSGTTGLPKGVMLTHKGLVTSVAQQVDGENPNLYIHSEDVMLCVLPLFHIYSLNSVLLCGLRVGAAILIMQKFDIVPFLELIQNYKVTIGPFVPPIVLAIAKSPMVDNYDLSSVRTVMSGAAPLGKELEDTVRAKFPNAKLGQGYGMTEAGPVLAMCLAFAKEPFEIKSGACGTVVRNAEMKIVDPDTGNSLHRNQSGEICIRGDQIMKGYLNDPEATAGTIDKEGWLHTGDIGYIDNDDELFIVDRLKELIKYKGFQVAPAELEALLLNHPNISDAAVVPMKDEQAGEVPVAFVVRSNGSTITEDEVKEFISKQVIFYKRIKRVFFVDGVPKSPSGKILRKDLRAKLAAGFPN

SEQ ID NO: 60 - protein, 384 aa, Rheum palmatum
MATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCLAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 61 - protein, 383 aa, Rheum palmatum
MVTVEEVRKAQRAEGPATVLAIGTATPPNCVGQSTYPDYYFRITNSEHKIELKQKFQRMCDKSMIKKRYMYLTEEILKENPSMCEYMAPSLDARQDMVIVEIPKLGKEAATKAIKEWGQPKSKITHLVFCTTSGVDMPGADYQLIKLFGLRPSVKRLMMYQQGCFAGGTVLRLAKDLAENNRGARVLVVCSEITVVTFRGPSDTHLDCLVGQALFGDGVASIIVGADPLPEIEKPLFELVSAAQTILPDSEGAIEGHLREVGLTFHLLENVPALISKNIEKSLNETFKPLDIMDWNSLFWIAHPGGPAILDQVEAKLGLKPEKLEATGHILSEYGNMSSACVLFILDVVRRKSAANGVTTRILSIGQISKSLLILAWFLFSLV

SEQ ID NO: 62 - protein, 388 aa, Polygonum cuspidatum
MAASTDEMTKALTAATVLAIGTANPPNCYYQADFPDFYFRATNSDHLTHLKHKFKRICEKSMIEKRYLQLTEDILKENPNIGAYEAPSLDVRHEIQVKGVAQLGKEAALKAMQEWGQPKSKITHLIVCCIAGVDMPGANYQLTKLLDLNSSVKRFMFYHLGCYAGGTVLRLAKDIAENNKGARVLIVCSEMTPICFRGPSETHIDSMVGQAIFGDGAAAVIVGANPDLTVEEPIFELISTAQTIIPESDGAIEGHLLEVGLSFQLYQNVPALISNSIGTCLSEAFTPLNISNWNSLFWIAHPGGPAILDHVEATVGLNKEKLKATRQVLNDYGNMSSACVFFIMDEMRKKSLENGHATTGEGLQWGVLFGFGPGITVETVVLRSVPII

SEQ ID NO: 63 - protein, 389 aa, Camellia sinensis
MVTVEDIRRAQRAEGPATVMAIGTATPPNCVDQSTYPDYYFRITNSEHKAELKEKFKRMCDKSMIKKRYMYLTEEILKENPQVCGYMAPSLDARQDMVVVEVPKLGKEAATKAIKEWGQPKSKITHLVFCTTSGVDMPGADYQLTKLLGLRPSVKRLMMYQQGCFAGGTVLRLAKDLAENNKGARVLVVCSGITAVTFRGPSDTHLDSLVGQALFGDGAAAIIVGSDPIPEVENPLFELVSAAQTILPDSDGAIDGHLREVGLTFHLLKDVPGLISKNIEKSLAEAFQPLGISDWNSLFWIAHPGGPAILDQVELKLGLKEEKLRATRHVLSEYGNMSSACVLFILDEMRKKSAADGLKTTGEGLEWGVLFGFGPGLTVETVVLHSLCT

SEQ ID NO: 64 - protein, 393 aa, Vitis vinifera
MVSVGEIRKSQRAEGPATVLAIGTATPANCVYQADYPDYYFRITNSEHMTELKEKFKRMCEKSMINKRYMHLTEEILKENPNVCAYMAPSLDARQDMVVVEVPKLGKEAAVKAIKEWGQPKSKITHLVFCTTSGVDMPGADYQLTKLLGLKPSVKRLMMYQQGCFAGGTVLRLAKDLAENNAGARVLVVCSEITAVTFRGPSDTHLDSLVGQALFGDGAAAIIIGADPDTKIERPLFELVSAAQTILPDSEGAIDGHLREVGLTFHLLKDVPGLISKNIEKSLVEAFKPIGISDWNSLFWIAHPGGPAILDQVELKLGLKEEKLRATRHVLSEYGNMSSACVLFILDEMRKKSIEEGKGTTGEGLEWGVLFGFGPGLTVETVVLHSLATQSTH

SEQ ID NO: 65 - protein, 274 aa, Brettanomyces bruxellensis
MSFIGASRSIRGHIPIRALALSHSTSFKQPVRAIATYLHRTQHRPLYNTYKKGFSRNSFLLLAAMPLMTISDSVKDSLTKSEVVPTVIHDKSFLPKGFLTIQYDSGKEVALGNNIRPADSKNLPRIDFTLNLPSDASSTFNISKDDRFTLIVTDPDAPTRNDEKWSEYLHYLAVDVQLNTFNAENASSNDQLSTADLKGRTLYPYIGPGPPPKTGKHRYVFLLYKQTPGVTPEAPKDRPNWGTGIRGAGAAEYAEKYKLTPYAVNFFYAQNDQQ

SEQ ID NO: 66 - protein, 201 aa, Brettanomyces bruxellensis
MAKVAIIIYTLYHHISELAEAVKKGVEAAGSQADIFQVPETLPENILKILSAPKKPDYPIATTDTLTSYDAILFGVPTRFGNMPSQLKSFIDGTGGLWAKGALYHKPAGVFVSTNTGGGNEMTAVSLLSTFAHHGMIYVPLGFAKAFGELGTTSEAHGGSAWGAGCLAGADGSRTPSELELKIAHIQGEEFAKVAAQLTKN

SEQ ID NO: 67 - protein, 154 aa, Brettanomyces bruxellensis
MVKAVAVVRGDSTVKGVVTFEQTSESEPTTINYNIEGNDPNALRGFHIHTFGDNTNGCTSAGPHFNPFGKTHGAPTDENRHVGDLGNIKTDANGVAKGTIKDKLVKLIGANSIIGRTVVVHAGTDDLGKGGDAGSLQTGNAGGRPACGVIGLSA

SEQ ID NO: 68 - protein, 154 aa, Ogataea parapolymorpha
MVKAVAVVRGDSTVKGIVTFEQASESEPTTVSWEISGNDPNALRGFHIHQFGDNTNGCTSAGPHFNPFGKNHGAPEDSERHVGDLGNITTDANGVAKGAKQDSLIKLFGENSILGRTVVVHSGTDDLGKGGHPDSLKTGNAGGRPACGVIGFSS

SEQ ID NO: 69 - protein, 440 aa, Capsicum annuum
MAFALPSSLVSVCNKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDHTTTTLVPSPRFVGDSVFSTQKYGSLITPQILSDLNQCVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYKNVYTCSNLCKYPYYTVDFGWGRPERVCLGNGPSKNAFFLKDYKAGQGVEARVMLHKQQMSEFERNEELVEFIA

SEQ ID NO: 70 - protein, 440 aa, Capsicum frutescens
MAFALPSSLVSICDKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDHTTTTLVPSPRFVGDSVFSTKKYGSLITPQILSDLNECVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYENVYTCSNLCKYPYYTVDFGWGRPERVCLGNGPSKNAFFLKDYKAGQGVEARVMLHKQQMSEFERNEELLEFIA

SEQ ID NO: 71 - protein, 415 aa, Solanum lycospersicum
MAFALPSSLVSVCDKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDHTTTTLVPSPRFVGDSVFSTQKYGSLITPQILSDLNQCVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYENVYTCSNLCKYPYYTVDFGWGRPERVCLGNGPSKNAFFLKDYKAGQGV

SEQ ID NO: 72 - protein, 415 aa, Capsicum chacoense
MAFALPSSLVSVCDKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRINCSMSEILDHPHASLAESVVLPKDLPWANNCEGGNLLVVQVSKFDCGGIAISVCFSHKIGDGCSLLNFLNDWSSITRDHTTTTLVPSPRFVGDSVFSTQKYGSLITPQILSDLNECVQKRLIFPTDKLNALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYENVYTCSNLCKYPYYTVDFGWGRPERVCLGNGPSKNAFFLKDYKAGQGV

SEQ ID NO: 73 - protein, 440 aa, Capsicum annuum
MAFALPSSLVSVCNKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDRTTTTLVPSPRFVGDSVFSTQKYGSLITPQILSDLNQCVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYKNVYTCSNLCKYPYYTVDFGWGRPERVCLGNGPSKNAFFLKDYKAGQGVEARVMLHKQQMSEFERNEELLEFIA

SEQ ID NO: 74 - protein, 459 aa, Capsicum chinesne
MANITNEFMGHDMLAPFTAGWQSDMEPLVIEKSEGSYVYDINGKKYLDTLSGLWCATLGGSETRLVEAANKQLNTLPFYHSFWNRTTKPSLDLAKELLNMFTANKMAKVFFTNSGSEANDTQVKMVWYYNNALGRPQKKKIIARAKAYHGSTYISAGLSGLPPMHQKFDLPPPFVLHTECPHYWAYHLPGETEEEFSTRLANNLESLILKEGPETVAAFIAEPVLGAAGVILPPATYFDKVQAILRKHDILFIADEVVCGFGRLGTMFGSDKYNIKPDLVSVGKALSSGYMPIAAVLVSQKISSVILSESNKIGAFCHGFTYSGHPVACAVALEALKIYKERNITEVVNKISQKFQEGLKAFADSPIIGEIRGTGLALSTEFVDNKSPNDPFPYEWAVGTYFGAQCAKYGMLVSSTGDHVNMAPPFILSLEELDELIRIYGKALKDTEKRVEELKSQKQ

SEQ ID NO: 75 - protein, 459 aa, Capsicum frutesence
MANITNEFMGHDMLAPFTAGWQSDMEPLVIEKSKGSYVYDINGKKYLDTLSGLWCATLGGSETRLVEAANKQLNTLPFYHSFWNRTTKPSLDLAKELLNMFTANKMAKVFFTNSGSEANDTQVKLVWYYNNALGRPQKKKIIARAKAYHGSTYISAGLSGLPPMHQKFDLPPPFVLHTECPHYWAYHLPGETEEEFSTRLANNLESLILNEGPETVAAFIAEPVLGAAGVILPPATYFDKVQAILRKHDILFIADEVVCGFGRLGTMFGSDKYNIKPDLVSVAKALSSGYMPIAAVLVSQKISSVILSESNKIGAFCHGFTYSGHPVACAVALEALKIYKERNITEVVNKISQKFQEGLKAFADSPIIGEIRGTGLALSTEFVDNKSPNDPFPYEWAVGTYFGAQCAKYGMLVSSTGDHVNMAPPFTLSLEELDELIRIYGKALKDTEKRVEELKSQKK

SEQ ID NO: 76 - protein, 459 aa, Capsicum chinesnese
MANITNEFMGHDMLAPFTAGWQSDMEPLVIEKSEGSYVYDINGKKYLDTLSGLWCATLGGSETRLVEAANKQLNTLPFYHSFWNRTTKPSLDLAKELLNMFTANKMAKVFFTNSGSEANDTQVKLVWYYNNALGRPQKKKIIARAKAYHGSTYISAGLSGLPPMHQKFDLPPPFVLHTECPHYWAYHLPGETEEEFSTRLANNLESLILKEGPETVAAFIAEPVLGAAGVILPPATYFDKVQTILRKYDILFIADEVVCGFGRLGTMFGGDKYNIKPDLVSVAKALSSGYMPIAAVLVSQKISSVILSESNKIGAFCHGFTYSGHPVACAVALEALKIYKERNITEVVNKISQKFQEGLKAFADSPIIGEIRGTGLALSTEFVDNKSPNDPFPYEWAVGTYFGAQCAKYGMLVSSTGDHVNMAPPFMLSLEELDELIRIYGKALKDTEKRVEELKSQKK

SEQ ID NO: 77 - protein, 459 aa, Capsicum baccatum
MANITNEFMGHDMLAPFTAGWQSDMEPLVIEKSEGSYVYDINGKKYLDTLSGLWCTTLGGSETRLVEAANKQLNTLPFYHSFWNRTTKPSLDLAKELLNMFTANKMAKVFFTNSGSEANDTQVKLVWYYNNALGRPQKKKIIARAKAYHGSTYISAGLSGLPPMHQKFDLPPPFVLHTECPHYWAYHLPGETEEEFSTRLANNLESLILKEGPETVAAFIAEPVLGAAGVILPPATYFDKVQAILRKHDILFIADEVVCGFGRLGTMFGSDKYNIKPDLVSVAKALSSGYMPIAAVLVSQKISSVILSESNKIGAFCHGFTYSGHPVACAVALEALKIYKERNITEVVNKISQKFQEGLKAFADSPIIGEIRGTGLALSTEFVDNKSPNDPFPYEWAVGTYFGTQCAKYGMLVSSTGDHVNMAPSFTLSLEELDELIRIYGKALKDTEKRVEELKSQKK

SEQ ID NO: 78 - protein, 458 aa, Solanum lycopersicum
MAKTNGFMGHDMLAPFTAAWMIDMGPLVIDKAEGSYVYDVNGKKYLDSLSGLWCSVLGGSEPRLIEAANKQLNKLAFYHSFWNRTTKPSLDLAKELINMFTANKMGKVFFTSSGSEANDTQVKLVWYYNNAIGRPNKKKIISRKNAYHGSTYMTAGLSGLPSLHLKFDLPPPYILHTDCPHYWNYHLPGETEEEYSTRLANNLENLILKEGPETVAAFIAEPVMGGAGVIIPPATYFEKIQAVLKKYDILFIADEVICGFGRLGTMFGCDKYNIKPDLVSIAKALSGGYIPIGAVLVSEEISKVIMSQSNQLGVFCHGFTYSGHPVACAVALEALKIYKEKNITEVVNKLSPKFQEGLKAFIDSPIIGEIRGTGLVLSTEFVDNKSPNDPFPPEWGVGTYFGSQCQKHGMLVSFSGDHVNMAPPFTLSLEELDEMISIYGKALKDTEKRVEELKSQKK

SEQ ID NO: 79 - protein, 716 aa, Rhodosporidium toruloides
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 80 - protein, 505 aa, Arabidopsis thaliana
MDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 81 - protein, 954 aa, Artificial Sequence
  Chimeric polypeptide comprising Arabidopsis thaliana 4-coumarate-coenzyme A ligase and Rheum palmatum benzalacetone synthase
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGLVDEAAAKSGRATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCLAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 82 - protein, 937 aa, Artificial Sequence
  Chimeric polypeptide between Petroselinum crispum 4-coumarate--coenzyme A ligase and Rheum palmatum benzalacetone synthase
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPKVDEAAAKSGRATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCLAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 83 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 84 - protein, 425 aa, Petroselinum crispum
QLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPK

SEQ ID NO: 85 - protein, 10 aa, Artificial Sequence
  Amino acid linker
VDEAAAKSGR

SEQ ID NO: 86 - protein, 1887 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 87 - protein, 1887 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSCMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 88 - protein, 1887 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSAMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 89 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 90 - protein, 587 aa, Saccharomyces cerevisiae
MLWKRTCTRLIKPIAQPRGRLVRRSCYRYASTGTGSTDSSSQWLKYSVIASSATLFGYLFAKNLYSRETKEDLIEKLEMVKKIDPVNSTLKLSSLDSPDYLHDPVKIDKVVEDLKQVLGNKPENYSDAKSDLDAHSDTYFNTHHPSPEQRPRIILFPHTTEEVSKILKICHDNNMPVVPFSGGTSLEGHFLPTRIGDTITVDLSKFMNNVVKFDKLDLDITVQAGLPWEDLNDYLSDHGLMFGCDPGPGAQIGGCIANSCSGTNAYRYGTMKENIINMTIVLPDGTIVKTKKRPRKSSAGYNLNGLFVGSEGTLGIVTEATVKCHVKPKAETVAVVSFDTIKDAAACASNLTQSGIHLNAMELLDENMMKLINASESTDRCDWVEKPTMFFKIGGRSPNIVNALVDEVKAVAQLNHCNSFQFAKDDDEKLELWEARKVALWSVLDADKSKDKSAKIWTTDVAVPVSQFDKVIHETKKDMQASKLINAIVGHAGDGNFHAFIVYRTPEEHETCSQLVDRMVKRALNAEGTCTGEHGVGIGKREYLLEELGEAPVDLMRKIKLAIDPKRIMNPDKIFKTDPNEPANDYR

SEQ ID NO: 91 - protein, 500 aa, Saccharomyces cerevisiae
MTLPESKDFSYLFSDETNARKPSPLKTCIHLFQDPNIIFLGGGLPLKDYFPWDNLSVDSPKPPFPQGIGAPIDEQNCIKYTVNKDYADKSANPSNDIPLSRALQYGFSAGQPELLNFIRDHTKIIHDLKYKDWDVLATAGNTNAWESTLRVFCNRGDVILVEAHSFSSSLASAEAQGVITFPVPIDADGIIPEKLAKVMENWTPGAPKPKLLYTIPTGQNPTGTSIADHRKEAIYKIAQKYDFLIVEDEPYYFLQMNPYIKDLKEREKAQSSPKQDHDEFLKSLANTFLSLDTEGRVIRMDSFSKVLAPGTRLGWITGSSKILKPYLSLHEMTIQAPAGFTQVLVNATLSRWGQKGYLDWLLGLRHEYTLKRDCAIDALYKYLPQSDAFVINPPIAGMFFTVNIDASVHPEFKTKYNSDPYQLEQSLYHKVVERGVLVVPGSWFKSEGETEPPQPAESKEVSNPNIIFFRGTYAAVSPEKLTEGLKRLGDTLYEEFGISK

SEQ ID NO: 92 - protein, 513 aa, Saccharomyces cerevisiae
MTAGSAPPVDYTSLKKNFQPFLSRRVENRSLKSFWDASDISDDVIELAGGMPNERFFPIESMDLKISKVPFNDNPKWHNSFTTAHLDLGSPSELPIARSFQYAETKGLPPLLHFVKDFVSRINRPAFSDETESNWDVILSGGSNDSMFKVFETICDESTTVMIEEFTFTPAMSNVEATGAKVIPIKMNLTFDRESQGIDVEYLTQLLDNWSTGPYKDLNKPRVLYTIATGQNPTGMSVPQWKREKIYQLAQRHDFLIVEDDPYGYLYFPSYNPQEPLENPYHSSDLTTERYLNDFLMKSFLTLDTDARVIRLETFSKIFAPGLRLSFIVANKFLLQKILDLADITTRAPSGTSQAIVYSTIKAMAESNLSSSLSMKEAMFEGWIRWIMQIASKYNHRKNLTLKALYETESYQAGQFTVMEPSAGMFIIIKINWGNFDRPDDLPQQMDILDKFLLKNGVKVVLGYKMAVCPNYSKQNSDFLRLTIAYARDDDQLIEASKRIGSGIKEFFDNYKS

SEQ ID NO: 93 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 94 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 95 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 96 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 97 - protein, 386 aa, Saccharomyces cerevisiae
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 98 - protein, 382 aa, Saccharomyces cerevisiae
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 99 - protein, 351 aa, Saccharomyces cerevisiae
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

(19) United States
(12) Patent Application Publication
(10) Pub. No.: US 2022/0154113 A1
(43) Pub. Date: May 19, 2022

(54) EXPRESSION OF HETEROLOGOUS ENZYMES IN YEAST FOR FLAVOURED ALCOHOLIC BEVERAGE PRODUCTION

(71) Applicant: Danstar Ferment AG, Zug (CH)

(72) Inventors: Charles F. Rice, Plainfield, NH (US); Emily Agnes Stonehouse, Lebanon, NH (US); Nicholas Memmer, Enfield, NH (US); Jared Cameron Muysson, Minnetonka, MN (US); Bailey Morgan Carignan, Franklin, NH (US); Christopher J. Freeman, Sunapee, NH (US); Brooks Henningsen, Salisbury, NH (US); Hannah Lena Green, Sunapee, NH (US); Aaron Argyros, Lebanon, NH (US)

(21) Appl. No.: 16/978,461

(22) PCT Filed: Mar. 1, 2019
(86) PCT No.: PCT/IB2019/051682

§ 371 Date: Sep. 4, 2020

Related U.S. Application Data
(60) Provisional application No. 62/638,821, filed on Mar. 5, 2018.

(51) Int. Cl. C12C 12/00; C12N 9/04; C12N 15/52; C12N 1/18; C12P 7/56; C12P 7/62; C12P 7/26; C12C 11/00; C12G 3/02

(52) U.S. Cl. CPC C12C 12/006; C12N 9/0006; C12Y 101/01027; C12N 15/52; C12N 1/18; C12C 2200/05; C12P 7/62; C12P 7/26; C12C 11/003; C12C 12/004; C12G 3/02; C12P 7/56

(57) ABSTRACT

The present disclosure concerns recombinant yeast host cells expressing one or more heterologous polypeptide for making a flavour compound and a native ethanol production pathway. The recombinant yeast host cells can be used in a subsequent production process to make flavoured alcoholic beverage products, such as beers.

### TECHNOLOGICAL FIELD

The present disclosure relates to a recombinant yeast host cell in the production of flavoured alcoholic beverages, including those for the production of beer products.

### BACKGROUND

Introducing a flavour to an alcoholic beverage during its fermentation is technically challenging as it usually requires the use of additional microorganisms or the supplementation with purified flavour compound. One example of such flavoured alcoholic beverage is sour beer, a beer containing an appreciable amount of lactic acid as a flavoured compound. Sour beer fermentation has been carried out for centuries particularly within the regions of Germany and Belgium. These early fermentations employed spontaneous fermentation, a process by which the wort is inoculated by airborne wild microorganisms during the cooling process. This results in a complex consortium of microorganisms leading to a complexity of flavours, including sourness. The prevalent sour taste is provided from the accumulation of lactic acid, primarily produced by lactic acid bacteria (LAB) which are present in the wild inoculum. In recent years, sour beers have risen in popularity within the United States and several of the traditional methods have been revitalized for their production. These include the use of spontaneous fermentations using shallow open vats called “cool ships” to allow increased surface area for cooling wort and allowing natural microbiota to inoculate. But brewers have also modernized this approach by using controlled mixed fermentations inoculated from commercially available blends of yeasts and lactic acid bacteria, as well as “quick” or “kettle” souring where wort is initially fermented with LAB for approximately 24 to 48 hours prior to being boiled and inoculated with brewing yeast for primary fermentation. Alternatively, some wild, non-Saccharomyces yeast strains have been found to produce lactic acid in appreciable levels, thus providing a bacteria-free method of producing sour beer. Brewers can also use split fermentations in which the wort is fermented in separate vessels with normal brewing yeast for ethanol production and LAB or wild cultures for lactic production and then mixed together to achieve desired sourness.

Regardless of the souring method, all can be laborious and inherently difficult to control. Challenges include the need for a dedicated set of equipment to avoid contamination with non-sour beer fermentations, lack of reproducibility (of the spontaneous fermentation), organoleptic defects, long fermentation times and difficulty in propagating non-Saccharomyces yeast. Additionally, LAB and wild yeasts are a primary contaminant in traditional brewing processes and their use raises fear of contaminating non-sour batches within a brewery. This leads many brewers to utilize a dedicated set of equipment for sour beer production, further increasing capital costs and thus higher pricing of sour beer products. The kettle souring methodology has been employed by many commercial brewers as a means to increase both the speed and replicability of sour beer production. However, this method also contains drawbacks such as a newly created bottleneck within the brew kettle as new wort cannot be produced while the souring fermentation is underway, as well as the removal of volatile flavour compounds during the boiling step utilized to end the souring process. Using non-Saccharomyces strains for primary fermentation like Hanseniaspora vineae, Lachancea fermentati, Lachancea thermotolerans, Schizosaccharomyces japonicus and Wickerhamomyces anomalus are valid methods for lactic acidification, but these species behave differently and can ultimately result in a beer which has a different flavour profile than a Saccharomyces cerevisiae fermented beer. In addition, some of these strains may be difficult to propagate on an industrial scale and have been shown to ferment slowly compared to Saccharomyces cerevisiae.

There is thus a need to improve the consistency and reduce the variability in the production of flavoured and alcoholic beverages, such as the production of sour beers.

### BRIEF SUMMARY

The present disclosure relates to a recombinant yeast host cell capable of conducting an alcoholic fermentation (such as an anaerobic fermentation) to make an alcoholic beverage while producing a flavour compound. The recombinant yeast host cell expresses one or more heterologous proteins (e.g., enzymes) for producing the flavor compounds. The recombinant yeast host cell can be used in processes for making a flavoured and alcoholic beverage.

According to a first aspect, the present disclosure provides a recombinant yeast host cell for making a flavoured alcoholic beverage obtained by fermentation. The recombinant yeast host cell has an heterologous nucleic acid molecule encoding one or more heterologous polypeptide for the production of a flavour compound, wherein the heterologous nucleic acid molecule allows the production of the flavour compound. The recombinant yeast host cell has a native ethanol production pathway and can accumulate at least 5 g/L of ethanol during the fermentation.

In an embodiment, the flavour compound is/comprises lactic acid. In such embodiment, the one or more heterologous polypeptide comprises an enzyme having lactacte dehydrogenase (LDH) activity. In yet another embodiment, the enzyme having LDH activity is a LDH enzyme, a variant thereof or a fragment thereof. In still a further embodiment, the LDH enzyme is a Rhizopus oryzae LDH enzyme, a Sacchammyces cerevisiae LDH enzyme or a bovine LDH enzyme. In still another embodiment, the LDH enzyme is a Rhizopus oryzae LDH enzyme, a variant thereof or a fragment thereof. In yet a further embodiment, the LDH enzyme has the amino acid sequence of any one of SEQ ID NO: 2 to 11, is a variant of the amino acid sequence of any one of SEQ ID NO: 2 to 11, or is a fragment of the amino acid sequence of any one of SEQ ID NO: 2 to 11. In another embodiment, the enzyme having LDH activity is a mutated mitochondrial LDH enzyme for expression in the cytosol of the recombinant yeast host cell, a variant thereof or a fragment thereof. In a further embodiment, the mutated LDH enzyme is a mutated Saccharomyces cerevisiae mitochondrial LDH enzyme. In yet a further embodiment, the mutated Saccharomyces cerevisiae mitochondrial LDH enzyme is a DLD1 polypeptide and/or a CYB2 polypeptide. In another embodiment, the enzyme having LDH activity is a mutated malate dehydrogenase (MDH) enzyme capable of producing lactic acid, a variant thereof or a fragment thereof.

In an embodiment, the flavour compound is/comprises valencene. In such embodiment, the one or more heterologous polypeptide comprises a heterologous famesyl diphosphate synthase (FDPS) enzyme, a variant thereof or a fragment thereof. In another embodiment, the heterologous FDPS enzyme is a Arabidopsis thaliana FDPS enzyme, a Glycyrrhiza uralensis FDPS enzyme, a Capsella rubella FDPS enzyme or a Lupinus angustifolius FDPS enzyme. In another embodiment, the one or more heterologous polypeptide comprises a heterologous valencene synthase enzyme, a variant thereof or a fragment thereof. In yet a further embodiment, the heterologous valencene synthase enzyme is a Citrus sinensis valencene synthase enzyme, a Citrus junos terpene synthase enzyme, a Vitis vinifera valencene synthase enzyme, a Callitropsis nootkatensis valencene synthase enzyme or a Populus trichocarpa valencene synthase enzyme.

In an embodiment, the flavour compound is/comprises nootkatone. In such embodiment, the one or more heterologous polypeptide comprises a heterologous famesyl diphosphate synthase (FDPS) enzyme, a variant thereof or a fragment thereof. In another embodiment, the heterologous FDPS enzyme is a Arabidopsis thaliana FDPS enzyme, a Glycyrrhiza uralensis FDPS enzyme, a Capsella rubella FDPS enzyme or a Lupinus angustifolius FDPS enzyme. In a further embodiment, the one or more heterologous polypeptide comprises a heterologous valencene synthase enzyme, a variant thereof or a fragment thereof. In a further embodiment, the heterologous valencene synthase enzyme is a Citrus sinensis valencene synthase enzyme, a Citrus junos terpene synthase enzyme, a Vitis vinifera valencene synthase enzyme, a Callitropsis nootkatensis valencene synthase enzyme or a Populus trichocarpa valencene synthase enzyme. In another embodiment, the one or more heterologous polypeptide comprises a heterologous cytochrome P450 oxygenase enzyme, a variant thereof or a fragment thereof. In a further embodiment, the heterologous cytochrome P450 oxygenase enzyme is a Bacillus subtilis cytochrome P450 oxygenase enzyme, a Bacillus amyloliquefaciens cytochrome P450 enzyme, a Bacillus halotolerans cytochrome P450 enzyme, a Bacillus nakamurai cytochrome P450 enzyme or a Bacillus velezensis cytochrome P450 enzyme. In another embodiment, the one or more heterologous polypeptide comprises a heterologous cytochrome hydroxylase enzyme, a variant thereof or a fragment thereof. In a further embodiment, the heterologous cytochrome hydroxylase enzyme is a Hyoscyamus muticus cytochrome P450 hydroxylase enzyme, a Nicotiana attenuate cytochrome P450 hydroxylase enzyme, a Solanum tuberosum cytochrome P450 hydroxylase enzyme, a Capsicum annuum cytochrome P450 hydroxylase enzyme or a Solanum pennellii cytochrome P450 hydroxylase enzyme. In another embodiment, the one or more heterologous polypeptide comprises a heterologous cytochrome P450 reductase enzyme. In a further embodiment, the heterologous cytochrome P450 reductase enzyme is a Arabidopsis thaliana cytochrome P450 reductase enzyme, a Brassica napus cytochrome P450 reductase enzyme, a Tarenaya hassleriana P450 cytochrome reductase enzyme, a Quercus suber cytochrome P450 reductase enzyme or a Prunus persica cytochrome P450 reductase enzyme. In another embodiment, the one or more heterologous polypeptide comprises a heterologous valencene oxidase enzyme. In a further embodiment, the valencene oxidase enzyme is a Calitropsis nootkatensis valencene oxidase.

In an embodiment, the flavour compound is/comprises vanillin. In an embodiment, the one or more heterologous polypeptide comprises a heterologous feruloyl-CoA synthase (FCS) enzyme, a variant thereof or a fragment thereof. In a further embodiment, the heterologous FCS enzyme is a Pseudomonas fluorescens feruloyl-CoA synthetase enzyme, a Streptomyces sp. V-1 feruloyl-CoA synthetase enzyme, a Sphingomonas paucimobilis feruloyl-CoA synthetase enzyme, a Pseudomonas syringae feruloyl-CoA synthetase enzyme or a Nocardia amikacinitolerans feruloyl-CoA synthetase enzyme. In another embodiment, the one or more heterologous polypeptide comprises a heterologous enoyl-CoA hydratase (ECH) enzyme, a variant thereof or a fragment thereof. In a further embodiment, the heterologous ECH enzyme is a Pseudomonas fluorescens feruloyl-CoA synthetase enzyme, a Streptomyces sp. V-1 feruloyl-CoA synthetase enzyme, a Sphingomonas paucimobilis feruloyl-CoA synthetase enzyme, a Pseudomonas syringae feruloyl-CoA synthetase enzyme or Saccharopolyspora flava feruloyl-CoA hydratase. In an embodiment, the heterologous feruloyl-CoA synthetase enzyme has the amino acid sequence of SEQ ID NO: 38 or 39, is a variant of the amino acid sequence of SEQ ID NO: 38 or 39 or is a fragment of the amino acid sequence of SEQ ID NO: 38 or 39. In still another embodiment, the one or more heterologous polypeptide comprises a heterologous enoyl-CoA hydratase (ECH) enzyme, a variant thereof or a fragment thereof. For example, the heterologous enoyl-coA hydratase enzyme can be a Pseudomonas fluorescens feruloyl-CoA synthetase enzyme, a Streptomyces sp. V-1 feruloyl-CoA synthetase enzyme, a Sphingomonas paucimobilis feruloyl-CoA synthetase enzyme, a Pseudomonas syringae feruloyl-CoA synthetase enzyme or Saccharopolyspora flava feruloyl-CoA hydratase. In some embodiments, the heterologous enoyl-coA hydratase enzyme has the amino acid sequence of SEQ ID NO: 43 or 44, is a variant of the amino acid sequence of SEQ ID NO: 43 or 44 or is a fragment of the amino acid sequence of SEQ ID NO: 43 or 44. In a further embodiment, the recombinant yeast host cells lacks phenylacrylic acid decarboxylase enzymatic activity. In an embodiment, the one or more heterologous polypeptide comprises a heterologous vanillin synthase enzyme. In a further embodiment, the heterologous vanillin synthase enzyme is a Vanilla planifolia vanillin synthase enzyme or a Glechoma hederacea vanillin synthase enzyme.

In an embodiment, the flavour compound is/comprises isoamyl acetate. In an embodiment, the one or more heterologous polypeptide comprises a heterologous alcohol acetyl transferase (ATF) enzyme, a variant thereof or a fragment thereof. In a further embodiment, the heterologous ATF enzyme comprises a heterologous alcohol O-acetyltransferase (ATF1) enzyme. In still a further embodiment, the heterologous ATF1 enzyme is a Saccharomyces pastorianus ATF1 enzyme, a Saccharomyces cerevsiae ATF1 enzyme or a Saccharomyces kudriavzevii ATF1 enzyme. In a further embodiment, the heterologous ATF enzyme comprises a heterologous alcohol O-acetyltransferase (ATF2) enzyme. In some embodiments, the heterologous ATF2 enzyme is a Saccharomyces cerevisiae ATF2 enzyme or a Saccharomyces eubayanus ATF2 enzyme. In another embodiment, the recombinant yeast host cell overexpresses a native alcohol acetyl transferase (ATF) enzyme.

In an embodiment, the flavour compound is/comprises 4-(4-hydroxyphenyl)-2-butanone. In some embodiments, the one or more heterologous polypeptide comprises an heterologous phenylalanine-ammonium lyase (PAL) enzyme, a variant thereof or a fragment thereof. In some additional embodiments, the heterologous PAL enzyme is a Rhodosporidium toruloides PAL enzyme. For example, the heterologous PAL enzyme can have an amino acid sequence of SEQ ID NO: 79, be a variant of the amino acid sequence of SEQ ID NO: 79 or a be a fragment of SEQ ID NO: 79. In another embodiment, the one or more heterologous polypeptide comprises an heterologous cinnimate-4-hydroxylase (C4H) enzyme, a variant thereof or a fragment thereof. In some embodiments, the heterologous C4H enzyme is a Arabidopsis thaliana enzyme. For example, the heterologous C4H enzyme can have the amino acid sequence of SEQ ID NO: 80, be a variant of the amino acid sequence of SEQ ID NO: 80 or be a fragment of the amino acid sequence of SEQ ID NO: 80. In another embodiment, the one or more heterologous polypeptide comprises a heterologous coumarate-CoA ligase (4CL) enzyme, a variant thereof or a fragment thereof.

In a further embodiment, the heterologous 4CL enzyme is a Arabidopsis thaliana 4CL enzyme, a Petroselinum crispum 4CL enzyme, a Paulownia fortune enzyme, a Brassica napus 4CL enzyme, a Capsicum baccatum 4CL enzyme. For example, the 4CL enzyme can have the amino acid sequence of SEQ ID NO: 83 or 84, be a variant of the amino acid sequence of SEQ ID NO: 83 or 84 or be a fragment of the amino acid sequence of SEQ ID NO: 83 or 84. In another embodiment, the one or more heterologous polypeptide comprises a heterologous benzalacetone synthase (BAS) enzyme, a variant thereof or a fragment thereof. In a further embodiment, the heterologous BAS enzyme is a Rheum palmatum BAS enzyme, a Polygonum cuspidatum stilbene synthase enzyme, a Camellia sinensis chalcone synthase enzyme or a Vitis vinifera chalcone synthase enzyme. For example, the heterologous BAS enzyme can have the amino acid sequence of SEQ ID NO: 60, be a variant of the amino acid sequence of SEQ ID NO: 60 or be a fragment of the amino acid sequence of SEQ ID NO: 60. In some embodiments, the one or more heterologous polypeptide comprises a chimeric enzyme comprising an heterologous coumarate-CoA ligase (4CL) enzyme moiety and an heterologous benzalacetone synthase (BAS) enzyme moiety. For example, the chimeric enzyme can have the amino acid sequence of SEQ ID NO: 81 or 82, be a variant of the amino acid sequence of SEQ ID NO: 81 or 82 or be a fragment of the amino acid sequence of SEQ ID NO: 81 or 82. In some embodiment, the recombinant yeast host cell can overexpress a native benzalactone reductase.

In an embodiment, the flavour compound is/comprises 4-ethyl-phenol and/or 4-ethyl guiacol. In another embodiment, the one or more heterologous polypeptide comprises a heterologous vinylphenol reductase (VPR) enzyme, a variant thereof or a fragment thereof. In a further embodiment, the heterologous VPR enzyme is a Brettanomyces bruxellensis carboxypeptidase y enzyme, a Brettanomyces bruxellensis protoplast secreted protein 2 precursor polypeptide, a Brettanomyces bruxellensis superoxide dismutase or a Ogataea parapolymorpha superoxide dismutase.

In an embodiment, the flavour compound comprises phenylethyl alcohol. In such embodiment, the one or more heterologous polypeptide comprises a heterologous ARO8 enzyme, a variant thereof or a fragment thereof. In another embodiment, the one or more heterologous polypeptide comprises a heterologous ARO9 enzyme, a variant thereof or a fragment thereof. In still another embodiment, the one or more heterologous polypeptide comprises a heterologous PDC1 enzyme, a variant thereof or a fragment thereof. In yet another embodiment, the one or more heterologous polypeptide comprises a heterologous PDC5 enzyme, a variant thereof or a fragment thereof. In still another embodiment, the one or more heterologous polypeptide comprises a heterologous PDC6 enzyme, a variant thereof or a fragment thereof. In a further embodiment, the one or more heterologous polypeptide comprises a heterologous ARO10 enzyme, a variant thereof or a fragment thereof. In an embodiment, the one or more heterologous polypeptide comprises a heterologous SFA1 enzyme, a variant thereof or a fragment thereof. In yet a further embodiment, the one or more heterologous polypeptide comprises a heterologous ADH4 enzyme, a variant thereof or a fragment thereof. In still another embodiment, the one or more heterologous polypeptide comprises a heterologous ADH5 enzyme, a variant thereof or a fragment thereof.

In an embodiment, the flavour compound comprises ethyl capraote. In a further embodiment, the one or more heterologous polypeptide comprises a heterologous mutated FAS2 enzyme, a variant thereof or a fragment thereof.

In an embodiment, the flavour compound comprises vanillyloctanamide. In yet another embodiment, the one or more heterologous polypeptide comprises a heterologous capsaicin synthase enzyme, a variant thereof or a fragment thereof. In still a further embodiment, the one or more heterologous polypeptide comprises a heterologous pAMT1 enzyme, a variant thereof or a fragment thereof.

In an embodiment, the heterologous nucleic acid molecule is operatively associated with a promoter which can be a native or an heterologous promoter. In an embodiment, the promoter is the heterologous promoter and comprises a promoter from the tef2 gene, the cwp2 gene, the ssa1 gene, the eno1 gene, the eno2 gene, the hxk1 gene, the pgk1 gene, the hxt7 gene, the hxt3 gene, the dan1 gene, the gdp1 gene, the gpd2 gene, the ssu1 gene, the ssu1-r gene, the pau5 gene, the hor7 gene, the adh1 gene, the tdh1 gene, the tdh2 gene, the tdh3 gene, the cdc19 gene, the pdc1 gene and/or the tpi1 gene. In a further embodiment, the heterologous promoter is the promoter from the adh1 gene.

In an embodiment, the heterologous nucleic acid molecule is operatively associated with a terminator which can be a native or an heterologous terminator. In an embodiment, the terminator is the heterologous terminator and comprises the terminator from the dit1 gene, the adh3 gene, the idp1 gene, the gpm1 gene, the pma1 gene, the tdh3 gene, the hxt2 gene, the cyc1 gene, the pgk1 gene, and/or the ira2 gene.

In an embodiment, the recombinant yeast host cell is from the genus Saccharomyces sp. In a further embodiment, the recombinant yeast host cell is from the species Saccharomyces cerevisiae or Saccharomyces pastonrianus.

In yet a further embodiment, the fermentation is an anaerobic fermentation.

According to a second aspect, the present disclosures provides a fermenting agent for making a flavoured and fermented alcoholic beverage comprising or consisting essentially of the recombinant yeast host cell described herein. In an embodiment, the fermenting agent further comprising a nutrient.

According to a third aspect, the present disclosure provides a combination for making a flavoured and fermented alcoholic beverage comprising or consisting essentially of the recombinant yeast host cell described herein and a non-genetically modified yeast.

According to a fourth aspect, the present disclosure provides a process for making a flavoured and fermented alcoholic beverage, the process comprising (i) contacting the recombinant yeast host cell, the fermenting agent or the combination described herein with substrate comprising carbohydrates to provide a mixture and (ii) fermenting the mixture so as to accumulate the flavor compound and at least 5 g/L of ethanol in the fermented mixture. In an embodiment, the carbohydrates of the substrate comprise a majority of maltose and maltotriose. In yet another embodiment, the fermenting step is conducted under anaerobic conditions. In another embodiment, the flavoured and fermented alcoholic beverage is beer, mead, brandy, whisky, rum, vodka, gin, or tequila. In yet a further embodiment, the flavoured and fermented alcoholic beverage is beer.

According to a fourth aspect, the present disclosure provides a process for making a beer, the process comprising (i) contacting the recombinant yeast host cell, the fermenting agent or the combination described herein with a substrate comprising carbohydrates to provide a mixture and (ii) fermenting the mixture so as to accumulate the flavor compound and at least 5 g/L of ethanol in the fermented mixture. In an embodiment, the carbohydrates of the substrate comprise a majority of maltose and maltotriose and can be, for example, a wort. In some embodiments, the process further comprises at least one of: providing or making the wort; conducting a secondary fermenting step after step (ii); conducting a filtering step after step (ii); or conducting a sterilizing step after step (ii). In yet another embodiment, the fermenting step is conducted under anaerobic conditions. In an embodiment, the beer is a sour beer. In yet another embodiment, the sour beer comprises at most 3.0% w/v lactic acid.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows lactic acid profiles of parent M14629 ale strain and lactate dehydrogenase transformant, strain M16141, in a lab-scale ale fermentation. Results are shown as the concentration of lactic acid (in g/L) in function of time (in hours) and the strains or combination of strains used (♦=M14629 only, ▪=M16141 only, ▴=a 50:50 combination of M14629 and M16141 orX=a 25:75 combination of M14629 and M16141).

FIG. 2 shows specific gravity profiles of parent M14629 ale strain and lactate dehydrogenase transformant, strain M16141 in a lab-scale ale fermentation. Results are shown as the specific in function of time (hours) and the strains or combination of strains used (♦=M14629 only, ▪=M16141 only, ▴=a 50:50 combination of M14629 and M16141 or X=a 25:75 combination of M14629 and M16141).

FIG. 3 shows ethanol production profiles of parent M14629 ale strain and lactate dehydrogenase transformant, strain M16141, in a lab-scale ale fermentation. Results are shown as the concentration of ethanol (in g/L) in function of time (in hours) and the strains or combination of strains used (♦=M14629 only, ▪=M16141 only, ▴=a 50:50 combination of M14629 and M16141 orX=a 25:75 combination of M14629 and M16141).

FIG. 4 shows isoamyl acetate profiles of parent M14635 ale strain and alcohol O-acetyltransferase (ATF1) transformant, strain M16626, in a lab-scale ale fermentation. Results are shown as the concentration of isoamyl acetate (mg/L) in function of time (hours).

FIG. 5 compares the lactic acid production (g/L) in parental ale strain M14629, strain M16141 (expressing the heterologous R. oryzae lactate dehydrogenase) and M18231 (expressing the heterologous L. fermentati lactacte dehydrogenase) when grown in YPD media.

FIG. 6 compares the vanillin (in ppm, left axis, dark gray bars) and ferulic acid (ppm, right axis, light gray bars) production in parental strain M2390, strain M16872 (expressing the heterologous P. fluorescens FCS and ECH) and strain M16873 (expressing the heterologous Streptomyces sp. FCS and ECH) when grown in YPD media supplemented with 2 000 ppm of ferulic acid.

FIG. 7 compares the vanillin production (in ppm) in parental strain M14629 as well as M17807 during a beer fermentation (dry malt extract) supplemented with 2 000 ppm ferulic acid.

FIG. 8 compares the raspberry ketone production (in ppb) in parental strain M14629 as well as strain M17735 and M17736 during a beer (dry mal extract) fermentation.

FIG. 9 shows lactic acid profiles of co-fermentations with parent M14629 ale strain and lactate dehydrogenase transformant, strain M16141, in a lab-scale ale fermentation (13° Plato wort fementation at 20° C.). Results are shown as the concentration of lactic acid (in g/L) in function of time (in days) (♦ M14629 only; ▪ 80% M14629 and 20% M16141; ▴ 70% M14629 and 30% M16141; X 60% M14629 and 40% M16141; [symbol] 50% M14629 and 50% M16141; ● 40% M14629 and 60% M16141; +30% M14629 and 70% M16141; ◯ 20% M14629 and 80% M16141; Δ 100% M16141).

FIG. 10 shows ethanol profiles of co-fermentations with parent M14629 ale strain and lactate dehydrogenase transformant, strain M16141, in a lab-scale ale fermentation (13° Plato wort fementation at 20° C.). Results are shown as the concentration of ethanol (in g/L) in function of time (in days) (♦ M14629 only; ▪ 80% M14629 and 20% M16141; ▴ 70% M14629 and 30% M16141; X 60% M14629 and 40% M16141; [symbol] 50% M14629 and 50% M16141; ● 40% M14629 and 60% M16141; +30% M14629 and 70% M16141; ◯ 20% M14629 and 80% M16141; Δ 100% M16141).

FIG. 11 shows lactic acid profiles of parent M14629 ale strain and lactate dehydrogenase transformants with varying promoters: strains M16141 (adh1p), M16868 (dan1p), M16869 (tdh1p), and M16869 (tpi1p) in lab-scale ale fermentation (13° Plato wort fementation at 20° C.). Results are shown as the concentration of lactic acid (in g/L) in function of time (in hours).

FIG. 12 shows ethanol profiles of parent M14629 (dashed line) ale strain and lactate dehydrogenase transformants with varying promoters: strains M16141 (adh1p ▴), M16868 (dan1p ▪), M16869 (tdh1p ⋄), and M16869 (tpi1p ●) in lab-scale ale fermentation (13° Plato wort fementation at 20° C.). Results are shown as the concentration of ethanol (in g/L) in function of time (in hours).

FIG. 13 shows lactic acid profiles of parent M13175 (dashed line) lager strain and lactate dehydrogenase transformant M16394 (adh1p regular line) in lab-scale ale fermentation (13° Plato wort fementation at 10° C.). Results are shown as the concentration of lactic acid (in g/L) in function of time (in hours).

FIG. 14 shows ethanol profiles of parent M13175 (dashed line) lager strain and lactate dehydrogenase transformant M16394 (adh1p regular line) in lab-scale ale fermentation (13° Plato wort fementation at 10° C.). Results are shown as the concentration of ethanol (in g/L) in function of time (in hours).

FIG. 15 shows lactic acid production at 120 h for the parent M13175 lager strain and lager lactate dehydrogenase transformant M16394 (adh1p), along with the ale strain parent M14629 with the corresponding LDH transformant M16141 (adh1p) in lab-scale ale fermentation (13° Plato wort fementation at 20° C.). Results are shown as the concentration of lactic acid (in g/L) at the final time point 120 h.

FIG. 16 shows ethanol production at 120 h for the parent M13175 lager strain and lager lactate dehydrogenase transformant M16394 (adh1p), along with the ale strain parent M14629 with the corresponding LDH transformant M16141 (adh1p) in lab-scale ale fermentation (13° Plato wort fementation at 20° C.). Results are shown as the concentration of ethanol (in g/L) at the final time point 120 h.

### DETAILED DESCRIPTION

The present disclosure provides recombinant yeast host cells expressing one or more heterologous polypeptides (and in an embodiment, one or more heterologous enzymes) for the production of a flavour compound. As used herein, a “flavour compound” refers to compounds capable of triggering a flavour sensation in humans. In some embodiments of the present disclosure, the production of a flavour compound occurs during the conversion of a substrate, such as a carbohydrate substrate, into biomass (e.g., the fermentation). During the fermentation, at least a portion of a carbohydrate substrate is utilized/converted by the biomass to make both the flavour compound (e.g., to at least a minimal level and/or up to a maximal level) and ethanol (to at least a minimal level). The present disclosure provides for a recombinant yeast host cell capable of producing the flavour compound in the fermentation medium, so as to accumulate a minimal and/or maximal amount of the flavor compound in the fermentation medium once the carbohydrates have been converted (e.g., after the conversion of the carbohydrates). As used herein, the “conversion of the carbohydrates” or the “carbohydrates have been converted” is achieved when at least 50%, at least 60%, at least 70%, at least 80%, at least 90%, at least 95%, at least 99%, or at least 99.9% of the carbohydrate substrate is utilized by the yeast biomass. The “conversion of carbohydrates” or “carbohydrates have been converted” can also be achieved when a certain level of ethanol is produced in the fermentation medium, for example when at least 1%, 2%, 3%, 4%, 5%, 6%, 7%, 8%, 9%, 10% v/w 12%, 14%, 16%, 18%, 20%, 25%, 30%, 35%, 40% v/w or more of ethanol is produced in the fermentation medium. In some embodiments, the “conversion of carbohydrates” or “carbohydrates have been converted” is achieved when a certain level of carbohydrates remains in the fermentation medium, for example when at most 15, 14, 13, 12, 11, 10, 9, 8, 7, 6, 5, 4, 3, 2 or 1 g/L of carbohydrates remain in the fermentation medium.

In the context of the present disclosure, the production of the flavour compound and ethanol usually occurs during the fermentation and, in an embodiment, simultaneously during the fermentation. In some embodiments, the production of the flavour compound occurs more rapidly in fermentation when compared to a traditional method (for example, lactic acid production from a bacterial fermentation of a brewing medium). In an embodiment, the substrate of the fermentation medium or mixture include fermentable materials which contain C6 sugar as for example fructose, glucose, galactose, sucrose, maltose or starch, as well as their degradation products. As an example, the fermentable material can comprise be a fruit (apple, grape, pears, plums, cherries, peaches), a plant (sugar cane, agava, cassava, ginger), a sugar material (honey, molasse), a starchy material (rice, rye, corn, Sorghum, millet, barley, wheat, potatoes) or a derived product (grape must, apple mash, malted grain, crushed fruit, fruit puree, fruit juice, fruit must, plant mash, gelatinized and saccharified starch from different plant origins as rice, corn, sorghum, wheat, barley). In another embodiment, the substrate of the fermentation medium or mixture can be or comprise a starchy material. In the context of the present disclosure, a “starchy material” refers to a material that contains starch that could be converted into alcohol by a yeast during alcoholic fermentation. Starchy material could be for example, gelatinized and saccharified starch from cereals, grains (wheat, barley, rice, buckwheat) or grain derived-products (malted grain or a wort) or vegetable (potatoes, beets). In yet another embodiment, the fermentation medium can be or comprise, but is not limited to, malt, barley, wheat, rye, oats, corn, buckwheat, millet, rice, or sorghum. In a specific embodiment, the fermentation medium comprises, as the majority (e.g., major source) of carbohydrates, maltose and maltotriose. In such embodiments, other carbohydrates, such as glucose or fructose can be present, but in a lesser amount than maltose and maltotriose. In such embodiment, the recombinant yeast host cell of the present disclosure is able to metabolize efficiently maltose and maltotrise, especially when they are provided as the majority (e.g., major source) of carbohydrates in the fermentation medium. Further, the recombinant yeast host cell can be obtained from a brewing or a distilling yeast parental cell. In some embodiments, the fermentation medium excludes a fermentation medium having, as the majority (e.g., major source) of carbohydrates, glucose and fructose, such as, for example a grape or apple must (e.g., a wine must). In some embodiments, the recombinant yeast host cell of the present disclosure usually lacks the ability to sporulate or form spores for sexual reproduction. Alternatively, the recombinant yeast host cell lacks the ability to sporulate or form spores for sexual reproduction. In an embodiment, the recombinant yeast host cell usually does not produce a killer protein. Alternatively, the recombinant yeast host cell does not produce a killer protein. In a further embodiment, the recombinant yeast host cell cannot be obtained from a wine yeast parental cell.

The propagated biomass comprising the recombinant yeast host cell can be used in a fermenting step (usually under anaerobic conditions) to allow the production of the desired metabolites (e.g., a flavoured compound and ethanol). In some embodiments, a monoculture of the recombinant yeast host cell is used as the sole fermenting/flavouring organism to make the flavoured and alcoholic beverage. In another embodiment, the recombinant yeast host cell of the present disclosure is used in combination with another fermenting/flavouring organism (which, in some embodiments, could include additional yeasts or fungi and, in further embodiment, could include bacteria) to make the flavoured and alcoholic beverage. The recombinant yeast host cells can advantageously be easily measured, dosed and formulated for ease of use in downstream operations. As such, the recombinant yeast host cells improve the consistency and reduce the variability in the production of flavoured and alcoholic beverages.

### Recombinant Yeast Host Cells

The recombinant yeast host cells of the present disclosure are intended to be used for making flavoured and alcoholic beverages for human consumption. In preferred embodiments, the recombinant yeast host cells of the present disclosure are used in a fermentation process (such as, for example, an anaerobic fermentation process). The fermentation process can be followed by a distillation process to make distilled spirits.

The recombinant yeast host cells of the present disclosure can be provided in an active form (e.g., liquid (such as, for example, a cream yeast), compressed, or fluid-bed dried yeast), in a semi-active form (e.g., liquid, compressed, or fluid-bed dried), in an inactive form (e.g., drum- or spray-dried) as well as a mixture thereof. In an embodiment, the recombinant yeast host cells are provided in an active and dried form.

The present disclosure concerns recombinant yeast host cells that have been genetically engineered. The genetic modification(s) is(are) aimed at increasing the expression of a specific targeted gene (which is considered heterologous to the yeast host cell) and can be made in one or multiple (e.g., 1, 2, 3, 4, 5, 6, 7, 8 or more) genetic locations. The genetic modification(s) is(are) also aimed at decreasing or removing the expression of a specific targeted gene (which is considered native to the yeast host cell) and can be made in one or multiple (e.g., 1, 2, 3, 4, 5, 6, 7, 8 or more) genetic locations. In the context of the present disclosure, when recombinant yeast cell is qualified as being “genetically engineered”, it is understood to mean that it has been manipulated to add at least one or more heterologous or exogenous nucleic acid residue. In some embodiments, the one or more nucleic acid residues that are added can be derived from an heterologous cell or the recombinant yeast host cell itself. In the latter scenario, the nucleic acid residue(s) is (are) added at one or more genomic location which is different than the native genomic location. The genetic manipulations did not occur in nature and are the results of in vitro manipulations of the yeast. The genetic modification(s) in the recombinant yeast host cell of the present disclosure comprise, consist essentially of or consist of a genetic modification allowing the expression of an heterologous nucleic acid molecule encoding for one or more heterologous polypeptide for the production of a flavour compound. In the context of the present disclosure, the expression “a genetic modification allowing the expression of an heterologous nucleic acid molecule encoding for one or more heterologous polypeptide for the production of a flavour compound” refers to the fact that the recombinant yeast host cell can include other genetic modifications which are unrelated to the anabolism or the catabolism of the flavour compound or ethanol.

When expressed in a recombinant yeast host cells, the heterologous polypeptides described herein can be encoded on one or more heterologous nucleic acid molecules. The term “heterologous” when used in reference to a nucleic acid molecule (such as a promoter, a terminator or a coding sequence) or a protein/polypeptide refers to a nucleic acid molecule or a protein/polypeptide that is not natively found in the recombinant host cell. “Heterologous” also includes a native coding region/promoter/terminator, or portion thereof, that was removed from the source organism and subsequently reintroduced into the source organism in a form that is different from the corresponding native gene, e.g., not in its natural location in the organism's genome. The heterologous nucleic acid molecule is purposively introduced into the recombinant yeast host cell. For example, a heterologous element could be derived from a different strain of host cell, or from an organism of a different taxonomic group (e.g., different kingdom, phylum, class, order, family genus, or species, or any subgroup within one of these classifications). As used herein, the term “native” when used in inference to a gene, polypeptide, enzymatic activity, or pathway refers to an unmodified gene, polypeptide, enzymatic activity, or pathway originally found in the recombinant host cell. In some embodiments, heterologous polypeptides derived from a different strain of host cell, or from an organism of a different taxonomic group (e.g., different kingdom, phylum, class, order, family genus, or species, or any subgroup within one of these classifications) can be used in the context of the present disclosure.

The heterologous nucleic acid molecule present in the recombinant host cell can be integrated in the recombinant yeast host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a recombinant yeast host cell are well known in the art and include homologous recombination. The heterologous nucleic acid molecule can be present in one or more copies (e.g., 2, 3, 4, 5, 6, 7, 8 or even more copies) in the recombinant yeast host cell's genome. Alternatively, the heterologous nucleic acid molecule can be independently replicating from the recombinant yeast host cell's genome. In such embodiment, the nucleic acid molecule can be stable and self-replicating.

In the context of the present disclosure, the recombinant host cell is a yeast and in some embodiments the yeast can be used in the production of alcoholic beverages. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula, Hanseniaspora, Lachancea, Wickerhamomyces or Yarrowia. Suitable yeast species can include, for example, S. cerevisiae, S. bulderi, S. bametti, S. exiguus, S. uvarum, S. diastaticus, C. utilis, K. lactis, K. marxianus K. fragilis, Hanseniaspora vineae, Lachancea fermentati, Lachancea thenmotolerans, Schizosaccharomyces japonicus and/or Wickerhamomyces anomalus. In some embodiments, the yeast is selected from the group consisting of Saccharomyces cerevisiae, Schizzosaccharomyces pombe, Candida albicans, Pichia pastoris, Pichia stipitis, Yarrowa lipolytica, Hansenula polymorpha, Phatlia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiment, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the host cell can be an oleaginous microalgae host cell (e.g., for example, from the genus Thraustochytrium or Schizochytrium). In an embodiment, the recombinant yeast host cell is from the genus Saccharomyces and, in some embodiments, from the species Saccharomyces cerevisiae. In some embodiments, the recombinant Saccharomyces cerevisiae can be obtained from a brewing strain of Saccharomyces cerevisiae. For example, the recombinant Saccharomyces sp. can be obtained from strain of Saccharomyces sp. capable of metabolizing a medium comprising, as a majority of carbohydrates, maltose and maltotriose. For example, the Saccharomyces strain can be a brewing strain. In the context of the present disclosure, a brewing strain refers to a yeast strain capable of producing an alcoholic beer. Brewing strains include, without limitations, ale strains (such as, for example, a Saccharomyces cerevisiae strain) and a lager strains (such as, for example, a Saccharomyces pastorianus strain)). In some embodiments, the brewing strain can be obtained from a strain of Saccharomyces sp. which usually reproduce using asexual reproduction or budding (for example a non-sporulating Saccharomyces sp. strain). Alternatively, the brewing strain can be obtained from a strain of Saccharomyces sp. which only reproduces using asexual reproduction or budding (for example a non-sporulating Saccharomyces sp. strain). In another embodiment, the brewig strain is capable of metabolizing a fermenting a medium comprising, as the majority of the carbohydrates, maltose and maltotriose. In still another embodiment, the brewing strain is obtained from a Saccharomyces sp. strain which usually fails to produce a killer protein. Alternatively, the brewing strain is obtained from a Saccharomyces sp. strain which fails to produce a killer protein. In some embodiments, the recombinant Saccharomyces sp. can be obtained from a distilling strain of Saccharomyces sp. In the context of the present disclosure, a brewing strain refers to a yeast strain capable of producing a fermented medium that can be used in the preparation of a distilled alcohol. In some embodiments, the distilling strain can be obtained from a strain of Saccharomyces sp. which usually reproduce using asexual reproduction or budding (for example a non-sporulating Saccharomyces sp. strain). Alternatively, the distilling strain can be obtained from a strain of Saccharomyces sp. which only reproduces using asexual reproduction or budding (for example a non-sporulating Saccharomyces sp. strain). In another embodiment, the distilling strain is capable of metabolizing a fermenting a medium comprising, as the majority of the carbohydrates, maltose and maltotriose. In still another embodiment, the distilling strain is obtained from a Saccharomyces sp. strain which usually fails to produce a killer protein. Alternatively, the distilling strain is obtained from a Saccharomyces sp. strain which fails to produce a killer protein.

The present disclosure concerns recombinant yeast host cells having the intrinsic ability to make a minimal amount of ethanol suitable in the manufacture of an alcoholic beverages by fermentation. For example, the recombinant yeast host cells can express one or more polypeptide (which can be endogenous/native or heterologous) in an ethanol production pathway in order to achieve a minimal amount of ethanol during or after the fermentation. In some embodiments, the minimal amount of ethanol is at least 5 g/L, 10 g/L, 20 g/L, 30 g/L, 40 g/L, 50 g/L or more during or after fermentation (but prior to distillation, if any), or after at least partial conversion of the carbohydrate substrate into its metabolites. In one embodiment, the minimal amount of ethanol is 5 g/L. The recombinant yeast host cell of the present disclosure may have a native (e.g., not genetically modified) and functional ethanol production pathway to allow it to reach the minimal ethanol level during fermentation. Enzymes involved in ethanol production include, but are not limited to, pyruvate decarboxylase (PDC), alcohol dehydrogenase (ALD), lactate dehydrogenase (LDH), glucokinase, glucose-6-phosphate isomerase, phosphofructokinase, aldolase, triosephosphate isomerase, glyceraldehyde 3-phosphate dehydrogenase, 3-phosphoglycerate kinase, phosphoglycerate mutase, enolase, pyruvate kinase, pyruvate decarboxylase and/or alcohol dehydrogenase.

However, in some embodiments, the recombinant yeast host cell of the present disclosure may be genetically modified to increase the activity of one or more polypeptide in the ethanol production pathway so as to reach the minimal ethanol level. In one embodiment, the recombinant yeast host cells can have a modified/heterologous promoter to increase expression of one or more polypeptide in the ethanol production pathway. In another embodiment, the recombinant yeast host cells have a heterologous nucleic acid molecule encoding one or more heterologous polypeptide in the ethanol production pathway. The polypeptides involved in the ethanol production pathway include, but are not limited to pyruvate decarboxylase(s) (PDC), alcohol dehydrogenase(s) (ALD), mitochondrial lactate dehydrogenase (CYB2 and/or DLD1) as well as the enzymes involved in glycolysis (for example those listed in Table 1). In an embodiment, the recombinant yeast host cell of the present disclosure comprises at least one genetic modification to increase the expression of at least one of the following enzymes: pyruvate decarboxylase (PDC), alcohol dehydrogenase (ALD), lactate dehydrogenase (LDH), glucokinase, glucose-6-phosphate isomerase, phosphofructokinase, aldolase, triosephosphate isomerase, glyceraldehyde 3-phosphate dehydrogenase, 3-phosphoglycerate kinase, phosphoglycerate mutase, enolase, pyruvate kinase, pyruvate decarboxylase and/or alcohol dehydrogenase. In an embodiment, the recombinant yeast host cell of the present disclosure comprises a combination of more than one genetic modification to increase the expression of more than one of the following enzymes: pyruvate decarboxylase (PDC), alcohol dehydrogenase (ALD), lactate dehydrogenase (LDH), glucokinase, glucose-6-phosphate isomerase, phosphofructokinase, aldolase, triosephosphate isomerase, glyceraldehyde 3-phosphate dehydrogenase, 3-phosphoglycerate kinase, phosphoglycerate mutase, enolase, pyruvate kinase, pyruvate decarboxylase and/or alcohol dehydrogenase.

TABLE 1
Primary genes involved in glycolysis

| Gene | Enzyme |
|---|---|
| GLK1, HXK1, HXK2 | glucokinase |
| PGI1 | glucose-6-phosphate isomerase |
| PFK1, PFK2 | phosphofructokinase |
| FBA1 | aldolase |
| TPI1 | triosephosphate isomerase |
| TDH1, TDH2, TDH3 | glyceraldehyde 3-phosphate dehydrogenase |
| PGK1 | 3-phosphoglycerate kinase |
| GPM1 | phosphoglycerate mutase |
| ENO1, ENO2 | enolase |
| PYK2, CDC19 | pyruvate kinase |
| PDC1, PDC5, PDC6 | pyruvate decarboxylase |
| ADH1, ADH2, ADH3, ADH4, | alcohol dehydrogenase |
| ADH5 | |

In an embodiment, the recombinant yeast host cell of the present disclosure includes (and in an embodiment expresses) a nucleic acid molecule coding for a pyruvate decarboxylase. The pyruvate decarboxylase may be native or heterologous to the recombinant yeast host cell and includes, but is not limited to, fungal, plant, bacterial, yeast, or other microorganism derived pyruvate decarboxylase. In one embodiment, the pyruvate decarboxylase is derived from the PDC1, PDC5, and/or PDC6 gene. In one embodiment, the pyruvate decarboxylase is derived from the PDC1 and PDC5 genes, the PDC5 and PDC6 genes, or the PDC1 and PDC6 genes. In one embodiment, the pyruvate decarboxylase is of the PDC1, PDC5, and PDC6 genes. In another embodiment, the pyruvate decarboxylase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for an alcohol dehydrogenase. The alcohol dehydrogenase may be native or heterologous to the recombinant yeast host cell and includes, but is not limited to, fungal, plant, bacterial, yeast, or other microorganism derived alcohol dehydrogenase. In an embodiment, the alcohol dehydrogenase is derived from the ADH1, ADH2, ADH3, ADH4, and/or ADH5 genes. In another embodiment, the alcohol dehydrogenase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a glucokinase. The glucokinase may be native or heterologous to the recombinant yeast host cell and includes, but is not limited to, fungal, plant, bacterial, yeast, or other microorganism derived glucokinase. In one embodiment, the glucokinase is derived from the GLK1, HXK1, or HXK2 gene. In one embodiment, the glucokinase is derived from the GLK1 and HXK1 genes, the HXK1 and HXK2 genes, or the GLK1 and HXK2 genes. In one embodiment, the glucokinase is derived from the GLK1, HXK1, and HXK2 genes. In another embodiment, the glucokinase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a glucose-6-phosphate isomerase. The glucose-6-phosphate isomerase may be native or heterologous to the recombinant yeast host cell and includes, but is not limited to, fungal, plant, bacterial, yeast, or other microorganism derived glucose-6-phosphate isomerase. In one embodiment, the glucose-8-phosphate isomerase is derived from the PGI1 gene. In another embodiment, the glucose-8-phosphate isomerase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a phosphofructokinase. The phosphofructokinase may be native or heterologous to the recombinant yeast host cell and includes, but is not limited to, fungal, plant, bacterial, yeast, or other microorganism derived phosphofructokinase. In one embodiment, the phosphofructokinase is derived from the PFK1 and/or PFK2 gene. In another embodiment, the phosphofructokinase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for an aldolase. The aldolase may be native or heterologous to the recombinant yeast host cell and includes, but are not limited to, fungal, plant, bacterial, yeast, or other microorganism derived aldolase. In one embodiment, the aldolase is of the FBA1 gene. In another embodiment, the aldolase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a triosephosphate isomerase. The triosephosphate isomerase may be native or heterologous to the recombinant yeast host cell and includes, but is not limited to, fungal, plant, bacterial, yeast, or other microorganism derived triosephosphate isomerase. In one embodiment, the triosephosphate isomerase is of the TPI1 gene. In one embodiment, the aldolase is of the FBA1 gene. In another embodiment, the triosephosphate isomerase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a glyceraldehyde 3-phosphate dehydrogenase. The glyceraldehyde 3-phosphate dehydrogenase may be native or heterologous to the recombinant yeast host cell and includes, but is not limited to, fungal, plant, bacterial, yeast, or other microorganism derived glyceraldehyde 3-phosphate dehydrogenase. In one embodiment, the glyceraldehyde 3-phosphate dehydrogenase is derived from the TDH1, TDH2, or TDH3 gene. In one embodiment, the glyceraldehyde 3-phosphate dehydrogenase is derived from the TDH1 and TDH2 genes, TDH2 and TDH3 genes, or TDH1 and TDH3 genes. In one embodiment, the glyceraldehyde 3-phosphate dehydrogenase is derived from the TDH1, TDH2, and TDH3 genes. In another embodiment, the glyceraldehyde 3-phosphate dehydrogenase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a 3-phosphoglycerate kinase. The 3-phosphoglycerate kinase may be native or heterologous to the recombinant yeast host cell and includes, is are not limited to, fungal, plant, bacterial, yeast, or other microorganism derived 3-phosphoglycerate kinase. In one embodiment, the 3-phosphoglycerate kinase is derived from the PGK1 gene. In another embodiment, the glyceraldehyde 3-phosphoglycerate kinase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a phosphoglycerate mutase. The phosphoglycerate mutase may be native or heterologous to the recombinant yeast host cell and includes, but is not limited to, fungal, plant, bacterial, yeast, or other microorganism derived phosphoglycerate mutase. In one embodiment, the phosphoglycerate mutase is derived from the GPM1 gene. In another embodiment, the phosphoglycerate mutase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for an enolase. The enolase may be native or heterologous to the recombinant yeast host cell and includes, but is not limited to, fungal, plant, bacterial, yeast, or other microorganism derived enolase. In one embodiment, the enolase is derived from the ENO1, and/or ENO2 gene. In another embodiment, the enolase is from a yeast, for example from the species Saccharomyces and in a further embodiment from Saccharomyces cerevisiae.

In an embodiment, the recombinant yeast host cell of the present disclosure includes (and in an embodiment expresses) a nucleic acid molecule coding for a pyruvate kinase. The pyruvate kinase may be native or heterologous to the recombinant yeast host cell and includes, but is not limited to, fungal, plant, bacterial, yeast, or other microorganism derived pyruvate kinase. In one embodiment, the pyruvate kinase is of the PYK2, and/or CDC19 gene. In another embodiment, the enolase is from a yeast, for example from the species Saccharomyces and in a further embodiment from Saccharomyces cerevisiae.

The recombinant yeast host cell of the present disclosure includes an heterologous nucleic acid molecule encoding one or more heterologous polypeptide for the production of at least one or a combination of flavour compound(s), such as, for example, those listed in Table 2. As such, the recombinant yeast host cells of the present disclosure is intended to express, at least during the fermentation process for making the flavoured alcoholic beverage, one or more heterologous polypeptide for making at least one flavour compound. However, in some embodiments, in order to avoid organoleptic defects in the alcoholic beverage, care must be taken to as to limit the production of the one or more flavour compounds to a maximal amount. For example, in embodiments in which the flavor compound should not exceed a specific threshold (e.g., lactic acid for example), the recombinant yeast host cell can be used to provide a maximal amount of the flavour compound produced during fermentation which can be at most about 3.0, 2.9. 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.09, 0.08, 0.07, 0.06, 0.05, 0.04, 0.03, 0.02 or 0.01 w/v percent with respect to the weight of the alcoholic mixture after fermentation. In such embodiments, the recombinant yeast host cell can also be used to provide a minimal detectable amount of the flavour compound which is going to depend on the type of alcoholic beverage produced. In yet another example, in embodiments in which the flavor compound should met a minimal threshold (such as, for example, valencene, nootkatone, vanillin, isoamyl acetate, 4-(4-hydroxyphenyl)-2-butanone, 4-ethyl-phenol and 4-ethyl guiacol, phenylethyl alcohol and/or ethyl capraote, vanillyloctanamide), the recombinant yeast host cell can be used to provide a minimal amount of the flavor compound produced during fermentation which can be at least about 0.1, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900, 1 000 ppb or more. In still another example, in embodiments in which the flavor compound should met a minimal threshold (such as, for example, valencene, nootkatone, vanillin, isoamyl acetate, 4-(4-hydroxyphenyl)-2-butanone, 4-ethyl-phenol and 4-ethyl guiacol, phenylethyl alcohol and/or ethyl capraote, vanillyloctanamide), the recombinant yeast host cell can be used to provide a minimal amount of the flavor compound produced during fermentation which can be at least about 0.1, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900, 1 000 ppm or more. In one embodiment, the maximal amount or minimal amount of flavour compound the recombinant yeast host cells can produce during fermentation depends on the type of flavour compound and/or the type of alcoholic beverage. A list of embodiments of the flavour compounds is provided in Table 2, together with example gene expression modification in a recombinant host yeast cell for the production of the flavour compounds. A list of the detectable amounts of flavour compound for the embodiments of flavour compounds (from Table 2) is provided in Table 3.

In some embodiments, the recombinant yeast host cell of the present disclosure can be further modified to delete and/or upregulate the expression of one or more native genes for the production of at least one or a combination of flavour compound(s), such as, for example, those listed in Table 2. As such, the recombinant yeast host cells of the present disclosure is intended to express, at least during the fermentation process for making the flavoured alcoholic beverage, one or more heterologous polypeptide for making at least one flavour compound.

In some embodiments, recombinant yeast host cell of the present disclosure includes an heterologous nucleic acid molecule encoding one or more heterologous polypeptide and is modified to delete and/or upregulate one or more native genes for the production of at least one or a combination of flavour compound(s), such as, for example, those listed in Table 2. As such, the recombinant yeast host cells of the present disclosure is intended to express, at least during the fermentation process for making the flavoured alcoholic beverage, one or more heterologous polypeptide for making at least one flavour compound.

TABLE 2
Flavours, genes, and pathways involved in
production of flavour compounds

| | | | Native |
|---|---|---|---|
| | Flavour | Genes for expression or | genes for |
| Flavour | compound | overexpression | deletion |
| Sour | lactic acid | lactate dehydrogenase | N.A. |
| Citrus (orange) | valencene | farnesyl diphosphate | N.A. |
| | | synthase, | |
| | | valencene synthase | |
| | | 3-hydroxy-3-methylglutaryl- | |
| | | coenzyme A reductase 1 | |
| | | (HMG1) | |
| Citrus | nootkatone | farnesyl diphosphate | N.A. |
| (Grapefruit) | | synthase, | |
| | | valencene synthase, | |
| | | valencene oxydase | |
| | | cytochrome P450 | |
| | | monooxygenase | |
| | | cytochrome P450 | |
| | | hydroxylase | |
| | | cytochrome P450 reductase | |
| | | 3-hydroxy-3-methylglutaryl- | |
| | | coenzyme A reductase 1 | |
| | | (HMG1) | |
| Vanilla | vanillin | feruloyl-CoA synthetase, | Δ PAD1 |
| | | feruloyl-CoA hydratase | |
| Banana | isoamyl acetate | alcohol acetyl transferase | N.A. |
| | | (ATF1 and/or ATF2) | |
| Raspberry | 4-(4- | phenylalanine-ammonium | N.A. |
| | hydroxyphenyl)- | lyase | |
| | 2-butanone | cinnimate-4-hydroxylase | |
| | | coumarate-CoA ligase | |
| | | benzalacetone synthase | |
| Brettanornyces | 4-ethyl-phenol | vinylphenol reductase | N.A. |
| flavours | and 4-ethyl | | |
| | guiacol | | |
| Rose-like | phenylethyl | ARO8 | |
| | alcohol | ARO9 | |
| | | ARO10 | |
| | | PDC1 | |
| | | PDC5 | |
| | | PDC6 | |
| | | SFA1 | |
| | | ADH4 | |
| | | ADH5 | |
| Green apple | ethyl capraote | Mutant FAS2 | N.A. |
| Spicy | vanillyloctanamide | capsaicin synthase | N.A. |
| | | pAMT1 | |

TABLE 3
Embodiments of detectable of flavour
compounds produced by the recombinant
yeast host cells during fermentation
(depending on the alcholic beverage)

| Flavour compound | Detectable | |
|---|---|---|
| | Amount | |
| lactic acid | 1.0 to 3.0 | g/L |
| valencene | N/A | |
| nootkatone | 0.8-1 | ppb |
| vanillin | 20-200 | ppb |
| isoamyl acetate | 250-300 | ppb |
| 4-(4-Hydroxyphenyl)-2-butanone | 100 | ppb |
| 4-ethyl-phenol | 50 | ppb |
| 4-ethyl guiacol | 50 | ppb |

The heterologous enzymes listed in Table 2 are examples, and other heterologous enzymes derived from a different strain of host cell, or from an organism of a different taxonomic group (e.g., different kingdom, phylum, class, order, family genus, or species, or any subgroup within one of these classifications) can be used. In some embodiments, the recombinant yeast host cell of the present disclosure includes one or more heterologous nucleic acid molecule encoding one or more heterologous polypeptide for the production of one or more flavour compound, including one or more of the flavour compounds listed in Table 2 and combinations thereof. In an embodiment, the recombinant yeast host cell is genetically modified to make a single flavour compound from the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, or vanillyloctanamide. In still another embodiment, the recombinant yeast host cell is genetically modified to make at least two flavour compounds from any combinations of the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In still a further embodiment, the recombinant yeast host cell is genetically modified to make at least three flavour compounds from any combinations of the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In yet another embodiment, the recombinant yeast host cell is genetically modified to make at least four flavour compounds from any combinations of the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In another embodiment, the recombinant yeast host cell is genetically modified to make at least five flavour compounds from any combinations of the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In a further embodiment, the recombinant yeast host cell is genetically modified to make at least six flavour compounds from any combinations of the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In still yet another embodiment, the recombinant yeast host cell is genetically modified to make at least seven flavour compounds from any combinations of the following list: lactic acid, valencene, nootkatone, vanillin, isoamyl acetate, 4-(4-Hydroxyphenyl)-2-butanone, 4-vinyl-phenol, 4-vinyl guiacol, ethyl capraote, phenylethyl alcohol, and/or vanillyloctanamide. In still yet another embodiment, the recombinant yeast host cell is genetically modified to make at least eight flavour compounds from any combinations of the following list: lactic acid, valencene, nootkatone, vanillin, isoamyl acetate, 4-(4-Hydroxyphenyl)-2-butanone, 4-vinyl-phenol, 4-vinyl guiacol, ethyl capraote, phenylethyl alcohol, and/or vanillyloctanamide. In still yet another embodiment, the recombinant yeast host cell is genetically modified to make at least nine flavour compounds from any combinations of the following list: lactic acid, valencene, nootkatone, vanillin, isoamyl acetate, 4-(4-Hydroxyphenyl)-2-butanone, 4-vinyl-phenol, 4-vinyl guiacol, ethyl capraote, phenylethyl alcohol, and/or vanillyloctanamide. In still yet another embodiment, the recombinant yeast host cell is genetically modified to make at least ten flavour compounds from any combinations of the following list: lactic acid, valencene, nootkatone, vanillin, isoamyl acetate, 4-(4-Hydroxyphenyl)-2-butanone, 4-vinyl-phenol, 4-vinyl guiacol, ethyl capraote, phenylethyl alcohol, and/or vanillyloctanamide. In still another embodiment, the recombinant yeast host cell is genetically modified to make all the flavour compounds from the following list: lactic acid, valencene, nootkatone, vanillin, isoamyl acetate, 4-(4-Hydroxyphenyl)-2-butanone, 4-ethyl-phenol, 4-ethyl guiacol, ethyl capraote, phenylethyl alcohol, and/or vanillyloctanamide. In an embodiment, the recombinant yeast host cell does not include a genetic modification for making 4-(4-hydroxyphenyl)-2-butanone. In a further embodiment, when the recombinant yeast host cell is obtained from a wine strain, the recombinant yeast host cell does not include a genetic modification for making 4-(4-hydroxyphenyl)-2-butanone.

In an embodiment, the recombinant yeast host cell of the present disclosure includes (and in an embodiment expresses) a nucleic acid molecule coding one or more heterologous polypeptide for the production of lactic acid. As used in the present disclosure, the term “lactate dehydrogenase” (LDH) refers to a polypeptide capable of the enzyme classification 1.1.1.27 and capable of catalyzing the conversion of lactate to pyruvic acid and/or pyruvic acid into lactate.

In one embodiment, the enzyme having LDH activity is an heterologous LDH enzyme. For example, the one or more polypeptide for the production of lactic acid can comprise lactate dehydrogenase from a Rhizopus sp. (such as for example, from a Rhizopus oryzae), a variant thereof or a fragment thereof. In some embodiments, the Rhizopus oryzae lactate dehydrogenase is encoded by the nucleotide molecule having the sequence of SEQ ID NO: 1 (or a variant thereof or a fragment thereof). In other embodiment, the Rhizopus oryzae lactate dehydrogenase has the amino acid sequence of SEQ ID NO: 2 (or a variant thereof or a fragment thereof). In some embodiments, the heterologous lactate dehydrogenase is derived from the Lachancea sp. (for example from Lanchancea fermentati (which can have, for example, the amino acid sequence of SEQ ID NO: 3, 4, 5 6 or 10 a variant thereof or a fragment thereof) or Lachancea thermotolerans (which can have, for example, the amino acid sequence of SEQ ID NO: 8 or 9, a variant thereof or a fragment thereof)) or from the Wickerhamomyces sp. (for example from Wickerhamomyces anomalus and can have, for example, the amino acid sequence of SEQ ID NO: 11, a variant thereof or a fragment thereof).

In some embodiments, the recombinant yeast host cell is genetically engineered to redirect the expression of a mitochondrial LDH enzyme to the cytosol. In such embodiment, the native gene encoding for the mitochondrial LDH enzyme can be mutated in the recombinant yeast host cell. Alternatively or in combination, an heterologous nucleic acid molecule coding for a mutated LDH enzyme (which can be expressed and localized in the cytosol) can be introduced in the recombinant yeast host cell. As such, the recombinant yeast host cell can comprise an heterologous nucleic acid coding for a mutated mitochondrial LDH enzyme that can localize to the cytosol. In an embodiment, the heterologous nucleic acid includes a gene coding for a mitochondrial LDH enzyme lacking a mitochondrial signal sequence which, upon expression, will provide the mitonchondrial enzyme in the cytosol. In an embodiment, the genes encoding the mitochondrial lactate dehydrogenase (LDH) enzymes that can be mutated include, but are not limited to, the DLD1 gene and/or the CYB2 gene. For example, the mitochondrial LDH enzyme can be a mutant of the S. cerevisiae DLD1 enzyme having the amino acid sequence of SEQ ID NO: 90, a variant thereof or a fragment thereof. In another example, the mitochondrial LDH enzyme can be a mutant of the S. cerevisiae CYB2 enzyme having the amino acid sequence of SEQ ID NO: 89, a variant thereof or a fragment thereof. In one embodiment, the recombinant yeast host cell is modified for cytosolic enzymatic function and/or expression of these mitochondrial LDH of the DLD1 and/or CYB2 genes for the production of lactic acid. In another embodiment, the mitchondrial LDH enzyme is from a yeast, for example from the species Saccharomyces and in a further embodiment from Saccharomyces cerevisiae.

In some embodiments, the recombinant yeast host cell is genetically engineered to express a mutated malate dehydrogenase having LDH activity. Malate dehydrogenase is an enzyme having highly similar structure to lactate dehydrogenase. In such embodiment, the native gene encoding for the malate dehydrogenase can be mutated in the recombinant yeast host cell. Alternatively or in combination, an heterologous nucleic acid molecule coding for a mutated malate dehydrogenase (exhibiting LDH activity) can be introduced in the recombinant yeast host cell. As such, the recombinant yeast host cell can comprises an heterologous nucleic acid coding for a mutated malate dehydrogenase exhibiting LDH activity. In an embodiment, when the malate dehydrogenase is from Escherichia coli, it can be mutated at position 153 (to replace the arginine residue which another residue, such as, for example, a cysteine) to provide LDH activity (Wight and Viola, 2001).

In embodiments in which the recombinant yeast host cell is intended to produce valencene as the flavour compound, the recombinant yeast host cell of the present disclosure includes (and in an embodiment expresses) an heterologous nucleic acid molecule coding for one or more heterologous polypeptide for the production of valencene, such as, for example, a famesyl diphosphate synthase and/or a valencene synthase. Proteins having famesyl disphosphate synthase activity catalyze the production of famesyl disphosphate whereas proteins having valencene synthase activity catalyze the conversion of famesyl disphosphate into valencene. In one embodiment, the one or more polypeptide is or comprises a famesyl diphosphate synthase (FDPS), a variant thereof or a fragment thereof. The FDPS can be derived, for example, from a Arabidopsis sp. (including but not limited to Arabidopsis thaliana and having, for example, the amino acid sequence of SEQ ID NO: 12), a Glycyrrhiza sp. (including but not limited to Glycyrrhiza uralensis and having, for example, the amino acid sequence of SEQ ID NO: 13), a Capsella sp. (including, but not limited to Capsella rubella and having, for example, the amino acid sequence of SEQ ID NO: 14) or from a Lupinus sp. (including but not limited to Lupinus angustifolius and having, for example, the amino acid sequence of SEQ ID NO: 16). Alternatively or in combination, the one or more polypeptide is or comprises a valencene synthase, a variant thereof or a fragment thereof. The valencene synthase can be derived from a Citrus sp. (including, but not limited to a Citrus sinensis and having, for example, the amino acid sequence of SEQ ID NO: 17 or to a Citrus junos and having, for example, the amino acid sequence of SEQ ID NO: 18), a Vitis sp. (including, but not limited to Vitis vinifera and having, for example, the amino acid sequence of SEQ ID NO: 19), a Callitropsis sp. (including, but not limited to Callitropsis nootkatensis and having, for example, the amino acid sequence of SEQ ID NO: 20) or from a Populus sp. (including, but not limited to, Populus trichocarpa and having, for example, the amino acid sequence of SEQ ID NO: 21).

In embodiments in which the recombinant yeast host cell is intended to produce, nootkatone as the flavor compound, the recombinant yeast host cell of the present disclosure includes (and in an embodiment expresses) an heterologous nucleic acid molecule coding for one or more polypeptide for the production of nootkatone, such as, for example, a famesyl diphosphate synthase (FDPS), a valencene synthase, a cytochrome P450 oxygenase, a cytochrome P450 hydrozylase and/or a valencene oxidase. The nootkatone flavor can be produced by converting valencene into nootkatoone using a valencene oxidase (Cankar et al., 20014) or a combination of a cytochrome P450 oxygenase and a cytochrome P450 hydroxylase (Wriessnegger et al., 2014). In one embodiment, the one or more polypeptide is or comprises a famesyl diphosphate synthase (FDPS), a variant thereof or a fragment thereof. In one embodiment, the one or more polypeptide is or comprises a famesyl diphosphate synthase (FDPS), a variant thereof or a fragment thereof. The FDPS can be derived, for example, from a Arabidopsis sp. (including but not limited to Arabidopsis thaliana and having, for example, the amino acid sequence of SEQ ID NO: 12), a Glycyrrhiza sp. (including but not limited to Glycynhiza uralensis and having, for example, the amino acid sequence of SEQ ID NO: 13), a Capsella sp. (including, but not limited to Capsella rubella and having, for example, the amino acid sequence of SEQ ID NO: 14) or from a Lupinus sp. (including but not limited to Lupinus angustifolius and having, for example, the amino acid sequence of SEQ ID NO: 16). Alternatively or in combination, the one or more polypeptide comprises a valencene synthase, a variant thereof or a fragment thereof. The valencene synthase can be derived from a Citrus sp. (including, but not limited to a Citrus sinensis and having, for example, the amino acid sequence of SEQ ID NO: 17 or to a Citrus junos and having, for example, the amino acid sequence of SEQ ID NO: 18), a Vitis sp. (including, but not limited to Vitis vinifera and having, for example, the amino acid sequence of SEQ ID NO: 19), a Callitropsis sp. (including, but not limited to Callilropsis nootkatensis and having, for example, the amino acid sequence of SEQ ID NO: 20) or from a Populus sp. (including, but not limited to, Populus trichocarpa and having, for example, the amino acid sequence of SEQ ID NO: 21). Alternatively or in combination, the one or more polypeptide is or comprises a cytochrome P450 oxygenase. The cytochrome P450 oxygenase can be derived from a Bacillus sp. (including, but not limited to Bacillus subtilis and having, for example, the amino acid sequence of SEQ ID NO: 22); to a Bacillus amyloliquefaciens and having, for example, the amino acid sequence of SEQ ID NO: 23); to a Bacillus halotolerans and having for example, the amino acid sequence of SEQ ID NO: 24); to a Bacillus nakamurai and having, for example, the amino acid sequence of SEQ ID NO: 25) or to a Bacillus velezensis and having, for example, the amino acid sequence of SEQ ID NO: 26). Alternatively or in combination, the one or more polypeptide is or comprises a cytochrome P450 hydroxylase. The cytochrome P450 hydrozylase cane be derived from a Hyoscyamus sp. (including, but not limited to, Hyoscyamus muticus and having, for example, the amino acid sequence of SEQ ID NO: 27), a Nicotiana sp. (including, but not limited to Nicotiana attenuate and having, for example, the amino acid sequence of SEQ ID NO: 28), a Solanum sp. (including, but not limited to Solanum tuberosum and having, for example, the amino acid sequence of SEQ ID NO: 29; to Solanum pennellii and having, for example, the amino acid sequence of SEQ ID NO: 31) or from a Capsicum sp. (including, but not limited to Capsicum annuum and having, for example, the amino acid sequence of SEQ ID NO: 30). Alternatively or in combination, the one or more polypeptide is or comprises a cytochrome P450 reducatase. The cytochrome P450 reductase can be derived from Arabidopsis sp. (including, but not limited to, Arabidopsis thaliana and having, for example, the amino acid sequence of SEQ ID NO: 32), Brassica sp. (including, but not limited to, Brassica napus and having, for example, the amino acid sequence of SEQ ID NO: 33), Tarenaya sp. (including, but not limited to Tarenaya hassleriana and having, for example, the amino acid sequence of SEQ ID NO: 34), Quercus sp. (including, but not limited to Quercus suber and having, for example, the amino acid sequence of SEQ ID NO: 35) or from Prunus sp. (including, but not limited to Prunus persica and having, for example, the amino acid sequence of SEQ ID NO: 36). Alternatively or in combination, the one or more polypeptide is or comprises a valencene oxidase. The valencene oxidase can be derived from Callitopsis sp. (including, but not limited to, Callitropsis nootkatensis and having, for example, the amino acid sequence of SEQ ID NO: 37).

In some embodiments, when the recombinant yeast host cell is intended to produce valencene or nootkaone, it may be advantageous to provide a yeast host cell expressing a polypeptide having the 3-hydroxy-3-methylglutaryl-coenzyme A reductase 1 (HMG1) activity or further modify the cell so as to increase the activity of HMG1. This can be done for example, by including one or more copies of the gene encoding HMG1 or a corresponding gene ortholog in the yeast genome. In the context of the present disclosure, an “HMG1 gene ortholog” is understood to be a gene in a different species that evolved from a common ancestral gene by speciation. Genes encoding HMG1 or corresponding orthologs include, but are not limited to, proteins having the GenBank Accession number CAA86503.1 and KZV08767.1 (S. cerevisiae), CAA70691.1 (A. thaliana) and XP_566774.1 (Cryptococcus neoformans var. neoformans JEC21).

In an embodiment in which the recombinant yeast host cell is intended to produce vanillin as the flavour compound. In order to produce the vanillin as the flavor compound, it is possible to modify the recombinant yeast host cell of the present disclosure to include (and in an embodiment to express) an heterologous nucleic acid molecule coding for a feruloyl-CoA synthetase (FCS) and/or an enoyl-coA hydratase (ECH, also known as feruloyl-CoA hydratase or FCH). Alternatively, it is possible to modify the recombinant yeast host cell to produce directly vanillin from ferulic acid to include (and in an embodiment to express), a vanilin synthase. In one embodiment, the one or more polypeptide is or comprises a feruloyl-CoA synthetase (FCS). The feruloyl-coA synthetase can be derived from a Pseudomonas sp. (including, but not limited to, Pseudomonas fluorescens and having, for example, the amino acid sequence of SEQ ID NO: 38; Pseudomonas syringae and having, for example, the amino acid sequence of SEQ ID NO: 41), a Streptomyces sp. (including, but not limited to a Streptomyces sp. V-1 and having, for example, the amino acid sequence of SEQ ID NO: 39), a Sphingomonas sp. (including, but not limited to Sphingomonas paucimobilis and having, for example, the amino acid sequence of SEQ ID NO: 40) or from Nocardia sp. (including, but not limited to, Nocardia amikacinitolerans and having, for example, the amino acid sequence of SEQ ID NO: 42). Alternatively or in combination, the one or more polypeptide is or comprises an enoyl-CoA hydratase (ECH). The enoyl-CoA hydratase can be derived from a Pseudomonas sp. (including, but not limited to, Pseudomonas fluorescens and having, for example, the amino acid sequence of SEQ ID NO: 43; Pseudomonas syringae and having, for example, the amino acid sequence of SEQ ID NO: 46), a Streptomyces sp. (including, but not limited to a Streptomyces sp. V-1 and having, for example, the amino acid sequence of SEQ ID NO: 44), a Sphingomonas sp. (including, but not limited to Sphingomonas paucimobilis and having, for example, the amino acid sequence of SEQ ID NO: 45) or from Saocharopolyspora sp. (including, but not limited to, Saccharopolyspora flava and having, for example, the amino acid sequence of SEQ ID NO: 47). Alternatively or in combination, the one or more polypeptide is or comprises a vanillin synthase. The vanillin synthase can be derived from a Vanilla sp. (including, but not limited to, Vanilla planifolia and having, for example, the amino acid sequence of SEQ ID NO: 48) or from Glechoma sp. (including, but not limited to, Glechoma hederacea and having, for example, the amino acid sequence of SEQ ID NO: 49).

In some embodiments, the recombinant yeast host cell making the vanillin flavour compound is genetically engineered so as to no longer have phenylacrylic acid decarboxylase (PAD1) enzymatic activity. For example, the recombinant yeast host cell can be modified to remove in total or in part the PAD1 gene and/or its corresponding ortholog. In the context of the present disclosure, an “PAD1 gene ortholog” is understood to be a gene in a different species that evolved from a common ancestral gene by speciation. In the context of the present disclosure, a PAD1 ortholog retains the same function, e.g. it exhibits phenylacrylic acid decarboxylase enzymatic activity. This reduction or inhibition in PAD1 activity can be achieved by disrupting the open reading frame of the gene encoding PAD1 or its corresponding ortholog. This can be achieved by removing and/or adding one or more nucleic acid residues in the open reading frame of the PAD1 gene or gene ortholog. In an embodiment, the PAD1 gene can be disrupted by adding the heterologous nucleic acid molecule encoding for the one or more polypeptides for making the vanillin compound.

In an embodiment in which the recombinant yeast host cell is intended to produce isoamyl acetate as the flavour compound, the recombinant yeast host cell of the present disclosure includes (and in an embodiment expresses) an heterologous nucleic acid molecule coding one or more heterologous polypeptide for the production of isoamyl acetate, such as, for example, an alcohol acetyl transferase, a variant thereof or a fragment thereof. The alcohol acetyl transferase may comprise ATF1 and/or ATF2 alcohol acetyl transferase. In one embodiment, the one or more polypeptide is or comprises a ATF1 alcohol acetyl transferase. The alcohol acetyl transferase ATF1 can be derived, for example, from a Saccharomyces sp. (including but not limited to, Saccharomyces cerevisiae and having, for example, the amino acid sequence of SEQ ID NO: 51; to Saccharomyces pastorianus and having, for example, the amino acid sequence of SEQ ID NO: 50; to Saccharomyces kudriavzevii and having, for example, the amino acid sequence of SEQ ID NO: 52). In one embodiment, the one or more polypeptide is or comprises an ATF2 alcohol acetyl transferase. The alcohol acetyl transferase ATF2 can be derived, for example, from a Saccharomyces sp. (including but not limited to, Saccharomyces cerevisiae and having, for example, the amino acid sequence of SEQ ID NO: 53; to Saccharomyces eubayanus and having, for example, the amino acid sequence of SEQ ID NO: 54).

In embodiments in which the recombinant yeast host cell is intended to produce isoamyl acetate as the flavour compound, it may be advantageous to provide a yeast host cell expressing a native ATF enzyme or further modify the recombinant yeast host cell to overexpress an ATF enzyme. for example by cloning a promoter for overexpressing for controlling the expression of the native ATF enzyme. In another embodiment, the recombinant yeast host cell can be selected to express a native ATF enzyme (in addition to the heterologous ATF enzyme). This can be done for example, by including one or more copies of the gene encoding ATF enzyme or a corresponding gene ortholog in the yeast genome. In the context of the present disclosure, an “ATF gene ortholog” is understood to be a gene in a different species that evolved from a common ancestral gene by speciation.

In an embodiment in which the recombinant yeast host cell is intended to produce 4-(4-hydroxyphenyl)-2-butanone as the flavour compound, the recombinant yeast host cell of the present disclosure includes (and in an embodiment expresses) an heterologous nucleic acid molecule coding one or more heterologous polypeptide for the production of coumaric acid from phenylalanine as well as 4-(4-hydroxyphenyl)-2-butanone from coumaric acid. Heterologous polypeptides capable of converting phenylalanine into coumeric acid include, without limitation, phenylalanine-ammonium lyase (PAL) and/or cinnamate-4-hydroxylase (C4L). Heterologous polypeptides capable of converting coumeric acid into 4-(4-hydroxyphenyl)-2-butanone include, without limitation, coumarate-CoA ligase (4CL) and/or a benzalacetone synthase (BAS). In one embodiment, the one or more heterologous polypeptides is or comprises a phenylalanine-ammonium lyase (PAL), a variant thereof or a fragment thereof. In some embodiments, the PAL is derived from Rhodosporidium sp. (including, but not limited to Rhodosporidium toruloides and having, for example, the amino acid sequence of SEQ ID NO: 78). In one embodiment, the one or more heterologous polypeptides is or comprises a C4L, a variant thereof or a fragment thereof. For example, the C4L can be derived from Arabidopsis sp. (including, but not limited to Arabidopsis thaliana and having, for example, the amino acid sequence of SEQ ID NO: 80). In an embodiment, the one or more heterologous polypeptide is or comprises a coumarate-CoA ligase (4CL), a variant thereof or a fragment thereof. In another embodiment, 4CL is derived from Petroselinum sp. (including but not limited to Petroselinum crispum and having, for example, the amino acid sequence of SEQ ID NO: 56 or 83), Arabidopsis sp. (including, but not limited to Arabidopsis thaliana and having, for example, the amino acid sequence of SEQ ID NO: 55 or 84), a Paulownia sp. (including, but not limited to Paulownia fortune and having, for example, the amino acid sequence of SEQ ID NO: 57), Brassica sp. (including, but not limited to Brassica napus and having, for example, the amino acid sequence of SEQ ID NO: 58) or from Capsicum sp. (including, but not limited to Capsicum baccatum and having, for example, the amino acid sequence of SEQ ID NOL 59). In another embodiment, the one or more heterologous polypeptide is or comprises a benzalacetone synthase (BAS), a variant thereof or a fragment thereof. In another embodiment, BAS is derived from Rheum sp. (including but not limited to Rheum palmatum and having, for example, the amino acid sequence of SEQ ID NO: 60 or 61), Polygonum sp. (including, but not limited to Polygonum cuspidatum and having, for example, the amino acid sequence of SEQ ID NO; 62), Camellia sp. (including, but not limited to Camellia sinensis and having, for example, the amino acid sequence of SEQ ID NO: 63) or from Vitis sp. (including, but not limited to Vitis vinifera and having, for example, the amino acid sequence of SEQ ID NO: 64).

In some embodiments, the one or more heterologous protein is or comprises a chimeric polypeptide having 4CL and BAS activity. In such embodiment, a polypeptide having 4CL activity can be fused to a polypeptide having BAS activity either directly or via the use of an amino acid linker (for example, the amino acid linker having the amino acid sequence of SEQ ID NO: 85). In one embodiment, the carboxyl terminus of the polypeptide having 4CL activity can be linked (directly or indirectly via the use of an amino acid linker) to the amino terminus of the polypeptide having BAS activity. In such embodiment, the chimeric polypeptide can have the amino acid sequence of SEQ ID NO: 81 or 82. In another embodiment of the chimeric polypeptide, the carboxyl terminus of the polypeptide having BAS activity can be linked (directly or indirectly via the use of an amino acid linker) to the amino terminus of the polypeptide having 4CL activity.

In embodiments in which the recombinant yeast host cell is intended to produce 4-(4-hydroxyphenyl)-2-butanone as the flavour compound, it may be advantageous to provide a yeast host cell expressing a native benzylacetone reductase enzyme or further modify the recombinant yeast host cell to overexpress a benzylacetone reductase enzyme. for example by cloning a promoter for overexpressing for controlling the expression of the native benzylacetone reductase enzyme. In another embodiment, the recombinant yeast host cell can be selected to express a native benzylacetone reductase enzyme (in addition to the heterologous ATF enzyme). This can be done for example, by including one or more copies of the gene encoding the benzylacetone reductase enzyme or a corresponding gene ortholog in the yeast genome. In the context of the present disclosure, a “benzylacetone reductase gene ortholog” is understood to be a gene in a different species that evolved from a common ancestral gene by speciation.

In an embodiment in which the recombinant yeast host cell is intended to produce 4-ethyl-phenol and/or 4-ethyl guiacol as the flavour compound, the recombinant yeast host cell of the present disclosure includes (and in an embodiment expresses) an heterologous nucleic acid molecule coding one or more heterologous polypeptide for the production of 4-ethyl-phenol and/or 4-ethyl guiacol, such as, for example, a vinylphenol reductase, a variant thereof or a fragment thereof. In an embodiment, the vinylphenol reductase is derived from Brettanomyces sp. (including, but not limited to, Brettanomyces bruxellensis and having, for example, the amino acid sequence of SEQ ID NO: 65, 66 or 67) or from Ogataea sp. (including, but not limited to Ogataea parapolymorpha and having, for example, the amino acid sequence of SEQ ID NO: 68).

In an embodiment in which the recombinant yeast host cell is intended to produce phenylethyl alcohol as the flavour compound, the recombinant yeast host cell of the present disclosure includes (and in an embodiment expresses) an heterologous nucleic acid molecule coding one or more heterologous polypeptide for the production of phenylethyl alcohol, such as, for example, ARO8, ARO9, PDC1, PDC5, PDC6, ARO10, SFA1, ADH4, and/or ADH5. In an embodiment, the one or more heterologous polypeptide is or comprises ARO8 (having, for example, an amino acid sequence of SEQ ID NO: 91), a variant thereof or a fragment thereof. In an embodiment, the one or more heterologous polypeptide is or comprises ARO9 (having for example the amino acid sequence of SEQ ID NO: 92), a variant thereof or a fragment thereof. In an embodiment, the one or more heterologous polypeptide is or comprises PCD1 (having, for example, the amino acid sequence of SEQ ID NO: 93), a variant thereof or a fragment thereof. In an embodiment, the one or more heterologous polypeptide is or comprises PDC5 (having, for example, the amino acid sequence of SEQ ID NO: 94), a variant thereof or a fragment thereof. In an embodiment, the one or more heterologous polypeptide is or comprises PDC6 (having, for example, the amino acid sequence of SEQ ID NO: 95), a variant thereof or a fragment thereof. In an embodiment, the one or more heterologous polypeptide is or comprises ARO10 (having, for example, the amino acid sequence of SEQ ID NO: 96), a variant thereof or a fragment thereof. In an embodiment, the one or more heterologous polypeptide is or comprises SFA1 (having, for example, the amino acid sequence of SEQ ID NO: 97), a variant thereof or a fragment thereof. In an embodiment, the one or more heterologous polypeptide is or comprises ADH4 (having, for example, the amino acid sequence of SEQ ID NO: 98), a variant thereof or a fragment thereof. In an embodiment, the one or more heterologous polypeptide is or comprises ADH5 (having, for example, the amino acid sequence of SEQ ID NO: 99), a variant thereof or a fragment thereof.

In embodiments in which the recombinant yeast host cell is intended to produce phenylethyl alcohol as the flavour compound, it may be advantageous to provide a yeast host cell expressing at least one of native ARO8, ARO9, ARO10, PDC1, PDC5, PDC6, SFA1, ADH4 or ADH5 or further modify the recombinant yeast host cell to overexpress at least one at least one of ARO8, ARO9, ARO10, PDC1, PDC5, PDC6, SFA1, ADH4 or ADH5. for example by cloning a promoter for overexpressing for controlling the expression of the native benzylacetone reductase enzyme. In another embodiment, the recombinant yeast host cell can be selected to express a native ARO8, ARO9, ARO10, PDC1, PDC5, PDC6, SFA1, ADH4 and/or ADH5 (in addition to the heterologous at least one of ARO8, ARO9, ARO10, PDC1, PDC5, PDC6, SFA1, ADH4 and/or ADH5). This can be done for example, by including one or more copies of the gene encoding at least one at least one of ARO8, ARO9, ARO10, PDC1, PDC5, PDC6, SFA1, ADH4 or ADH5 or a corresponding gene ortholog in the yeast genome. In the context of the present disclosure, a “gene ortholog” is understood to be a gene in a different species that evolved from a common ancestral gene by speciation.

In an embodiment in which the recombinant yeast host cell is intended to produce ethyl capraote as the flavour compound, the recombinant yeast host cell of the present disclosure includes (and in an embodiment expresses) an heterologous nucleic acid molecule coding one or more heterologous polypeptide for the production of ethyl capraote, such as, for example, FAS2, a variant thereof, a mutant thereof, or a fragment thereof. In an embodiment, the FAS2 enzyme has the amino acid sequence of SEQ ID NO: 86, is a variant of the amino acid sequence of SEQ ID NO: 86 or is a fragment of the amino acid sequence of SEQ ID NO: 86. In an embodiment, the mutated FAS2 enzyme has the amino acid sequence of SEQ ID NO: 87 or 88, is a variant of the amino acid sequence of SEQ ID NO: 87 or 88 or is a fragment of the amino acid sequence of SEQ ID NO: 87 or 88.

In an embodiment in which the recombinant yeast host cell is intended to produce vanillyloctanamide as the flavour compound, the recombinant yeast host cell of the present disclosure includes (and in an embodiment expresses) an heterologous nucleic acid molecule coding one or more heterologous polypeptide for the production of vanillyloctanamide, such as, for example, capsaicin synthase and/or pAMT1. In an embodiment, the one or more heterologous polypeptide is or comprises capsaicin synthase, a variant thereof or a fragment thereof. In an embodiment, the capsaicin synthase (or acyltransferase) is derived from Capsicum sp. (including, but not limited to C. annuum acylsugar and having, for example, amino acid sequence of SEQ ID NO: 69 or 73). In an embodiment, the capsaicin synthase (or acyltransferase) is derived from Capsicum sp. (including, but not limited to C. frutescense and having, for example, amino acid sequence of SEQ ID NO: 70). In an embodiment, the capsaicin synthase (or acyltransferase) is derived from Solanum sp. (including, but not limited to S. lycospersicum and having, for example, amino acid sequence of SEQ ID NO: 71). In an embodiment, the capsaicin synthase (or acyltransferase) is derived from Capsicum sp. (including, but not limited to C. chacoense and having, for example, amino acid sequence of SEQ ID NO: 72). In an embodiment, the pAMT is derived from Capsicum sp. (including, but not limited to C. chinesne and having, for example, amino acid sequence of SEQ ID NO: 74 or 76). In an embodiment, the pAMT is derived from Capsicum sp. (including, but not limited to C. frutescense and having, for example, amino acid sequence of SEQ ID NO: 75). In an embodiment, the pAMT is derived from Capsicum sp. (including, but not limited to C. baccatum and having, for example, amino acid sequence of SEQ ID NO: 77). In an embodiment, the pAMT is derived from Solanum sp. (including, but not limited to S. lycospersicum and having, for example, amino acid sequence of SEQ ID NO: 78).

The heterologous polypeptide encoded by the heterologous nucleic acid molecule (either for the production of ethanol and/or for the production of the flavour compound) can be a variant of a known/native polypeptide. A variant comprises at least one amino acid difference when compared to the amino acid sequence of the native polypeptide. As used herein, a variant refers to alterations in the amino acid sequence that do not adversely affect the biological functions of the polypeptide. A substitution, insertion or deletion is said to adversely affect the polypeptide when the altered sequence prevents or disrupts a biological function associated with the polypeptide. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the polypeptide. The polypeptide variants have at least 30%, 40%, 50%, 55%, 60%, 65%, 70%, 75%, 80%, 85%, 90%, 95%, 96%, 97%, 98% or 99% identity to the polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.).

The variant heterologous polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the polypeptide can be a conservative variant or an allelic variant.

The heterologous polypeptide encoded by the heterologous nucleic acid molecule (either for the production of ethanol and/or for the production of the flavour compound) can be a fragment of a known/native polypeptide. Polypeptide “fragments” have at least at least 100, 200, 300, 400, or more consecutive amino acids of the polypeptide. A fragment comprises at least one less amino acid residue when compared to the amino acid sequence of the known/native polypeptide and still possess the enzymatic activity of the full-length polypeptide. In some embodiments, fragments of the polypeptide can be employed for producing the corresponding full-length polypeptide by peptide synthesis. Therefore, the fragments can be employed as intermediates for producing the full-length proteins.

The recombinant host cell can be provided as a fermenting agent for making a flavoured alcoholic beverage. In such embodiment, the fermenting agent can include, without limitation a nutrient for the fermenting agent (for example, a carbon source).

The recombinant host cell can be provided in combination with another fermenting and non-genetically-modified organism (such as, for example, a non-genetically-modified yeast). The can be useful to reach, but not surpass, the maximal amount of the flavour compound in the resulting flavoured alcoholic beverage. In an embodiment, the percentage (in cell weight) of the recombinant yeast host cell in the combination can be at least 10, 20, 30, 40, 50, 60, 70, 80, 90% or more. Alternatively or in combination, the percentage (in cell weight) of the non-genetically-modified yeast in the combination can be no more than 90, 80, 70, 60, 50, 40, 30, 20, 10% or less. In an embodiment, the percentage (in cell weight) of the recombinant yeast host cell in the combination can be no more than 90, 80, 70, 60, 50, 40, 30, 20, 10% or less. Alternatively of in combination, the percentage (in cell weight) of the non-genetically-modified yeast in the combination can be at least 10, 20, 30, 40, 50, 60, 70, 80, 90% or more. In such embodiment, the combination can include, without limitation a nutrient for the combination (for example, a carbon source).

### Tools for Making the Recombinant Yeast Host Cell

In order to make the recombinant yeast host cells, heterologous nucleic acid molecules (also referred to as expression cassettes) are made in vitro and introduced into the recombinant yeast host cell in order to allow the recombinant expression of the heterologous polypeptide.

The heterologous nucleic acid molecules of the present disclosure comprise a coding region for the heterologous polypeptide. A DNA or RNA “coding region” is a DNA or RNA molecule (preferably a DNA molecule) which is transcribed and/or translated into a heterologous polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell (such as the recombinant yeast host cell of the present disclosure), a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a recombinant host cell. In eukaryotic cells, polyadenylation signals are considered control regions.

In some embodiments, the heterologous nucleic acid molecules of the present disclosure include a promoter as well as a coding sequence for a heterologous polypeptide. The heterologous nucleic acid sequence can also include a terminator. In the heterologous nucleic acid molecules of the present disclosure, the promoter and the terminator (when present) are operatively linked to the nucleic acid coding sequence of the heterologous polypeptide, e.g., they control the expression and the termination of expression of the nucleic acid sequence of the heterologous polypeptide. The heterologous nucleic acid molecules of the present disclosure can also include a nucleic acid coding for a signal peptide, e.g., a short peptide sequence for exporting the heterologous polypeptide outside the host cell. When present, the nucleic acid sequence coding for the signal peptide is directly located upstream and in frame of the nucleic acid sequence coding for the heterologous polypeptide.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous polypeptide from the nucleic acid molecule. In an embodiment, the promoter can be located upstream (5) of the nucleic acid sequence coding for the heterologous protein. In still another embodiment, the promoter can be located downstream (3) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promotor or a combination of different promoters.

In the context of the present disclosure, the promoter controlling the expression of the heterologous polypeptide can be a constitutive promoter (such as, for example, tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), hxk1 (e.g., the promoter of the hxk1 gene) and pgk1p (e.g., the promoter of the pgk1 gene). In some embodiment, the promoter is adh1p (e.g., the promoter of the adh1 gene). However, is some embodiments, it is preferable to limit the expression of the polypeptide. As such, the promoter controlling the expression of the heterologous polypeptide can be an inducible or modulated promoters such as, for example, a glucose-regulated promoter (e.g., the promoter of the hxt7 gene (referred to as hxt7p)) or a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In an embodiment, the promoter is an anaerobic-regulated promoters, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gdp1 gene), cdc19p (e.g., the promoter of the cdc19 gene), eno2p (e.g., the promoter of the eno2 gene), pdc1p (e.g., the promoter of the pdc1 gene), hxt3p (e.g., the promoter of the hxt3 gene), dan1 (e.g., the promoter of the dan1 gene) and tpi1p (e.g., the promoter of the tpi1 gene). In an embodiment, the promoter used to allow the expression of the heterologous polypeptide is the adh1p. One or more promoters can be used to allow the expression of each heterologous polypeptides in the recombinant yeast host cell.

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide. Usually, functional fragments are either 5′ and/or 3′ truncation of one or more nucleic acid residue from the native promoter nucleic acid sequence.

In some embodiments, the nucleic acid molecules include a one or a combination of terminator sequence(s) to end the translation of the heterologous polypeptide. The terminator can be native or heterologous to the nucleic acid sequence encoding the heterologous polypeptide. In some embodiments, one or more terminators can be used. In some embodiments, the terminator comprises the terminator derived from is from the dit1 gene, from the idp1 gene, from the gpm1 gene, from the pma1 gene, from the tdh3 gene, from the hxt2 gene, from the adh3 gene, from the cyc1 gene, from the pgk1 gene and/or from the ira2 gene. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein. In the context of the present disclosure, the expression “functional fragment of a terminator” refers to a shorter nucleic acid sequence than the native terminator which retain the ability to end the expression of the nucleic acid sequence coding for the heterologous protein.

The heterologous nucleic acid molecule encoding the one or more heterologous polypeptide, variant or fragment thereof can be integrated in the genome of the yeast host cell. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

The present disclosure also provides nucleic acid molecules for modifying the yeast host cell so as to allow the expression of the one or more heterologous polypeptide, variants or fragments thereof. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not after the properties or activities of the encoded polypeptide, variants or fragments.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The present disclosure also provides heterologous nucleic acid molecules that are hybridizable to the complement nucleic acid molecules encoding the heterologous polypeptides as well as variants or fragments. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acid molecules contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order RNA:RNA, DNA:RNA, DNA:DNA. For hybrids of greater than 100 nucleotides in length, equations for calculating Tm have been derived. For hybridizations with shorter nucleic acids, i.e., oligonucleotides, the position of mismatches becomes more important, and the length of the oligonucleotide determines its specificity. In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

### Processes for Making Alcoholic Beverage Products

The recombinant yeast host cell of the present disclosure have been designed to be used in the preparation of flavoured and alcoholic beverage products for human consumption. The present disclosure thus provides a process comprising contacting the recombinant yeast host cell of the present disclosure with a carbohydrate to provide a mixture and fermenting the mixture so as to obtain at most 3% v/w of the flavour compound and at least 5 g/L of ethanol once the carbohydrates have been converted. The fermentation can be conducted in the presence of or by the recombinant yeast host cell described herein. In some embodiments, it may be advantageous to provide the recombinant yeast host cell of the present disclosure as a fermentation agent. In one embodiment, a fermenting agent for making a flavoured and fermented alcoholic beverage comprising or consisting essentially of the recombinant yeast host cell described herein. As used herein, “consisting essentially of” in reference to a fermenting agent refers to a population of fermenting organisms which do not include a substantial amount of additional fermenting or flavoring organisms which participate to the fermentation process. In an embodiment, a fermenting agent consisting essentially of the recombinant yeast host cell of the present disclosure is made up of at least 80%, 85%, 90%, 95%, 99%, or 99.9% of the recombinant yeast host cell described herein. In still another embodiment, a fermenting agent consisting essentially of the recombinant yeast host cell of the present disclosure is a monoculture of one strain of a recombinant yeast host cell. Alternatively, a fermenting agent consisting essentially of the recombinant yeast host cell of the present disclosure is a combination of more than one strains of the recombinant yeast host cell described herein.

In a specific embodiment, the recombinant host cell can be provided in combination with another fermenting and non-genetically-modified organism (such as, for example, a non-genetically-modified yeast). The can be useful to reach, but not surpass, the maximal amount of the flavour compound in the resulting flavoured alcoholic beverage. In an embodiment, the percentage (in cell weight) of the recombinant yeast host cell in the combination can be at least 10, 20, 30, 40, 50, 60, 70, 80, 90% or more. Alternatively or in combination, the percentage (in cell weight) of the non-genetically-modified yeast in the combination can be no more than 90, 80, 70, 60, 50, 40, 30, 20, 10% or less. In an embodiment, the percentage (in cell weight) of the recombinant yeast host cell in the combination can be no more than 90, 80, 70, 60, 50, 40, 30, 20, 10% or less. Alternatively of in combination, the percentage (in cell weight) of the non-genetically-modified yeast in the combination can be at least 10, 20, 30, 40, 50, 60, 70, 80, 90% or more. In such embodiment, the combination can include, without limitation a nutrient for the combination (for example, a carbon source).

In an embodiment, the recombinant yeast host cell of the present disclosure can be used in a brewing process to make a beer (such as, for example, an ale or a lager beer). Process for making beer include, without limitation, contacting the recombinant yeast host cell (alone or in a combination) of the present disclosure with a wort as a carbohydrate substrate to provide maltose and maltotriose and ferment the wort until at least 5 g/L of ethanol is obtained and the flavor compound is produced once the carbohydrates have been converted. When the recombinant yeast host cell of the present disclosure is intended to produce lactic acid, the beer can be a sour beer (such as, for example, a sour ale or a sour lager beer). The process for making a beer can also include a step of making a wort, a step of secondary fermentation, a conditioning step, a filtering step and/or a sterilizing step. In some embodiments, the present disclosures excludes using recombinant yeast host cell intended to produce the 4-(4-hydroxyphenyl)-2-butanone compound flavor in a beer-making process.

Brewing typically contains four main ingredients: water, cereal, hops, and yeast. The brewing process begins with milling the partially germinated and dried grains (referred to as malted cereals), with the most common grain being barley. The malt is cracked during the milling process to break up the grain kernels and expose the starch molecules. The milled grains are transferred to a mash tun where it is mixed with warm water, typically between 37-76° C., activating the natural amylolytic enzymes (for example 60 to 69′C for amylases and/or 38 to 49° C. for proteases) which begin to degrade the starches creating fermentable sugars, primarily maltose and maltotriose. Optionally, exogenous enzyme can be added to further enhance sugar conversion and reduce viscosity. After approximately one to two hours, the mash is pumped to the lauter tun where the sugar water (now referred to as a wort), is separated from the spent grain. First, a mashout is typically performed in which the mash temperature is raised to >77° C. to inactivate the enzymes and preserve the sugar profile. The wort is then drained from the bottom as the lauter tun typically has a perforated or false bottom which allows the wort to filter through, leaving behind the solids. The wort is initially recirculated to the top of the lauter tun to allow the grains to compress and act as a natural filter. Once the wort begins to run clear with less grain particulates, the wort is transferred to the boil kettle. The grain bed is then sparged, the process of rinsing the grains with hot water to wash and extract as much of the sugar as possible.

The sparge is collected with the initial wort runoffs into the kettle which is boiled to both sterilize the wort, but also for hop additions to impart the aromatic and bitter qualities of the beer. The boiling can be performed to isomerize the hop's alpha acids, solubilizing them and enhancing the bitter taste. The earlier the hops are added in the boil, the more isomerization and bittering effect. Therefore, the hop schedule can be carefully designed for each recipe to balance the bitter and aromatic contributions of each hop species, with early boil additions targeting bittemess, mid-boil addition targeting both flavor and aroma, and late boil addition for aroma. Hops can also be added during the fermentation or maturation phases, a process called dry hopping, typically targeting extraction of essential oils from the hops which lend a strong aromatic profiles. Hops can also be added in the whirlpool, the end of the boiling phase in which the wort is stirred to create a vortex collecting all of the insoluble hop and grain residue at the bottom, enhancing the clarity of the wort. Some brewers whirlpool in the boil kettle itself, while others have a separate vessel. The wort is then passed through a heat exchanger to quickly cool the liquid as it is transferred to the fermenter. After a slight oxygenation step, the yeast is pitched and allowed to convert the sugars to alcohol and carbon dioxide. Typically for ales, Saccharomyces cerevisiae is pitched and incubated at 15 to 23° C. enhancing the ester and phenolic compound production of yeast, where lagers are pitched with Saccharomyces pastorianus and incubated at 10° C. or lower to reduce the yeast's flavor contribution. After primary fermentation, the beer can be transferred to a secondary fermentation, removing it from the spent yeast and allowing it to further condition and mature. Lagers typically can be stored for three to four weeks in cold storage to allow the remaining active yeast to consume the off flavor diacetyl. The conditioning can be used to add additional flavors (e.g., fruit, spice, or more hops). Some brewers will condition in bottles to also naturally carbonate the beer, while others can age in barrels or casks to further extract flavors and aromatics. After maturation, the beer can be filtered or pasteurized and transferred to the bright tank were it is carbonated, typically using forced carbonation with CO₂ tanks. The final beer product is then packaged in kegs, bottles, or cans.

In an embodiment, the recombinant yeast host cell of the present disclosure can be used in a distilling process. In such embodiment, the process includes contacting the recombinant yeast host cell (alone or in a combination) of the present disclosure with a carbohydrate source to create a mixture, fermenting the mixture and distilling the fermented mixture. In some embodiments, the present disclosures excludes using recombinant yeast host cell intended to produce the 4-(4-hydroxyphenyl)-2-butanone compound flavor in a distilled product-making process.

In some embodiments, the present disclosure excludes using the recombinant yeast host cell in a wine-making process. In some embodiments, the present disclosure excludes using recombinant yeast host cell intended to produce the 4-(4-hydroxyphenyl)-2-butanone compound flavor in a wine-making process.

In an embodiment, the alcoholic beverage products are flavoured alcoholic beverage products. Examples of alcoholic beverage products include, but are not limited to, beer (including sour beer), wine, cider, sparkling wine (including champagne), mead, brandy as well as brandy-based wine, whisky, rum, vodka, gin, tequila, mexcal, sake, or arrack. In an embodiment, the alcoholic beverage product excludes wines and champagnes. When used during the process for making flavoured alcoholic beverage products, the recombinant yeast host cells can be the sole fermenting organism that is added to the carbohydrate substrate. In other instances, the recombinant yeast host cells can be admixed with non-recombinant (e.g., wild-type) yeasts up to provide a combination for delivering the adequate dose of heterologous flavour producing activity. For example, the recombinant yeast host cell (which can be a recombinant Saccharomyces cerevisiae yeast host cell) can be combined in any ratio with a wild-type yeast host cell (which can be a wild-type non-recombinant Saccharomyces cerevisiae). In an embodiment, the ratio between recombinant:wild-type is between 1:1000 and 1000:1 and, in some additional embodiments, between 1:100 to 100:1.

In the process described herein, the recombinant yeast host cells of the present disclosure can be provided in an active form (e.g., liquid, compressed, or fluid-bed dried yeast), in a semi-active form (e.g., liquid, compressed, or fluid-bed dried), in an inactive form (e.g., drum- or spray-dried) as well as a mixture therefore. For example, the recombinant yeast host cells can be a combination of active and semi-active or inactive forms to provide the ratio and dose of the polypeptide required for making flavoured alcoholic beverage products. In an embodiment, the recombinant yeast host cells are provided in an active and dried form.

The present invention will be more readily understood by referring to the following examples which are given to illustrate the invention rather than to limit its scope.

### Example I—Heterologous Lactate Dehydrogenase Expression in an Ale Strain

Lactate dehydrogenase containing cassette was engineered into an ale brewing strain, M14629, with Rhizopus oryzae lactate dehydrogenase (SEQ ID NO: 2) at the FCY1 site under control of the constitutive ADH1 promoter (adh1p). Successfully transformed cells grew on YPD₄₀ agar plates containing 5-fluorocytosine. The resulting transformants were initially screened for lactic production by growing overnight in 5 mL of YPD and samples submitted for HPLC analysis (Table 4).

TABLE 4
Glucose, lactic acid, glycerol, and
ethanol levels of eight transformants after
overnight growth in YPD₄₀ media.

| | Glucose | Lactic Acid | Glycerol | Ethanol |
|---|---|---|---|---|
| Transformant # | (g/L) | (g/L) | (g/L) | (g/L) |
| 1 | 0.00 | 4.81 | 0.92 | 10.54 |
| 2 | 0.04 | 5.04 | 1.08 | 10.08 |
| 3 | 0.00 | 5.87 | 1.03 | 10.39 |
| 4 | 0.00 | 5.73 | 1.00 | 10.26 |
| 5 | 0.09 | 4.70 | 1.11 | 8.56 |
| 6 | 0.04 | 5.28 | 0.82 | 10.65 |
| 7 | 0.05 | 5.43 | 1.15 | 10.03 |
| 8 | 0.05 | 5.27 | 0.85 | 9.81 |

Strain M16141 (corresponding to transformant 6 in Table 4) was selected for further evaluations. More specifically, strain M16141 was further evaluated in a lab-scale wort fermentation. Both strain M16141 and the parent strain M14629 were grown overnight in 100 mL YP-maltose 80 g/L at room temperature and inoculated into 13° Plato dry malt extract with 0.01% isomerized hop oil at 175 mL volumes in 250 mL conical tubes in duplicates, and incubated at room temp (˜20° C.). The strains were dosed at 0.125 g dry cell weight (DCW), as well as in co-cultures with the M14629 and M16141 at either 50:50 (0.0625 g/L each) or 25:75 (0.03125 g/L for M14629 and 0.09375 g/L for M16141). Samples were collected every 24 h for HPLC analysis and specific gravity for a total of 144 h.

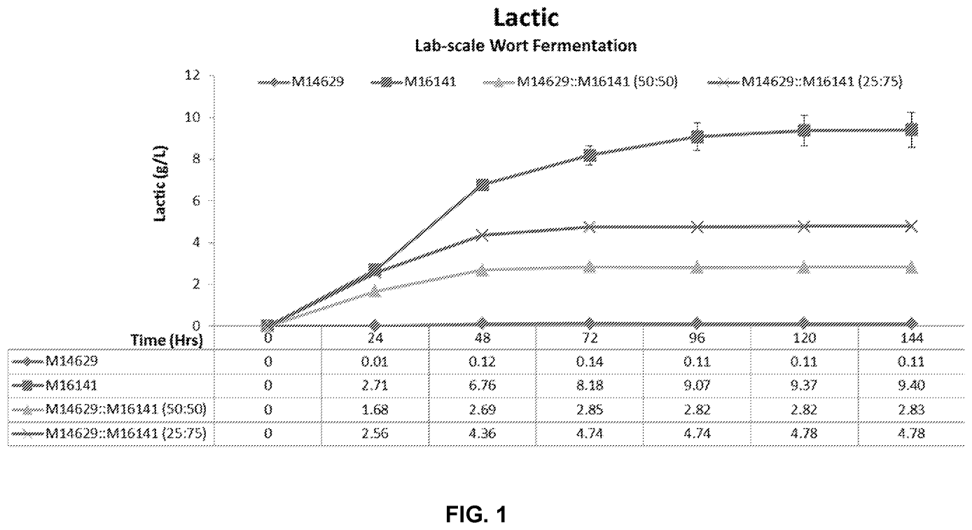

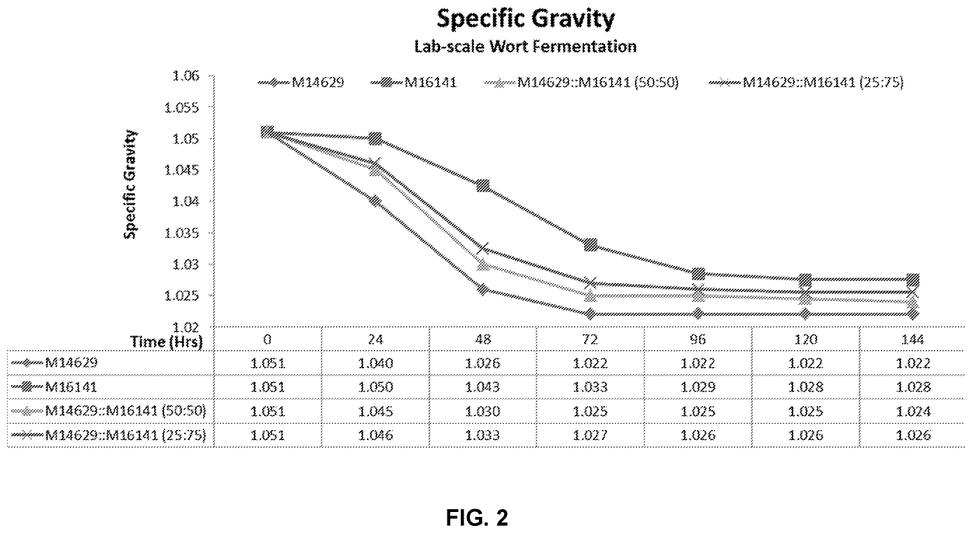

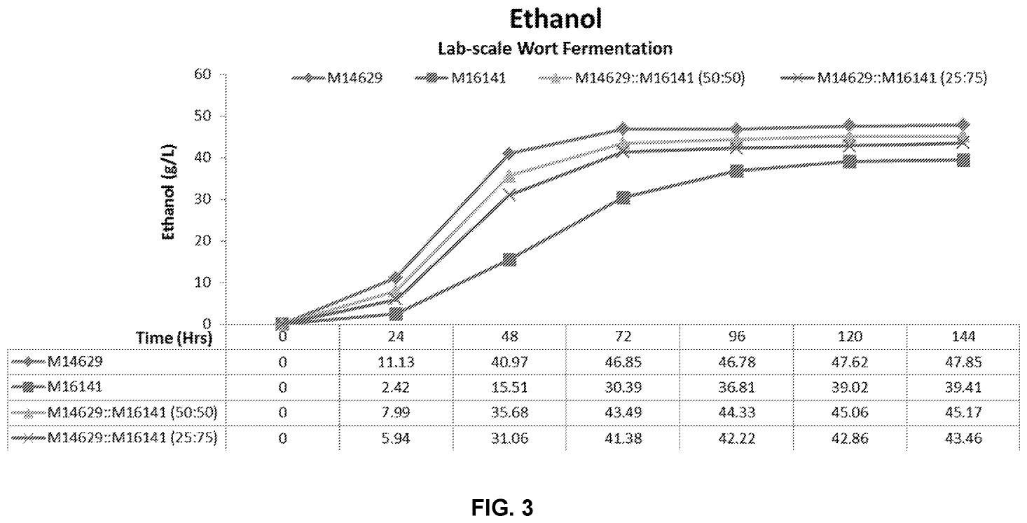

The recombinant LDH-producing strain, M16141, quickly produced lactic acid in the wort fermentation with 2.7 g/L after just 24 h and reaching 9 g/L within 96 h (FIG. 1). Strain M16141 produced 9.4 g/L at 144 h compared to a negligible 0.11 g/L for the parent strain, M14629. As expected, the co-cultures produced less lactic with the 50:50 culture ending with 2.8 g/L and the 25:75 at 4.78 g/L (both of which reached near final titers at 72 h). The performance of the co-cultures is shown in FIG. 2. The ethanol yield for strain M16141 was lower (when compared to the parent strain) as some of the carbon is shifted to lactic production rather than ethanol. The strain M16141 finished the fermentation with 39.4 g/L of ethanol which is lower compared to 47.9 g/L for strain M14629 (FIG. 3). The 50:50 and 25:75 co-cultures each finished the fermentation with 45.2 g/L and 43.5 g/L ethanol, respectively.

Given the results of the lab-scale ale fermentation, strain M16141 was then used as in a full scale beer fermentation. A 7.57 L (2.5 gallon) beer fermentation was performed using 454 g (1 lb) Pilsen dry malt extract and 2722 g (6 lbs) of Munich malt extract syrup and boiled for 60 mins. Hops were at the initial 60 min boil using German Perle at 3 alpha acid units (AAU), followed by Fuggle hops at 2 AAU and boiled for 50 mins and 1.3 AAU Fuggle hops for 30 min boil. The wort was then cooled to 75° C. and pitched at 0.125 g/L yeast that was propped in 80 g/L maltose media. The fermentation was incubated at room temperature with an airlock. Over the course of 256 h, strain M16141 produced 23.13 g/L lactic acid (Table 5).

TABLE 5
Glucose, lactic acid, glycerol, and
ethanol levels strains M14629 and
M16141 after 256 hours of growth in beer wort.

| | Lactic Acid | Glycerol | Ethanol | Total Sugars |
|---|---|---|---|---|
| Strain | (g/L) | (g/L) | (g/L) | (g/L) |
| M14629 | 0.96 | 2.46 | 38.18 | 10.49 |
| M16141 | 23.13 | 2.59 | 35.88 | 7.49 |

### Example II—Heterologous Lactate Dehydrogenase Expression in a Distilling Strain

Engineering of Lactate Dehydrogenase into Industrial Distilling Strain (M2390). Lactate dehydrogenase containing cassette was engineered into an industrial an distilling strain M2390. The cassette contained Rhizopus oryzae lactate dehydrogenase (SEQ ID NO: 1) at the IME1 site under control of the constitutive ADH1 promoter in transformation T4756. Successfully transformed cells grew on YPD₄₀ agar plates containing 5-fluoro-2′-deoxyuridine (FUDR).

The resulting transformants were initially screened for lactic production by growing for 24 hours in 25 mL of YPD₈₀ in capped and vented 50 mL minivials. Samples were submitted for HPLC analysis. Table 6 indicates provides the lactic acid productions for the strains and the transformants tested.

TABLE 6
Glucose, lactic acid, glycerol, and
ethanol levels of eight translormants after 24
hours of growth in minival YPD₈₀ media.

| Strains or | Glucose | Lactic | Glycerol | |
|---|---|---|---|---|
| Transformant | g/L | Acid g/L | g/L | Ethanolg/L |
| M2390 | 4.54 | 0.16 | 1.13 | 14.87 |
| 1 | 0.20 | 9.01 | 0.89 | 13.09 |
| 2 | 0.79 | 8.95 | 0.89 | 12.76 |
| 3 | 4.49 | 7.61 | 0.75 | 11.83 |
| 4 | 1.70 | 5.88 | 0.88 | 13.79 |
| 5 | 4.11 | 7.66 | 0.77 | 11.84 |
| 6 | 6.25 | 4.58 | 0.80 | 12.31 |
| 7 | 1.78 | 8.11 | 0.86 | 12.70 |
| 8 | 0.00 | 6.30 | 1.02 | 14.31 |

### Example III—Heterologous Isoamyl Acetate Expression in an Ale Strain

Isoamyl acetate containing cassette was engineered into an ale brewing strain, M14635, with Saccharomyces cerevisiae lactate dehydrogenase (ATF1) at the FCY1 site under control of the constitutive ADH1 promoter (adh1p) to generate strain M16626. Successfully transformed cells grew on YPD₄₀ agar plates containing 5-fluorocytosine.

Both strain M16626 and the parent strain M14635 were grown overnight in 100 mL YP-maltose 80 g/L at room temperature and inoculated into 13° Plato dry malt extract with 0.01% isomerized hop oil at 175 mL volumes in 250 mL conical tubes in duplicates, and incubated at room temp (˜20° C.). The strains were dosed at 0.125 g dry cell weight (DCW), as well as in co-cultures with the M14629 and M16141 at either 50:50 (0.0625 g/L each) or 25:75 (0.03125 g/L for M14629 and 0.09375 g/L for M16141). Samples were collected every 24 h for HPLC analysis and specific gravity for a total of 144 h.

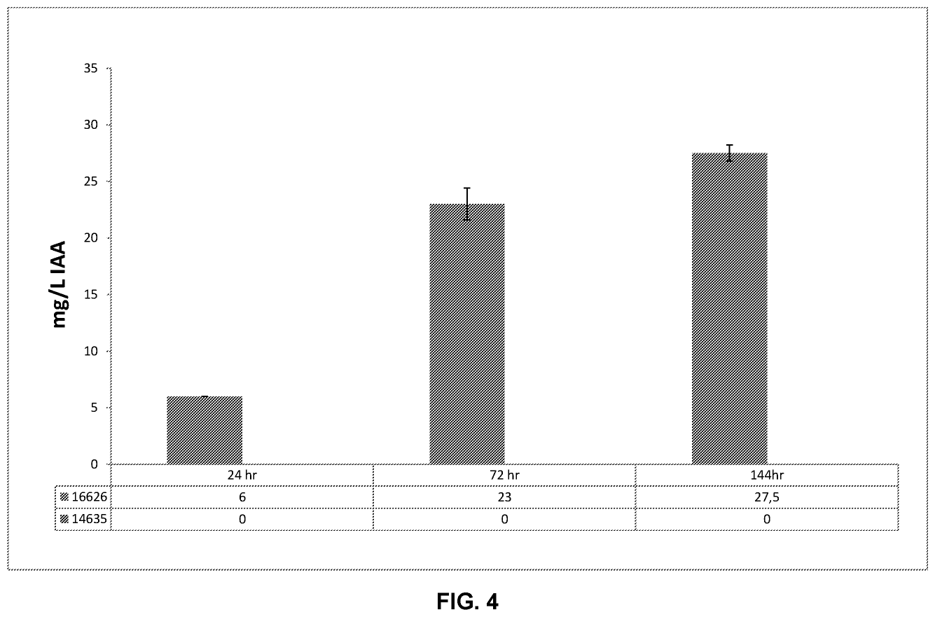

As shown on FIG. 4, strain M16626 produced 27.5 mg/L of isoamyl acetate after 144 h of fermentation, whereas no isoamyl acetate was produced in the parent strain.

### Example IV—Heterologous Lactacte Dehydrogenase Expression in an Ale Strain

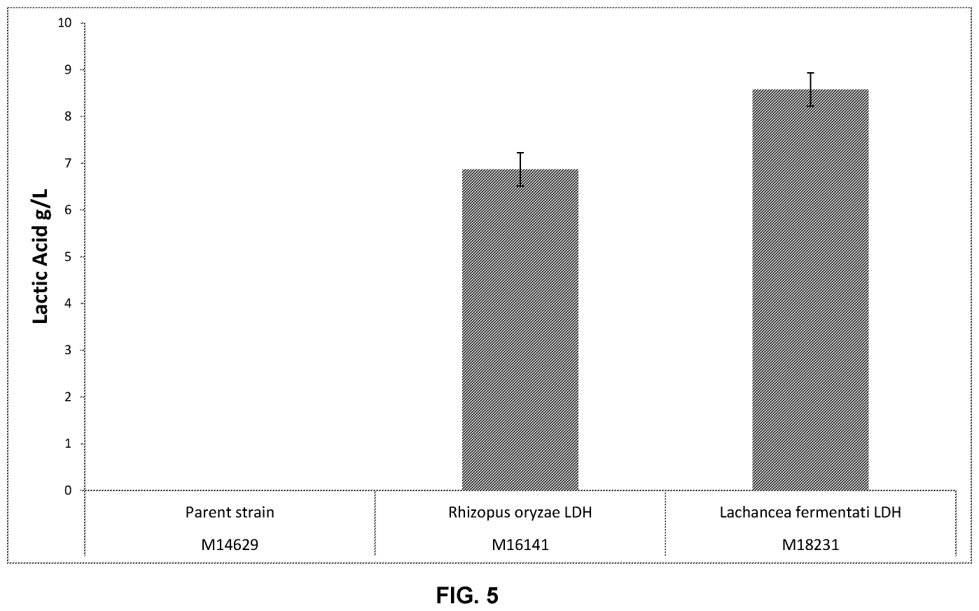

A lactate dehydrogenase from Lachancea fennentati (SEQ ID NO: 3) cassette was engineered into an ale brewing strain, M14629, at the FCY1 site under control of the constitutive ADH1 promoter (adh1p) to generate strain M18231. Successfully transformed cells were grown on YPD₄₀ agar plates containing 5-fluorocytosine. The resulting transformants were initially screened for lactic production by growing overnight in 5 mL of YPD and samples submitted for HPLC analysis (FIG. 5). The strain produced more lactic acid than the previously described R. oryzae LDH transformant, M16141 (see Example 1).

### Example V—Production of Vanillin in a Distilling Strain

Exogenous ferulic acid can be converted to vanillin by engineering the heterologous feruloyl-CoA (FCS) and feruloyl-CoA hydratase (ECH) genes (as shown in the biosynthetic pathway below).

Biosynthetic Pathway I for the Production of Vanillin from Ferulic Acid.

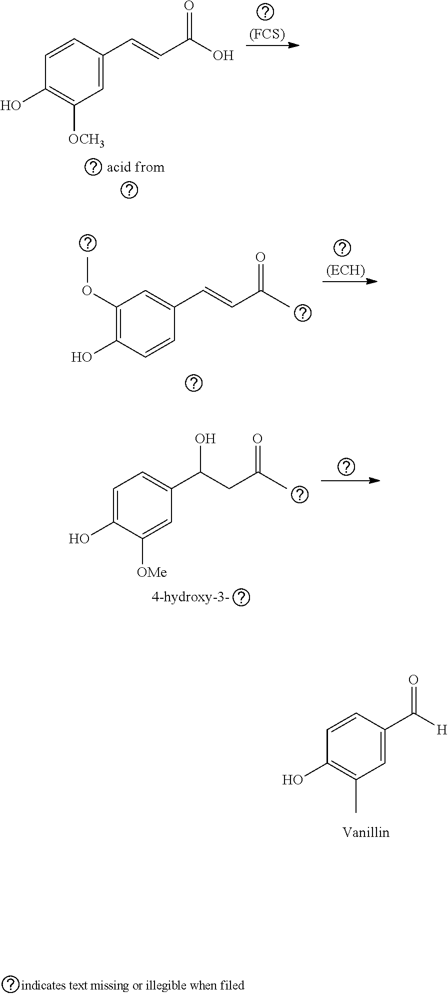

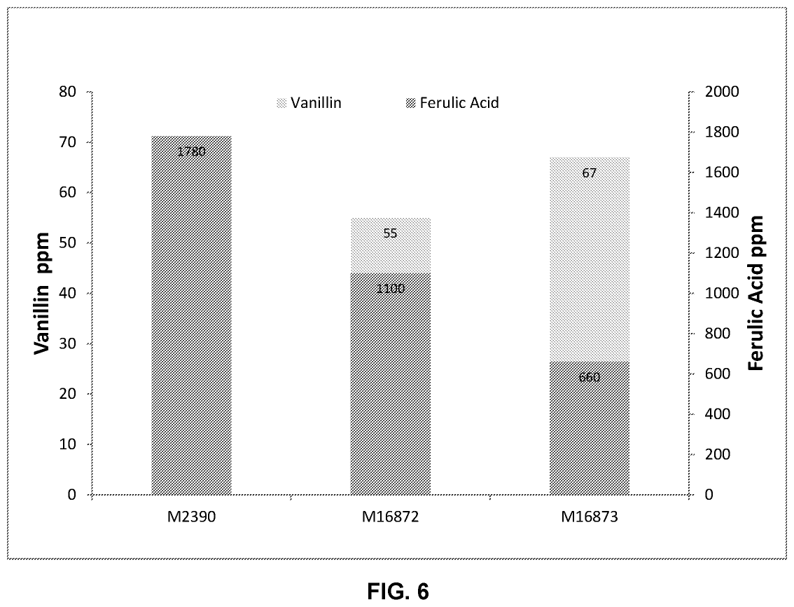

In this Example, it was achieved by expressing FCS and ECH into the distilling strain M2390 at the pad1 site, removing the pad1 open reading frame to eliminate the yeast's ability to decarboxylate ferulic acid into 4-vinyl guaiacol. The pad1 site was premarked using a Kan-MX negative selection cassette containing the TDK gene which results in sensitivity to the compound fluoro-deoxyuracil (FUDR), FCS and ECH genes were sourced from either Pseudomonas fluorescens (SEQ ID NO: 38 and SEQ ID NO: 43), as found in transformant M16872, or from Streptomyces sp, V-1 as found in transformant M16873 (SEQ ID NO: 39 and SEQ ID NO: 44), The FCS, for either the Pseudomonas or Streptomyces gene was under control of the constitutive TEF2 promoter and the ECH under control of the ADH1 promoter. Transformants were selected on YPD-FUDR plates to select for the removal of the Kan-MX cassette. Transformants were grown overnight in 5 mL of YP-dextrose 40 g/L with the addition of 2000 ppm ferulic acid, with vanillin production and residual ferulic acid measured via HPLC. As shown in FIG. 6, both pathways provided vanillin production at either 55 ppm or 67 ppm vanillin, compared to 0 ppm production from the parent strain.

### Example VI—Production of Vanillin in a Brewing Strain

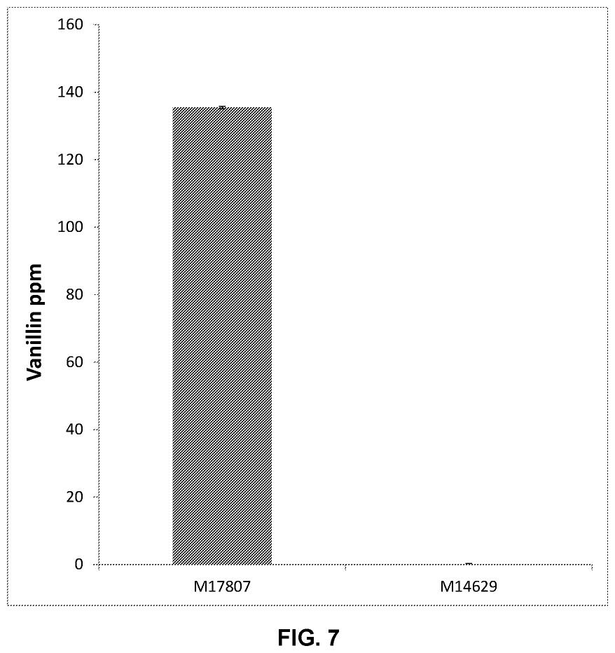

In this Example, vanillin expression was achieved by expressing FCS and ECH into the brewing strain M14629 at the pad1 site, removing the pad1 open reading frame to eliminate the yeast's ability to decarboxylate ferulic acid into 4-vinyl guaiacol. The pad1 site was premarked using a Kan-MX negative selection cassette containing the TDK gene which results in sensitivity to the compound fluoro-deoxyuracil (FUDR). FCS and ECH genes were sourced from Streptomyces sp. V-1 as found in transformant M17807 (SEQ ID NO: 39 and SEQ ID NO: 44). The FCS, for either the Pseudomonas or Streptomyces gene was under control of the constitutive TEF2 promoter and the ECH under control of the ADH1 promoter. A single transformant was grown overnight in 5 mL of YPD₄₀ and subsequently inoculated into a dry malt extract (DME) fermentation containing 13° Plato DME, 0.01% isomerized hop oil, 2000 ppm ferulic acid, at 175 mL volumes in 250 mL conical tubes. Samples were collected after 144 h of fermentation at room temperature and analyzed for vanillin production via HPLC. As shown in FIG. 7, the transformed brewing strain, M17807, produced 136 ppm vanillin compared to no vanillin production in the parent M14629

### Example VII—Production of Rasberry Ketone in a Brewing Strain

A de novo pathway for production of the raspberry ketone, [4-(4-hydroxyphenyl)butan-2-one], was engineered into an ale brewing strain, M14629. The pathway converts phenylalanine to cinnamic acid via the phenylalanine ammonium lyase (PAL) enzyme, followed by hydroxylation to p-coumaric acid via cinnamate-4-hydrolase (C4H). The p-coumaric is converted to coumaroyl-CoA by coumarate-CoA ligase and subsequently synthesized to benzylacetone using malonyl-CoA via benzylacetone synthase (BAS), and further reduced to the raspberry ketone via the yeast's native benzylacetone reductase activity (Synthetic pathway II).

Biosynthetic Pathway II for the Production of the [4-(4-hydroxyphenyl)butan-2-one](Raspberry Ketone).

All enzymes in the pathway can be heterologous, except for the final benzylacetone reductase which is a native activity found in S. cerevisiae.

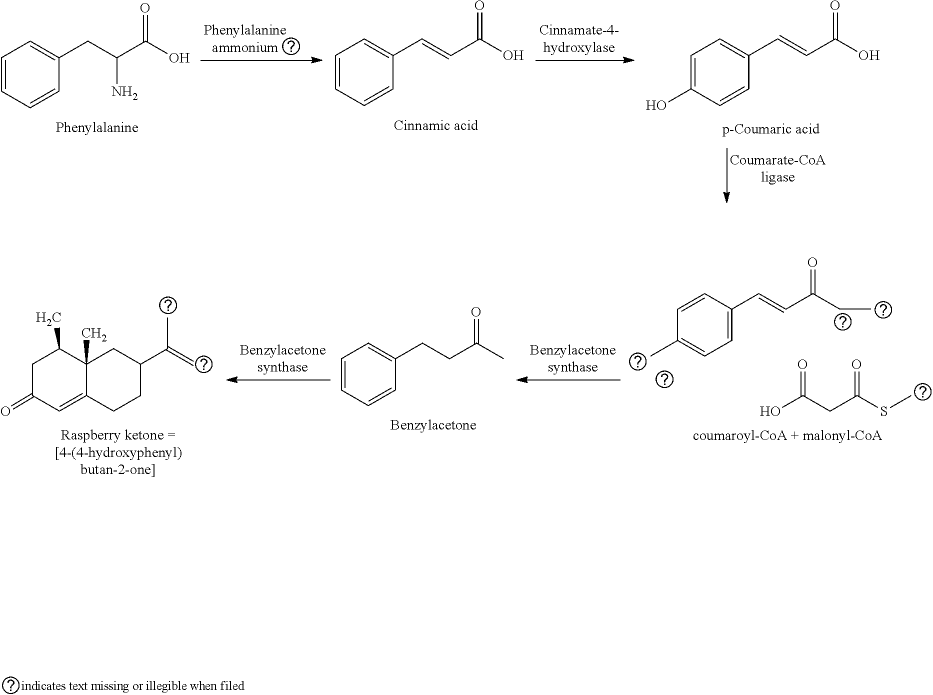

Because coumaric acid is an intermediate, the heterologous expression cassette was integrated at the pad1 site to prevent 4-vinyl phenol production. Both of the transformants M17735 and M17736 were engineered using the Rhodosporidium toruloides phenylalanine ammonium lyase (RtPAL; SEQ ID NO: 78) under control of the ADH1 promoter and the Arabidopsis thaliana cinnimate hydroxylase (AtC4H; SEQ ID NO: 80) under control of the TDH1 promoter. However, separate fusion proteins were used for the coumarate-CoA ligase and benzalacetone synthase. For M17735, the coumarate-CoA ligase was sourced from Arabidopsis thaliana (SEQ ID NO: 83) and the benzalacetone synthase from Rheum palmatum (SEQ ID NO: 60). In M17736, the coumarate-CoA ligase was sourced from Petroselinum crispum (SEQ ID NO: 84) along with the same R. palmatum benzalacetone synthase (SEQ ID NO: 60). The fusion coumarate-CoA ligase and benzalacetone synthase proteins were formed by removing the stop codon of the coumarate-CoA ligase gene sequences, introducing a linker with the amino acid sequence VDEAAAKSGR (SEQ ID NO: 85) and fusing to the R. palmatum benzalacetone synthase with the ATG start codon removed (SEQ ID NO: 81 and 82). Both expression cassettes were under control of the TEF2 promoter.

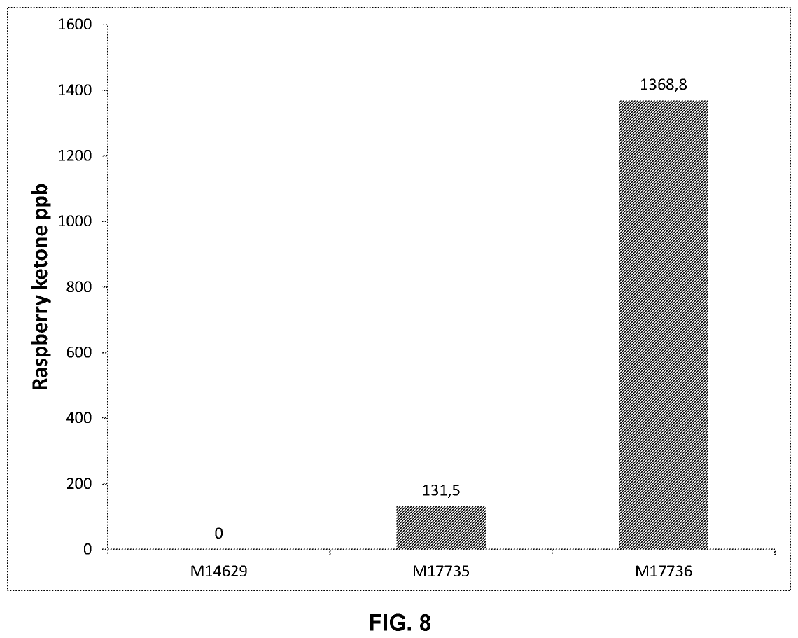

The transformants, M17735 and M17736, were grown overnight in 5 mL of YP-dextrose 40 g/L and subsequently inoculated into a dry malt extract (DME) fermentation containing 13′ Plato DME, 0.01% isomerized hop oil at 175 ml volumes in 250 mL conical tubes. Samples were collected after 144 h of fermentation at room temperature and analyzed for raspberry ketone production via GC/MS. As seen in FIG. 8, strain M17735 produced 131.5 parts per billion (ppb) of the raspberry ketone, whereas strain M17736 was able to produce 1369 ppb of the raspberry ketone.

### Example IX—Modulation of Lactic Acid Production During Wort Fermentation Using Co-Fermentations

Sour beers often have varying concentrations of lactic acid dependent on the organism used, method of souring, recipe, or style of beer. To demonstrate varying lactic production during a primary fermentation, the LDH expressing strain, M16141, was co-fermented with the parent strain, M14629 at various ratios. Using a 13*Plato dry malt extract wort containing 0.01% isomerized hop oil, the strains were pitched at a final 0.125 g/L dry cell weight concentration using ratios varying from 80:20 to 20:80 and compared to the separate 100% pitch for each strain. Fermentations were performed in duplicate with 13° Plato dry malt extract and 0.01% hop oil using 250 mL conical tubes at 175 mL volumes and incubated at 20° C. Co-cultures were split using a total 0.125 g/L dose ranging from 80% M16141 and 20% M14629 to 20% M16141 to 80% M14629 using lab-produced cream yeast. Lactic acid and ethanol production were measured using HPLC.

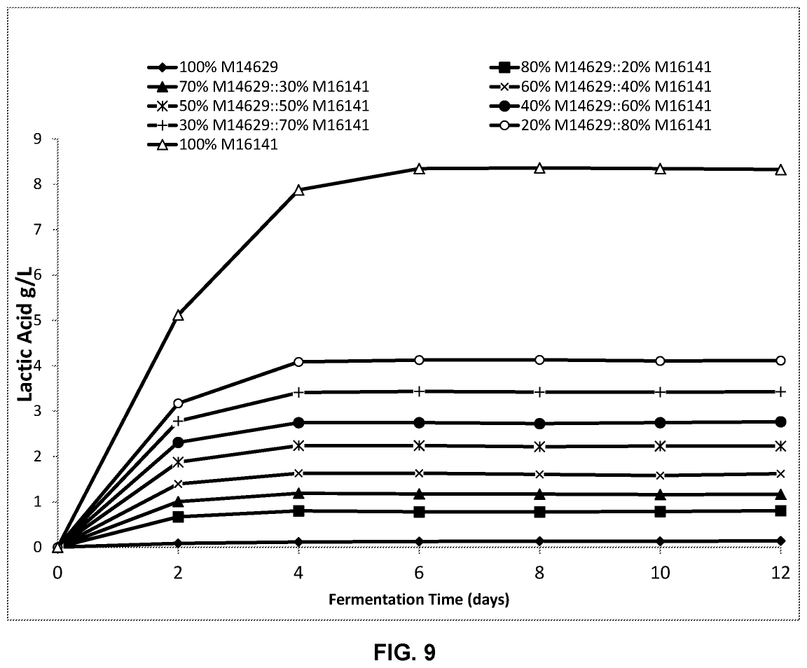

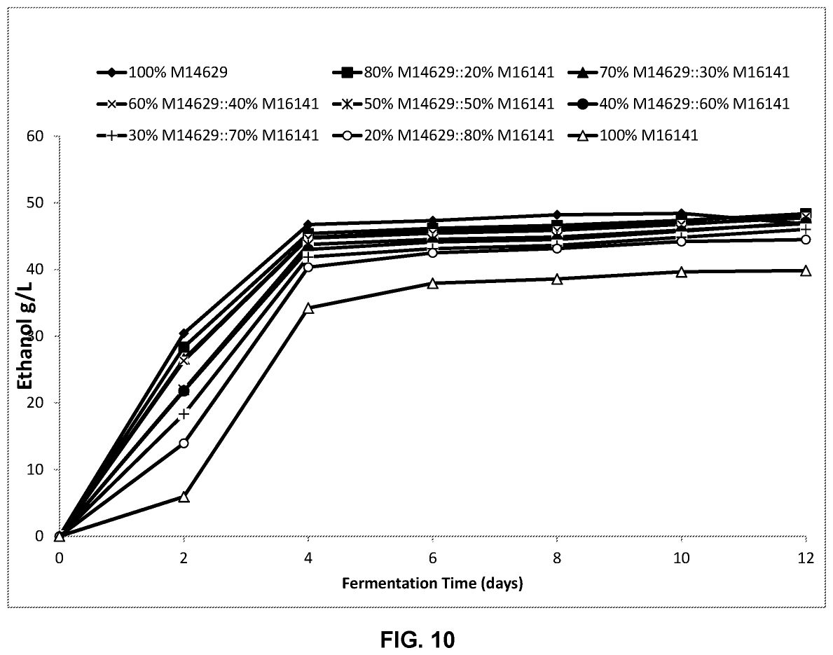

As seen in FIG. 9, the 100% M16141 pitch produced 8.3 g/L lactic acid after 6 days, however, under the conditions used, the lower ratios of M16141 did not produce theoretical lactic concentrations. For example, the 80% M16141 plus 20% M14629 combination produced half the amount of 100% M16141 at 4.1 g/L. Similarly, the 70:30, 60:40, 50:50, 40:60, 30:70, and 20:80 ratios of M16141:M14629 produced 3.4 g/L, 2.7 g/L, 2.2 g/L, 1.6 g/L, 1.2 g/L and 0.8 g/L lactic, respectively, as compared to a negligible 0.1 g/L production from the 100% M14629 fermentation. This is likely due to the slower growth of M16141, allowing the parent to slightly overtake the fermentation, as evidenced by the slower drop in specific gravity and ethanol production (data not shown). FIG. 10 depicts the ethanol kinetics during the same fermentation.

### Example X—Modulation of Lactic Acid Production Using Different Promoters

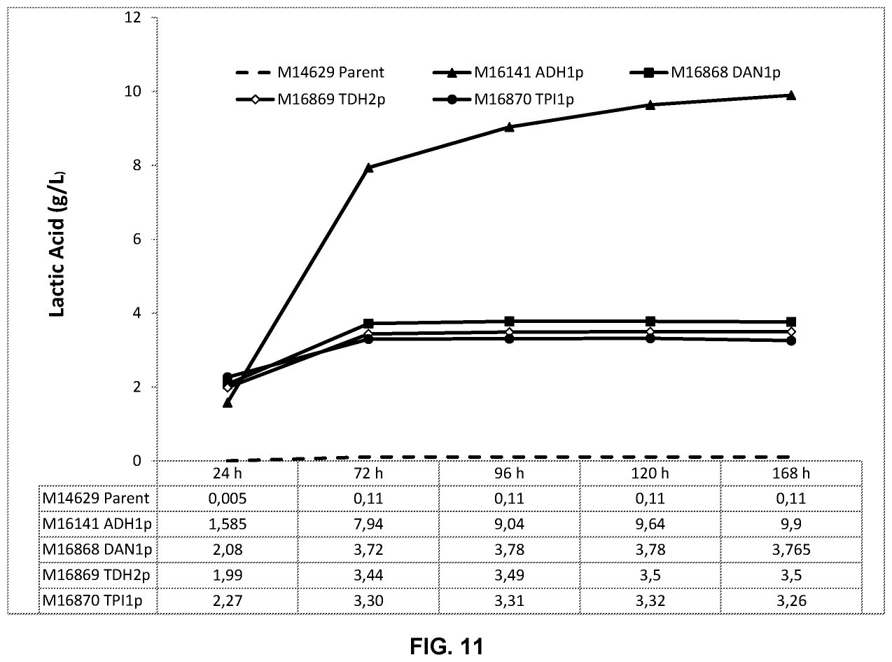

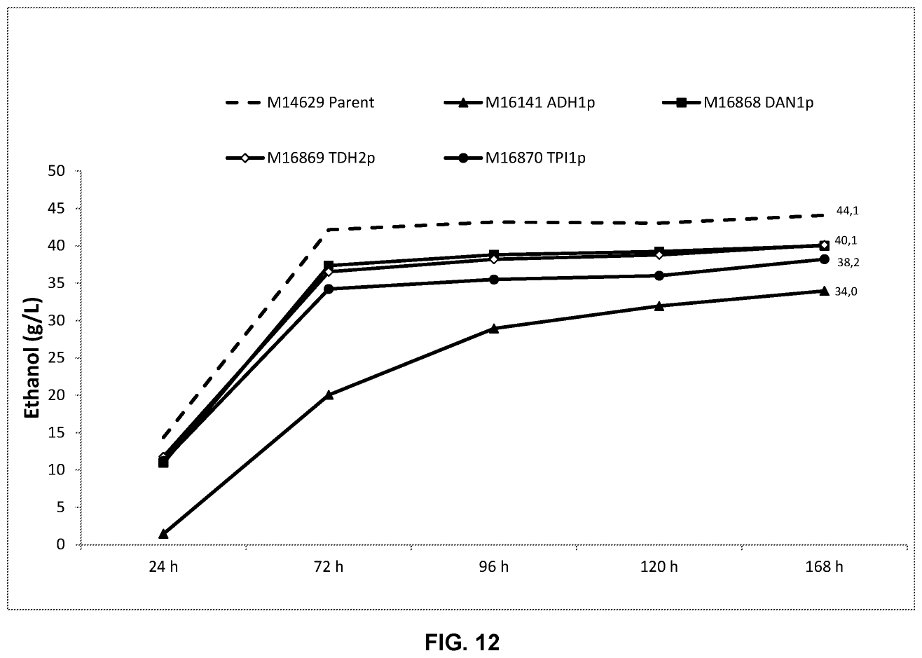

The lactate dehydrogenase containing cassette was engineered into an ale brewing strain, M14629, with Rhizopus oryzae lactate dehydrogenase (SEQ ID NO: 2) at the FCY1 site under control of various promoters. The previously described constitutive promoter, ADH1 promoter (adh1p) engineered into M16141 was compared to slightly weaker promoters from the native S. cerevisiae genes DAN1 (M16868), TDH2 (M16869), and TP11 (M16870). Successfully transformed cells grew on YPD₄₀ agar plates containing 5-fluorocytosine. The resulting transformants were screened for lactic production in a 13° Plato dry malt extract wort fermentation with 0.01% hop oil using 250 mL conical tubes at 175 mL volumes and incubated at 20° C. All 3 alternative promoters produced similar concentrations of lactic, nearly ⅓ of the M16141 strain (FIG. 11), with the strongest being the DAN1p producing a final 3.7 g/L, followed by TDH2p producing 3.5 g/L and TPI1 with 3.3 g/L. Similarly, the strains produced slightly less ethanol than the parent, M14629, but still more than M16141 (FIG. 12).

### Example XI—Heterologous Lactate Dehydrogenase Expression in a Lager Strain

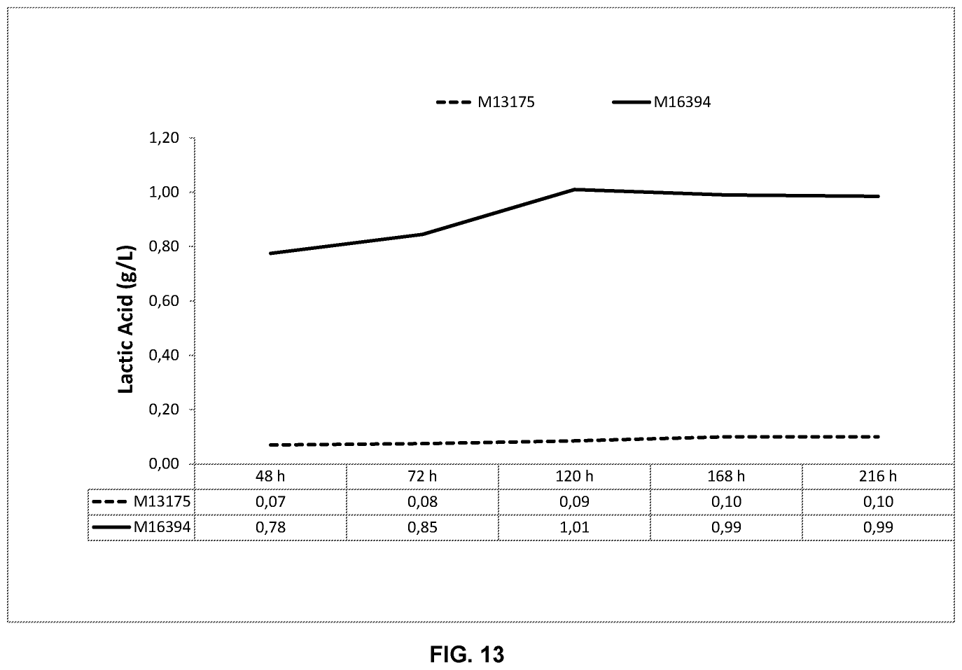

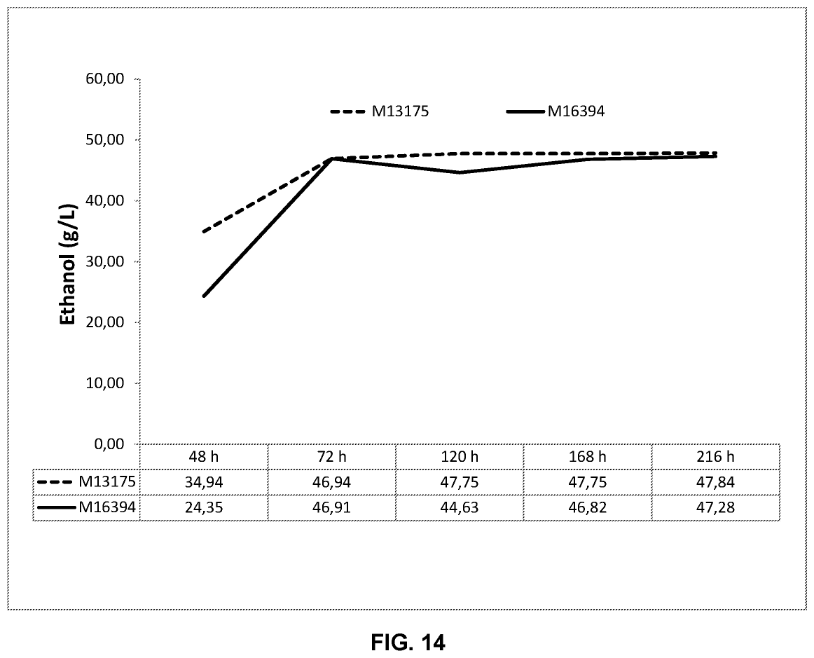

The lactate dehydrogenase containing cassette was engineered into a lager (Saccharomyces pastorianus) brewing strain, M13175, with Rhizopus oryzae lactate dehydrogenase (SEQ ID NO: 2) under control of the ADH1 promoter at the FCY1 site to generate strain M13175. Successfully transformed cells grew on YPD₄₀ agar plates containing 5-fluorocytosine. The resulting transformant, M16394, was screened for lactic production in a 12*Plato dry malt extract wort fermentation with 0.01% hop oil using 250 mL conical tubes at 175 mL volumes and incubated at 10° C. The LDH expressing lager strain, M16394, produced a maximum of 1 g/L lactic acid at lagering temperatures (FIG. 13) with slower, but similar final ethanol titers as the parent (FIG. 14).

The M16394 LDH transformant was further tested for lactic acid production in an ale fermentation at 20° C. and compared to the M16141 LDH expressing ale strain (described in Example I), along with the respective parents M13175 and M14629. Fermentations were performed in duplicate with 12*Plato dry malt extract and 0.01% hop oil using 250 mL conical tubes at 175 mL volumes and incubated at 20° C.

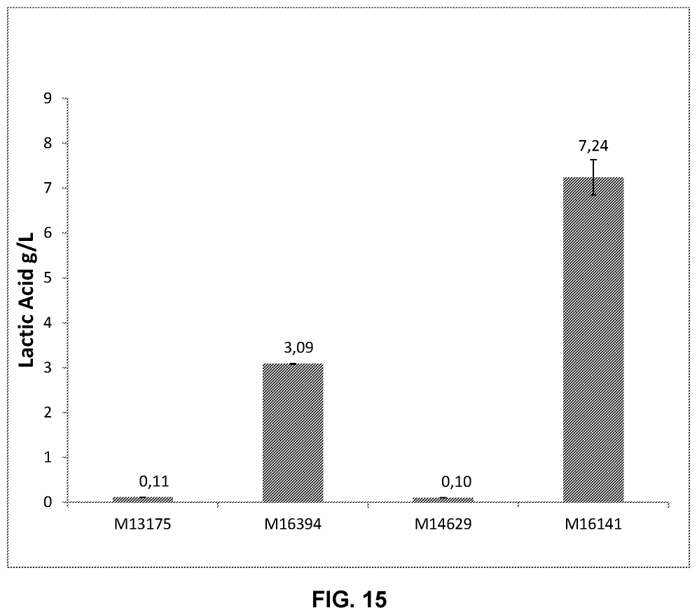

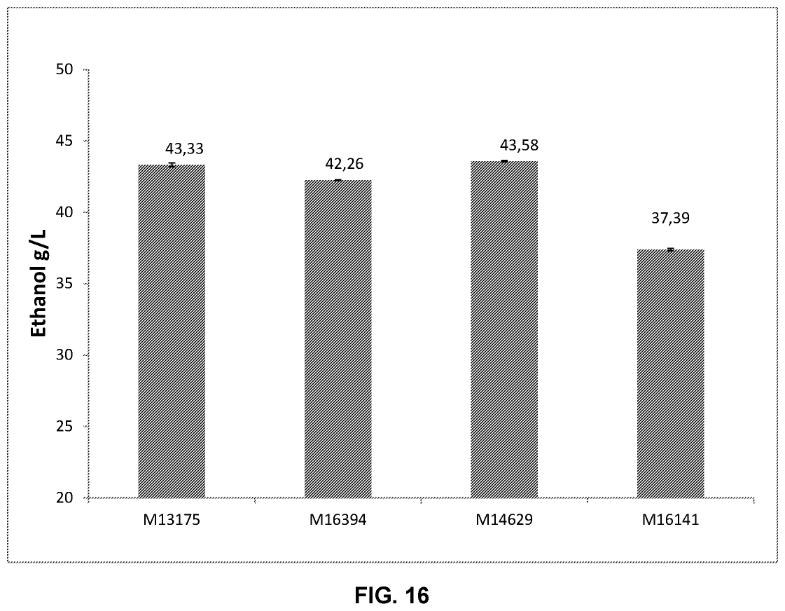

As shown in FIG. 15, strain M16394 produced 3.1 g/L lactic acid at ale temperatures compared to 7.2 g/L lactic production with strain M16141. As shown in FIG. 16, M16394 had only slightly lower ethanol production at 42.2 g/L compared to 43.3 and 43.5 g/L for the parent strains, while M16141 produced 37.4 g/L.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Adachi, E., Torigoe, M., Sugiyama, M., Nikawa, J.-I., & Shimizu, K. (1998). Modification of metabolic pathways of Saccharomyces cerevisiae by the expression of lactate dehydrogenase and deletion of pyruvate decarboxylase genes for the lactic acid fermentation at low pH value. Journal of Fermentation and Bioengineering, 86(3), 284-289.
Cankar K, van Houwelingen A, Goedbloed M, Renirie R, de Jong R M, Bouwmeester H, Bosch D, Sonke T, Beekwilder J. Valencene oxidase CYP706M1 from Alaska cedar (Callitropsis nootkatensis). FEBS Lett. 2014 Mar. 18; 588(6):1001-7. doi: 10.1016/j.febslet.2014.01.061. Epub 2014 Feb. 11.
De Keersmaecker, J., 1996. The Mystery of Lambic Beer. Sci. Am. 275, 74-80.
Goward C R, Nicholls D J. Malate dehydrogenase: a model for structure, evolution, and catalysis. Protein Sci. 1994 October; 3(10):1883-8.
Osburn, K., Amaral, J., Metcalf, S. R., Nickens, D. M., Rogers, C. M., Sausen, C., Bochman, M. L. (2018). Primary souring: A novel bacteria-free method for sour beer production. Food Microbiology, 70 (Supplement C), 76-84. https://doi.org/10.1016/j.fm.2017.09.007
Porro, D., Brambilla, L., Ranzi, B. M., Martegani, E., Alberghina, L., 1995. Development of Metabolically Engineered Saccharomyces cerevisiae Cells for the Production of Lactic Acid. Biotechnol. Prog. 11. 294-298. https//doi.org/10.1021/bp00033a009
Sauer, M., Porro, D., Mattanovich, D., & Branduardi, P. (2010). 16 years research on lactic acid production with yeast—ready for the market? Biotechnology and Genetic Engineering Reviews, 27(1), 229-256. https://doi.org/10.1080/02648725.2010.10648152
Skory, C. D. (2000). Isolation and Expression of Lactate Dehydrogenase Genes from Rhizopus oryzae. Applied and Environmental Microbiology, 66(6), 2343-2348.
Skory, C. D. (2003). Lactic acid production by Saccharomyces cerevisiae expressing a Rhizopus oryzae lactate dehydrogenase gene. Journal of Industrial Microbiology and Biotechnology, 30(1), 22-27. https://doi.org/10.1007/s10295-002-0004-2
Spitaels, F., Wleme, A. D., Janssens, M., Aerts, M., Daniel, H.-M., Landschoot, A. V., Vuyst, L. D., Vandamme, P., 2014. The Microbial Diversity of Traditional Spontaneously Fermented Lambic Beer. PLOS ONE 9, e95384.
Wriessnegger T, Augustin P, Engleder M, Leitner E, Müller M, Kaluzna I, Schürmann M, Mink D, Zellnig G, Schwab H, Pichler H. Production of the sesquiterpenoid (+)-nootkatone by metabolic engineering of Pichia pastoris. Metab Eng. 2014 July; 24:18-29.
Wright S K, Viola R E. Alteration of the specificity of malate dehydrogenase by chemical modulation of an active site arginine. J Biol Chem. 2001 Aug. 17; 276(33):31151-5.

1. A recombinant yeast host cell for making a flavoured alcoholic beverage obtained by fermentation of a fermentation medium, the recombinant yeast host cell:
has an heterologous nucleic acid molecule encoding one or more heterologous polypeptide for the production of a flavour compound, wherein the heterologous nucleic acid molecule allows the production the flavour compound in the fermentation medium;
has a native ethanol production pathway; and
accumulates at least 5 g/L of ethanol during the fermentation.

2. The recombinant yeast host cell of claim 1, wherein the flavour compound comprises lactic acid.

3. The recombinant yeast host cell of claim 2, wherein the one or more heterologous polypeptide comprises an enzyme having lactacte dehydrogenase (LDH) activity.

4.-11. (canceled)

12. The recombinant yeast host cell of claim 1, wherein the flavour compound comprises valencene.

13. The recombinant yeast host cell of claim 12, wherein the one or more heterologous polypeptide comprises:
a heterologous farnesyl diphosphate synthase (FDPS) enzyme, a variant thereof or a fragment thereof; and/or
a heterologous valencene synthase enzyme, a variant thereof or a fragment thereof.

14.-16. (canceled)

17. The recombinant yeast host cell of claim 1, wherein the flavour compound comprises nootkatone.

18. The recombinant yeast host cell of claim 17, wherein the one or more heterologous polypeptide comprises:
a heterologous farnesyl diphosphate synthase (FDPS) enzyme, a variant thereof or a fragment thereof;
a heterologous valencene synthase enzyme, a variant thereof or a fragment thereof;
a heterologous cytochrome P450 oxygenase enzyme, a variant thereof or a fragment thereof;
a heterologous cytochrome hydroxylase enzyme, a variant thereof or a fragment thereof;
a heterologous cytochrome P450 reductase enzyme; and/or
a heterologous valencene oxidase enzyme.

19.-29. (canceled)

30. The recombinant yeast host cell of claim 1, wherein the flavour compound comprises vanillin.

31. The recombinant yeast host cell of claim 30, wherein the one or more heterologous polypeptide comprises;
a heterologous feruloyl-CoA synthase (FCS) enzyme, a variant thereof or a fragment thereof;
a heterologougs enoyl-CoA hydratase (ECH) enzyme, a variant thereof or a fragment thereof; and/or
a heterologous vanillin synthase enzyme.

32.-38. (canceled)

39. The recombinant yeast host cell of claim 30, lacking phenylacrylic acid decarboxylase enzymatic activity.

40. The recombinant yeast host cell of claim 1, wherein the flavour compound comprises isoamyl acetate.

41. The recombinant yeast host cell of claim 40, wherein the one or more heterologous polypeptide comprises an heterologous alcohol acetyl transferase (ATF) enzyme, a variant thereof or a fragment thereof.

42.-45. (canceled)

46. The recombinant yeast host cell of claim 40 overexpressing a native alcohol acetyl transferase (ATF) enzyme.

47. The recombinant yeast host cell of claim 1, wherein the flavour compound comprises 4-(4-hydroxyphenyl)-2-butanone.

48. The recombinant yeast host cell of claim 47, wherein the one or more heterologous polypeptide comprises;
a heterologous phenylalanine-ammonium lyase (PAL) enzyme, a variant thereof or a fragment thereof;
a heterologous cinnimate-4-hydroxylase (C4H) enzyme, a variant thereof or a fragment thereof;
a heterologous coumarate-CoA ligase (4CL) enzyme, a variant thereof or a fragment thereof;
a heterologous benzalacetone synthase (BAS) enzyme, a variant thereof or a fragment thereof; and/or
a chimeric enzyme comprising an heterologous coumarate-CoA ligase (4CL) enzyme moiety and an heterologous benzalacetone synthase (BAS) enzyme moiety.

49.-61. (canceled)

62. The recombinant yeast host cell of claim 47 overexpressing a native benzalactone reductase.

63. The recombinant yeast host cell of claim 1, wherein the flavour compound comprises 4-ethyl-phenol and/or 4-ethyl guiacol.

64. The recombinant yeast host cell of claim 63, wherein the one or more heterologous polypeptide comprises a heterologous vinylphenol reductase (VPR) enzyme, a variant thereof or a fragment thereof.

65. (canceled)

66. The recombinant yeast host cell of claim 1, wherein the flavour compound comprises phenylethyl alcohol.

67. The recombinant yeast host cell of claim 66, wherein the one or more heterologous polypeptide comprises:
a heterologous ARO8 enzyme, a variant thereof or a fragment thereof;
a heterologous ARO9 enzyme, a variant thereof or a fragment thereof;
a heterologous PDC1 enzyme, a variant thereof or a fragment thereof;
a heterologous PDC5 enzyme, a variant thereof or a fragment thereof;
a heterologous PDC6 enzyme, a variant thereof or a fragment thereof;
a heterologous ARO10 enzyme, a variant thereof or a fragment thereof;
a heterologous SFA1 enzyme, a variant thereof or a fragment thereof;
a heterologous ADH4 enzyme, a variant thereof or a fragment thereof; and/or
a heterologous ADH5 enzyme, a variant thereof or a fragment thereof.

68.-75. (canceled)

76. The recombinant yeast host cell of claim 1, wherein the flavour compound comprises ethyl capraote.

77. The recombinant yeast host cell of claim 76, wherein the one or more heterologous polypeptide comprises a heterologous mutated FAS2 enzyme, a variant thereof or a fragment thereof.

78. The recombinant yeast host cell of claim 1, wherein the flavour compound comprises vanillyloctanamide.

79. The recombinant yeast host cell of claim 78, wherein the one or more heterologous polypeptide comprises:
a heterologous capsaicin synthase enzyme, a variant thereof or a fragment thereof; and/or
a heterologous pAMT1 enzyme, a variant thereof or a fragment thereof.

80.-90. (canceled)

91. The recombinant yeast host cell of claim 1 being from genus Saccharomyces sp., from species Saccharomyces cerevisiae or from species Saccharomyces pastorianus.

92.-94. (canceled)

95. A fermenting agent for making a flavoured and fermented alcoholic beverage comprising the recombinant yeast host cell of claim 1 and a nutrient.

96. (canceled)

97. A combination for making a flavoured and fermented alcoholic beverage comprising or consisting essentially of the recombinant yeast host cell of claim 1 and a non-genetically modified yeast.

98. (canceled)

99. A process for making a flavoured and fermented alcoholic beverage, the process comprising (i) contacting the recombinant yeast host cell of claim 1 with a substrate comprising carbohydrates to provide a mixture; and (ii) fermenting the mixture to accumulate the flavor compound and at least 5 g/L of ethanol in the fermented mixture.

100.-103. (canceled)

104. A process for making a beer, the process comprising (i) contacting the recombinant yeast host cell of claim 1 with a substrate comprising carbohydrates to provide a mixture; and (ii) fermenting the mixture so as to accumulate the flavor compound and at least 5 g/L of ethanol in the fermented mixture.

105.-109. (canceled)
